SEQUENCE LISTING (30 sequences)

SEQ ID NO: 1 - DNA, 29 bp, Artificial Sequence
  YS1646 SopE2 promoter and secretory signal forward primer
ccgctcgagtaaaaatgttcctcgataaa

SEQ ID NO: 2 - DNA, 20 bp, Artificial Sequence
  YS1646 SopE2 promoter and secretory signal reverse primer
catggtagttctccttttag

SEQ ID NO: 3 - DNA, 27 bp, Artificial Sequence
  YS1646 SptP promoter and secretory signal forward primer
cgcctcgagtttacgctgactcattgg

SEQ ID NO: 4 - DNA, 23 bp, Artificial Sequence
  YS1646 SptP promoter and secretory signal reverse primer
catttttctctcctcatacttta

SEQ ID NO: 5 - DNA, 27 bp, Artificial Sequence
  YS1646 SseJ promoter and secretory signal forward primer
cgcctcgagacataaaacactagcact

SEQ ID NO: 6 - DNA, 27 bp, Artificial Sequence
  YS1646 SseJ promoter and secretory signal reverse primer
cgcctcgagacataaaacactagcact

SEQ ID NO: 7 - DNA, 26 bp, Artificial Sequence
  YS1646 SspH1 promoter and secretory signal forward primer
cgcctcgagcgctatatcaccaaaac

SEQ ID NO: 8 - DNA, 29 bp, Artificial Sequence
  YS1646 SspH1 promoter and secretory signal reverse primer
ctctgcggccgcggtaagacctgacgctc

SEQ ID NO: 9 - DNA, 24 bp, Artificial Sequence
  YS1646 SspH2 promoter and secretory signal forward primer
cgcctcgaggtttgtgcgtcgtat

SEQ ID NO: 10 - DNA, 29 bp, Artificial Sequence
  YS1646 SspH2 promoter and secretory signal reverse primer
ctctgcggccgcattcaggcaggcacgca

SEQ ID NO: 11 - DNA, 25 bp, Artificial Sequence
  YS1646 SteA promoter and secretory signal forward primer
cgcctcgaggtttcgccgcatgttg

SEQ ID NO: 12 - DNA, 29 bp, Artificial Sequence
  YS1646 SteA promoter and secretory signal reverse primer
ctctgcggccgcataattgtccaaatagt

SEQ ID NO: 13 - DNA, 25 bp, Artificial Sequence
  YS1646 SteB promoter and secretory signal forward primer
cgcctcgagcgctccagcgcttcga

SEQ ID NO: 14 - DNA, 28 bp, Artificial Sequence
  YS1646 SteB promoter and secretory signal reverse primer
ctctgcggccgctctgacattaccattt

SEQ ID NO: 15 - DNA, 78 bp, Artificial Sequence
  Lac promoter
cgcctcgagcattaggcaccccaggctttacactttatgcttccggctcgtatgttgtgtggaattgtgagcggataa

SEQ ID NO: 16 - DNA, 71 bp, Artificial Sequence
  Lac promoter
gtggaattgtgagcggataacaatttcacacaggaaacagctatgaccatgactaacataacactatccac

SEQ ID NO: 17 - DNA, 28 bp, Artificial Sequence
  DH5D E. coli nirB promoter forward primer
cgcctcgagttgtggttaccggcccgat

SEQ ID NO: 18 - DNA, 31 bp, Artificial Sequence
  DH5D E. coli nirB promoter reverse primer
cgcgcggccgccggatctttactcgcattac

SEQ ID NO: 19 - DNA, 27 bp, Artificial Sequence
  YS1646 pagC promoter forward primer
cgcctcgaggttaaccactcttaataa

SEQ ID NO: 20 - DNA, 20 bp, Artificial Sequence
  YS1646 pagC promoter reverse primer
aacaactccttaatactact

SEQ ID NO: 21 - DNA, 53 bp, Artificial Sequence
  YS1646 SopE2 Secretion Signal forward primer
ggcggtaatagaaaagaaatcgaggcaaaaatgactaacataacactatccac

SEQ ID NO: 22 - DNA, 28 bp, Artificial Sequence
  YS1646 SopE2 Secretion Signal reverse primer
aagtcgcggccgccggatctttactcgc

SEQ ID NO: 23 - DNA, 58 bp, Artificial Sequence
  YS1646 SspH1 Secretion Signal forward primer
ggcggtaatagaaaagaaatcgaggcaaaaatgtttaatatccgcaatacacaacctt

SEQ ID NO: 24 - DNA, 29 bp, Artificial Sequence
  YS1646 SspH1 Secretion Signal reverse primer
ctctgcggccgcggtaagacctgacgctc

SEQ ID NO: 25 - DNA, 27 bp, Artificial Sequence
  VPI 10463 rTcdA forward primer
cgcgcggccgcgacttattactatgat

SEQ ID NO: 26 - DNA, 29 bp, Artificial Sequence
  VPI 10463 rTcdA reverse primer
tagtcggcgcgcccgccatatatcccagg

SEQ ID NO: 27 - DNA, 30 bp, Artificial Sequence
  VPI 10463 rTcdB forward primer
ccggcggccgcagagaaattttatattaat

SEQ ID NO: 28 - DNA, 31 bp, Artificial Sequence
  VPI 10463 rTcdB reverse primer
agtcggcgcgccgttcactaatcactaattg

SEQ ID NO: 29 - DNA, 27 bp, Artificial Sequence
  pEGFP_C1 EGFP forward primer
cgcgcggccgcggtgagcaagggcgag

SEQ ID NO: 30 - DNA, 30 bp, Artificial Sequence
  pEGFP_C1 EGFP reverse primer
agtcggcgcgccttacttgtacagctcgtc

(19) United States
(12) Patent Application Publication
(10) Pub. No.: US 2020/0093912 A1
(43) Pub. Date: Mar. 26, 2020

(54) VACCINE AND METHOD OF VACCINATION AGAINST CLOSTRIDIUM DIFFICILE USING A LIVE BACTERIAL VECTOR

(71) Applicant: THE ROYAL INSTITUTION FOR THE ADVANCEMENT OF LEARNING/MCGILL UNIVERSITY, Montreal (CA)

(72) Inventors: Brian J. Ward, Montreal (CA); Kaitlin Grace Frances Winter, Montreal (CA)

(73) Assignee: THE ROYAL INSTITUTION FOR THE ADVANCEMENT OF LEARNING/MCGILL UNIVERSITY, Montewal (CA)

(21) Appl. No.: 16/576,527

(22) Filed: Sep. 19, 2019

Related U.S. Application Data
(60) Provisional application No. 62/803,167, filed on Feb. 8, 2019.
(60) Provisional application No. 62/734,103, filed on Sep. 20, 2018.

(51) Int. Cl. A61K 39/112; A61K 9/00

(52) U.S. Cl. CPC A61K 39/0275; A61K 9/0053

(57) ABSTRACT

An attenuated Salmonella enterica serovar Typhimurium strain (YS1646) is repurposed to produce a vaccine. Plasmid-based candidates expressing either the TcdA or TcdB RBD were screened. Different vaccine routes and schedules were tested to achieve detectable serum and mucosal antibody titers in C57BL/6J mice. When given in a multi-modality schedule over 1 week (day 0 IM+PO, days 2 and 4 PO), several candidates provided 100% protection against lethal challenge. Substantial protection (82%) was achieved with combined PO TcdA/TcdB vaccination alone (d0, 2 and 4). These data demonstrate the potential of the YS1646-based vaccines for C. difficile.

### CROSS REFERENCE TO RELATED APPLICATIONS

The present application is a non-provisional of, and claims benefit of priority under 35 U.S.C. § 119(e) from, U.S. Provisional Patent Application No. 62/734,103, filed Sep. 20, 2018, and from U.S. Provisional Patent Application No. 62/803,167, filed Feb. 8, 2019, the entirety of which are expressly incorporated herein by reference for all purposes.

### BACKGROUND

### Field of the Invention

This invention is generally in the field of live bacterial vector vaccines and methods of administration thereof. In particular, the invention involves a live Salmonella vector genetically engineered to have Clostridium difficile antigens, e.g., toxins A and B (TcdA and TcdB), and methods of administration thereof.

### Description of the Prior Art

Citation or identification of any reference herein, in any section of this application, shall not be construed as an admission that such reference is available as prior art to the present application. The disclosures of each reference disclosed herein, whether U.S. or foreign patent literature, or non-patent literature, are hereby incorporated by reference in their entirety in this application, and shall be treated as if the entirety thereof forms a part of this application.

Such references are provided for their disclosure of technologies to enable practice of the present invention, to provide basis for claim language, to make clear applicant's possession of the invention with respect to the various aggregates, combinations, and subcombinations of the respective disclosures or portions thereof (within a particular reference or across multiple references). The citation of references is intended to be part of the disclosure of the invention, and not merely supplementary background information. The incorporation by reference does not extend to teachings which are inconsistent with the invention as expressly described herein, and is evidence of a proper interpretation by persons of ordinary skill in the art of the terms, phrase and concepts discussed herein, without being limiting as the sole interpretation available.

Genetically-engineered bacterial vectors represent a promising method of therapy for various diseases and as a biomolecule delivery system.

YS1646 is a highly attenuated Salmonella enterica serovar Typhimurium carrying mutations in the msbB (lipopolysaccharide or LPS) and purI (purine biosynthesis pathway) genes that was originally developed as a possible cancer therapeutic (Toso et al., 2002). Although its development was halted when it failed to provide benefit in a large phase I trial in subjects with advanced cancer, it was well-tolerated when administered intravenously at doses of up to 3.0×10⁸ colony-forming units/m² (Toso et al., 2002). YS1646 is repurposed as a novel vaccination platform and reasoned that a locally-invasive but highly attenuated Salmonella vector might induce both local and systemic responses to CatB. The flagellin protein of S. Typhimurium has been proposed as a general mucosal adjuvant through its action on toll-like receptor (TLR) 5 (Makvandi et al. 2018). Other Salmonella products such as LPS would be expected to further enhance immune responses by triggering TLR4 (Hayashi et al. 2001; Poltorak et al., 1998). Indeed, live attenuated Salmonella have multiple potential advantages as vaccine vectors and have been used to express foreign antigens against infectious diseases and cancers (Clark-Curtiss et al. 2018, Galen et al. 2016; Panthel et al. 2008). They directly target the intestinal microfold (M) cells overlying the gut-associated lymphoid tissues (GALT) (Galen et al. 2016; Jepson et al. 2001; Hohmann et al. 1995; Penha Filho et al. 2012; Sztein 2007), have large ‘carrying’ capacity (Miller et al. 1990) and are easy to manipulate both in the laboratory and at industrial scale. Although there is considerable experience with the attenuated S. typhi vaccine strain (Ty21a: Vivotif™) in the delivery of heterologous antigens (Galen et al. 2016; Gentschev et al. 2007), far less is known about the potential of other Salmonella strains. Of direct relevance to the current work, Chen and colleagues used YS1646 to express a chimeric S. japonicum antigen that induced both strong antibody and cellular responses after repeated oral dosing and provided up to 62% protection in a murine challenge model (Chen et al. 2011).

See, U.S. 20190017057; 20180271787; 20170157239; 20170051260; 20160222393; 20160028148; 20150017204; 20140220661; 20120142080; 20110223241; 20100136048; 20100135961; 20090169517; 20080124355; 20070009489; 20050255088; 20050249706; 20050052892; 20050036987; 20040219169; 20040042274; 20040037117; 20030170276; 20030113293; 20030109026; 20020026655; U.S. Pat. Nos. 10,364,435; 10,286,051; 10,188,722; 10,141,626; 10,087,451; 9,878,023; 9,739,773; 9,737,592; 9,657,085; 9,616,114; 9,597,379; 9,593,339; 9,486,513; 9,421,252; 9,365,625; 9,315,817; 9,200,289; 9,200,251; 9,068,187; 8,956,859; 8,771,669; 8,647,642; 8,623,350; 8,524,220; 8,440,207; 8,241,623; 7,514,089; 7,452,531; 7,354,592; 7,211,843; 6,962,696; 6,934,176; 6,923,972; 6,863,894; 6,798,684; 6,685,935; 6,475,482; 6,447,784; 6,190,657; and 6,080,849.

In recent years, live attenuated Salmonella has been increasingly used to express foreign antigens against infectious diseases and cancers (Clark-Curtiss et al. 2018; Galen et al. 2016; Panthel et al. 2008). Salmonella enterica is a facultative intracellular pathogen that replicates in a unique membrane-bound host cell compartment, the Salmonella-containing vacuole (Ibarra et al. 2009). Although this location limits exposure of both Salmonella and foreign proteins produced by the bacterium to the immune system, the organism's type III secretion systems (T3SS) can be exploited to translocate heterologous antigens into the host cell cytoplasm. Salmonella enterica encodes two distinct T3SS within the Salmonella pathogenicity islands 1 and 2 (SPI-I and SPI-II) that become active at different phases of infection (Gerlach et al. 2007).

The highly attenuated Salmonella enterica Typhimurium strain YS1646 had been used in a phase 1 clinical cancer trial at doses up to 3×10⁸ IV, and was shown to have a promising toxicity profile. Like all Salmonella enterica species, YS1646 has two distinct T3SS located in Salmonella pathogenicity islands 1 and 2 (SPI-I and SPI-II) (Haraga et al. 2008) that are active at different phases of infection (Gerlach et al. 2007). The SPI-I T3SS translocates proteins upon first contact of the bacterium with epithelium cells through to the stage of early cell invasion while SPI-II expression is induced once the bacterium has been phagocytosed (Le et al. 2000). These T3SS have been used by many groups to deliver heterologous antigens in Salmonella-based vaccine development programs (Panthel et al. 2008; Xiong et al. 2010; Galen et al. 2016).

In recent years, live attenuated Salmonella has been increasingly used to express foreign antigens against infectious diseases and cancers. (Clark-Curtiss et al. 2018; Galen et al. 2016; Panthel et al. 2008; Bolhassani et al. 2012; Medina et al. 2001; Seegers 2002; Shams et al. 2005; Kang et al. 2002; Cardenas et al. 1992; Buckley et al. 2010; Dougan et al. 1987; Mastroeni et al. 1992; Galen et al. 1997; Shahabi et al. 2010; Fraillery et al. 2007; Paterson et al. 2010; Wieckowski et al. 2017; Wieckowski et al. 2017; Wieckowski et al. 2018; Vendrell et al. 2016).

There is considerable experience in using the attenuated S. typhi vaccine strain (Ty21a: Vivotif™) in the delivery of heterologous antigens (Panthel et al. 2008). However, S. typhimurium YS1646 was selected for various reasons as a vector. This strain is attenuated by mutations in its msbB (LPS) and purI (purine biosynthesis pathway) genes and was originally developed as a non-specific ‘cancer vaccine’ for solid tumors. YS1646 was carried through pre-clinical and toxicity testing by Vion, Inc. in rodents, dogs and non-human primates before a phase I clinical trial where it ultimately failed (Clairmont et al. 2000; Toso et al. 2002). More recently, YS1646 has been used to express a chimeric Schistosoma japonicum antigen that was tested in a murine model of schistosomiasis (Chen et al. 2011). Repeated oral administration of one of the engineered strains elicited a strong systemic IgG antibody response, induced antigen-specific T cells and provided up to 75% protection against S. japonicum challenge.

The T3SS secretion system is discussed in U.S. 2019/0055569, 2010/0120124, 2012/0021517, 2015/0359909, U.S. Pat. Nos. 9,951,340, 6,306,387.

Some bacterial pathogens comprise a type three secretion system (T3SS), which serves as a needle-like system for delivering bacterial polypeptides (effectors) into host cells. These effector polypeptides typically contribute to the virulence of the bacterial cell. In contrast, commensal microbes have not been described to comprise a T3SS.

A T3SS is a multi-protein structure found in gram negative bacteria. It moves polypeptides from the cytoplasm of the bacterial cell through the interior of the T3SS “needle” into the cytoplasm of a target cell. T3SS's are found in pathogenic strains and have been observed in pathogenic isolates of, e.g., Shigella, Salmonella, E. coli, Burkholderia, Yersinia, Chlamydia, Pseudomonas, Erwinia, Ralstonia, Rhizobium, Vibrio, and Xanthamonas. Further discussion of T3SS's can be found (Izore et al. 2012; Wooldridge 2009; Snyder et al. 2007).

The suite of T3SS-related proteins in a given wild-type cell is typically divided into structural proteins (those proteins which form the needle itself), substrate proteins (those proteins which are transported through the needle to the host), and chaperones (those proteins that bind effectors in the cytoplasm to protect, process, and/or shuttle the effectors to the needle). As used herein, a “functional T3SS” refers, minimally, to the set of structural proteins which are required in order to transfer at least one polypeptide to a target cell. In some embodiments, a functional T3SS system can comprise one or more chaperone proteins. In some embodiments, a functional T3SS can comprise one or more, for example, two, three, or four, substrates which are not virulence factor (e.g. certain translocators). In some embodiments, a functional T3SS does not comprise a virulence factor which is delivered to the target cell.

As used herein, a “virulence factor” refers to those substrates which affect and/or manipulate a target cell in a manner which is beneficial to infection and deleterious to the target cell, i.e., they perturb the normal function of the target cell. Examples of actions of virulence factors include, but are not limited to, modulation of actin polymerization, induction of apoptosis, modulation of the cell cycle, modulation of gene transcription. Not all substrates are necessarily virulence factors. By way of non-limiting example, a T3SS (and a functional T3SS) can comprise proteins referred to as translocators. These substrates are secreted by the T3SS as it nears a complete form and create a pore in the target cell membrane, allowing further substrates to be delivered into the cytoplasm of the target cell, i.e., translocators are substrates in that they travel through the needle to the target cell and are also structural proteins in that they form part of the structure through which other substrates are delivered into the target cell. In some embodiments, a single polypeptide can be both a translocator and a virulence factor (e.g. IpaB of Shigella). A functional T3SS system can be introduced into a non-pathogenic bacterial cell.

Homologs of any given polypeptide or nucleic acid sequence can be found using, e.g., BLAST programs (freely available on the world wide web at blast.ncbi.nlm.nih.gov/), e.g. by searching freely available databases of sequence for homologous sequences, or by querying those databases for annotations indicating a homolog (e.g. search strings that comprise a gene name or describe the activity of a gene). The homologous amino acid or DNA sequence can be at least 90%, at least 95%, at least 96%, at least 97%, at least 98%, at least 99%, or more, identical to a reference sequence. The degree of homology (percent identity) between a reference and a second sequence can be determined, for example, by comparing the two sequences using freely available computer programs commonly employed for this purpose on the world wide web.

Examples of T3SS secretion signals and chaperone-binding domains are known in the art, see, e.g. Schmitz et al. Nat Methods 2009 6:500-2; which described the signals and domains of Shigella effectors. Additional examples are known in the art, e.g. Sory et al. PNAS 1995 92:11998-20002. It is contemplated that a T3SS signal may reduce the activity of the non-T3SS signal portion of the T3SS-compatible polypeptide once it is delivered to the target cell. Accordingly, in some embodiments, the T3SS-compatible polypeptide can comprise a cleavage site after the T3SS signal sequence. In some embodiments, the cleavage site is a site recognized by an endogenous component of the target cell, e.g. a calpain, sumo, and/or furin cleavage site. In some embodiments, instead of a cleavage site, the T3SS-compatible polypeptide can comprise an ubiquitin molecule after the T3SS signal sequence such that the ubiquitin molecule and the sequence N-terminal of it is removed from the remainder of the polypeptide by a eukaryotic target cell. In some embodiments, the first amino acid C-terminal of the ubiquitin molecule can be a methionine.

The T3SS-compatible polypeptide may be an antigen. An engineered microbial cell comprising a T3SS-compatible antigen polypeptide may be to a subject, e.g., orally.

In one aspect, described herein is a kit comprising an engineered microbial cell as described herein. In one aspect, described herein is a kit comprising an engineered microbial cell comprising a first nucleic acid sequence comprising genes encoding a functional type three secretion system (T3SS); and a second nucleic acid sequence encoding an T3SS-compatible polypeptide; wherein the engineered microbial cell is non-pathogenic with respect to a target cell.

Tumor-targeted bacteria, especially those derived from wild type samples, are typically capable of producing a persistent or even chronic infection without substantial infection-associated pathology and morbidity. That is, these bacteria seem to have evolved to avoid triggering a debilitating immune response in the host while at the same time establishing mid or long-term colonization of tissues, in the case of tumor targeting bacteria, tissues which may include necrotic regions. According to some evolutionary theories, the attenuated host response to these bacteria may result from a survival benefit for the host in permitting the colonization. Indeed, there are at least anecdotal reports of successful eradication of tumors by bacterial therapy, presumably due to development of a host immune response to the bacteria and the surrounding tumor tissues. The presence of the bacteria (or their antigens) appear to serve as an adjuvant for the tumor antigens, even if an acute debilitating inflammatory response to the bacteria themselves is not observed. This implies that bacteria derived from these strains can be pharmaceutically acceptable, for administration through various routes of administration.

Much research has been performed on bacterial therapies and bacterial delivery vectors. For example, tumor targeting bacteria offer tremendous potential advantages for the treatment of solid tumors, including the targeting from a distant inoculation site and the ability to express therapeutic agents directly within the tumor (Pawelek et al. 1997; Low et al. 1999). However, the primary shortcoming of tumor-targeted bacteria investigated in the human clinical trials (Salmonella strain VNP20009 also known as YS1646, and its derivative TAPET-CD; Toso et al. 2002; Nemunaitis et al. 2003) is that no significant antitumor activity has been observed, even in patients where the bacteria was documented to target the tumor. One method of increasing the ability of the bacteria to kill tumor cells is to engineer the bacteria to express conventional bacterial toxins (e.g., WO 2009/126189, WO 03/014380, WO/2005/018332, WO/2008/073148, US 2003/0059400 U.S. Pat. Nos. 7,452,531, 7,354,592, 6,962,696, 6,923,972, 6,863,894, 6,685,935, 6,475,482, 6,447,784, 6,190,657, 6,080,849, 8,241,623, 8,524,220, and 8,771,669).

Use of secreted proteins in live bacterial vectors has been demonstrated by several authors. Holland et al. (U.S. Pat. No. 5,143,830) have illustrated the use of fusions with the C-terminal portion of the hemolysin A (hlyA) gene, a member of the type I secretion system. When co-expressed in the presence of the hemolysin protein secretion channel (hlyBD) and a functional TolC, heterologous fusions are readily secreted from the bacteria. The type I secretion system that has been utilized most widely, and although it is currently considered the best system available, is thought to have limitations for delivery by attenuated bacteria (Hahn et al. 2003). Those limitations include the amount of protein secreted and the ability of the protein fused to it to interfere with secretion. Improvements of the type I secretion system have been demonstrated (Sugamata et al. 2005), using a modified hlyB, and by addition of rare codons to the hlyA gene (Gupta et al. 2008). Fusion to the gene ClyA (Galen et al. 2004) and Type III secretion proteins have also been used. Surface display has been used to export proteins outside of the bacteria. For example, fusion of the Lpp protein amino acids 1-9 with the transmembrane region B3-B7 of OmpA has been used for surface display (Samuelson et al. 2002). The autotransporter surface display has been described by Berthet et al., WO/2002/070645.

Other heterologous protein secretion systems utilizing the autotransporter family can be modulated to result in either surface display or complete release into the medium (see Henderson et al. 2004; Jose, 2006; Jose et al. 2005; Rutherford et al. 2006). For example, Veiga et al. 2003 and Klauser et al. 1990 demonstrated hybrid proteins containing the b-autotransporter domain of the immunoglobulin A (IgA) protease of Nisseria gonorrhea. Fusions to flagellar proteins have been demonstrated. The peptide, usually of 15 to 36 amino acids in length, is inserted into the central, hypervariable region of the FliC gene such as that from Salmonella muenchen (Verma et al. 1995; Wu et al. 1989; Cuadro et al. 2004; Newton et al. 1995). Multihybrid FliC insertions of up to 302 amino acids have also been prepared (Tanskanen et al. 2000).

Trimerization of antigens and functional proteins can be achieved using the T4 fibritin foldon trimerization sequence (Wei et al. 2008) and VASP tetramerization domains (Kühnel et al. 2004). The multimerization domains are used to create, bi-specific, tri-specific, and quatra-specific targeting agents, whereby each individual agent is expressed with a multimerization tag, each of which may have the same or separate targeting peptide, such that following expression, surface display, secretion and/or release, they form multimers with multiple targeting domains. Other secretion systems include C-terminal fusions to the protein YebF (Zhang et al. 2006), which is commercially available as a kit (pAES40; AthenaES, Baltimore, Md.). Fusions to OmsY and other proteins are also capable of secreting proteins into the medium (Zian et al. 2008). Other secretions systems usable according to the present invention include that of Kotzsch et al. 2011, or those described by Yoon et al. 2010. See, U.S. Pat. Nos. 5,470,719; 5,508,192, 5,824,502, 5,989,868, 6,083,715, 6,309,861, 6,455,279, 6,596,509, 6,596,510, 6,605,697, 6,642,027, 6,673,569, 6,828,121, 6,852,512, 6,861,403, 6,919,198, 6,921,659, 7,052,867, 7,056,732, 7,070,989, 7,094,579, 7,112,434, 7,105,327, 7,202,059, 7,291,325, 7,410,788, 7,491,528, 2004/0005695, 2006/0270043; 2007/0287171, 2008/0064062, 2008/0076157, 2008/0166757, 2008/0166764, 2008/0182295, 2008/0193974, 2008/0206814, 2008/0206818, 2008/0280346, 2009/0011995, 2008/0254511, EP0786009B1, EP0866132A2, EP1068339B1, EP1270730A1, EP1402036B1, EP1407052B1, WO2006/017929A1, WO2008/089132A2, and WO2009/021548A1.

Compositions described in accordance with various embodiments herein include, without limitation, Salmonella enterica serovar Typhimurium (“S. typhimurium”), Salmonella montevideo, Salmonella enterica serovar Typhi (“S. typhi”), Salmonella enterica serovar Paratyphi A, Paratyphi B (“S. paratyphi 13”), Salmonella enterica serovar Paratyphi C (“S. paratyphi C”), Salmonella enterica serovar Hadar (“S. hadar”), Salmonella enterica serovar Enteriditis (“S. enteriditis”), Salmonella enterica serovar Kentucky (“S. kentucky”), Salmonella enterica serovar Infantis (“S. infantis”), Salmonella enterica serovar Pullorum (“S. pullorum”), Salmonella enterica serovar Gallinarum (“S. gallinarum”), Salmonella enterica serovar Muenchen (“S. muenchen”), Salmonella enterica serovar Anaturn (“S. anatum”), Salmonella enterica serovar Dublin (“S. dublin”), Salmonella enterica serovar Derby (“S. derby”), Salmonella enterica serovar Choleraesuis var. kunzendorf (“S. cholerae kunzendorf”), and Salmonella enterica serovar minnesota (S. minnesota).

By way of example, live bacteria in accordance with aspects of the invention may include known strains of S. enterica serovar Typhimurium (S. typhimurium) and S. enterica serovar Typhi (S. typhi) which are further modified as provided by various embodiments of the invention. Such Strains include Ty21a, CMV906, CMV908, CMV906-htr, CMV908-htr, Ty800, aroA−/serC−, holavax, M01ZH09, VNP20009. These strains contain defined mutations within specific serotypes of bacteria. The technology also includes the use of these same (or different) mutational combinations contained within alternate serotypes or strains in order to avoid immune reactions which may occur in subsequent administrations. For example, S. Typhimurium, S. montevideo, and S. typhi which have non-overlapping O-antigen presentation (e.g., S. typhimurium is O—1, 4, 5, 12 and S. typhi is Vi, S. montevideo is O—6, 7) may be used. Thus, for example, S. typhimurium is a suitable serotype for a first administration and another serotype such as S. typhi or S. montevideo are used for a second administration and third administration. Likewise, the flagellar antigens are also selected for non-overlapping antigenicity between different administrations. The flagellar antigen may be H1 or H2 or no flagellar antigen, which, when combined with the three different O-antigen serotypes, provides three completely different antigenic profiles.

Other genus and species of bacteria as discussed below, are also encompassed. Typically, the bacteria are naturally probiotic or attenuated, such that morbidity or mortality risk as a result of infection is low or absent. Further, the bacteria preferably do not induce TNFα, and thus are lipid A deficient, and avoid septic shock risk. Likewise, various other genes or gene products associated with pathogenicity are also reduced or absent, unless these defeat viability of the bacteria or their ability to present the desired antigen to the immune system.

Winter et al. (2017), earlier work of the inventors suggest that attenuated Salmonella enterica species are attractive as vaccine vectors due to their potential to induce both local (mucosal) and systemic immune responses. To facilitate stimulation of immune responses, type III secretion systems (T3SS) of Salmonella can be employed to deliver heterologous antigens to antigen-presenting cells. The genome of S. enterica contains two loci termed Salmonella pathogenicity island 1 and 2 (SPI-I and SPI-II) that encode distinct T3SS that translocate effector proteins at the different stages of Salmonella infection. While these secretion systems have been exploited previously to deliver foreign antigens in Salmonella-based vaccine development efforts, the distinct spatial and temporal functions of the SPI-I and SPI-II systems on immune responses, particularly in terms of mucosal immunity, have yet to be systemically investigated. Proposed antigenic targets are the C-terminal receptor binding domains (RBDs) of Clostridium difficile toxins A and B (TcdA, TcdB). Anti-RBD antibodies have been shown to protect against C. difficile infection in both animal models and humans. A panel of 13 vaccine candidates has been developed based on a well-characterized, attenuated S. typhimurium strain (YS1646) that express the RBDs of either TcdA or TcdB using different SPI-I and SPI-II promoters and secretory signals. Western Blot and immunofluorescence results show that expression of these antigens is variable in vitro, both when the bacteria is grown in LB broth and upon invasion of a mouse macrophage cell line (RAW264.7).

Preliminary data in a mouse vaccination model (3 doses of 10⁹ bacteria by gavage either every other day or every 2 weeks) suggest that several of these vaccine candidate that exploit different SPI-I and SPI-II T3SS promoters and secretory signals elicit systemic immune responses at least (IgG by ELISA). The vaccine schedule was not optimized to find the construct that elicit both systemic and mucosal immunity (serum IgG, stool fluid IgA, cellular responses). Thus, while it was shown that YS1646 could be used to produce vaccine candidates with TcdA and TcdB antigens secreted by the SPI-I or SPI-II T3SS system, and that these could raise IgG immune responses in mice, the existence of IgA response or protective immunity was not demonstrated, and required seven doses of bacteria. See also, Wang et al. 2018.

See also, U.S. Pat. No. 6,548,287, and EP0973911. See also, US 20140256922; 20120108640; 20110318308; 20090215754; 20090169517; 20070298012; 20070110752; 20070004666; 20060115483; 20060104955; 20060089350; 20060025387; 20050267103; 20050249706; 20050112642; 20050009750; 20040229338; 20040219169; 20040058849; 20030143676; 20030113293; 20030031628; 20030022835; 20020151063; 20140220661; 20140212396; 20140186401; 20140178341; 20140155343; 20140093885; 20130330824; 20130295054; 20130209405; 20130130292; 20120164687; 20120142080; 20120128594; 20120093773; 20120020883; 20110275585; 20110111496; 20110111481; 20100239546; 20100189691; 20100136048; 20100135973; 20100135961; 20100092438; 20090300779; 20090180955; 20090175829; 20090123426; 20090053186; 20080311081; 20080124355; 20080038296; 20070110721; 20070104689; 20060083716; 20050026866; 20050008618; 20040202663; 20050255088; 20030109026; 20020026655; 20110223241; 20070009489; 20050036987; 20030170276; 20140148582; 20130345114; 20130287810; 20130164380; 20130164307; 20130078275; 20120225454; 20120177682; 20120148601; 20120144509; 20120083587; 20120021517; 20110274719; 20110268661; 20110165680; 20110091493; 20110027349; 20100172976; 20090317404; 20090220540; 20090123382; 20090117049; 20090117048; 20090117047; 20090068226; 20080249013; 20080206284; 20070202591; 20070191262; 20070134264; 20060127408; 20060057152; 20050118193; 20050069491; 20050064526; 20040234455; 20040202648; 20040054142; 20030170211; 20030059400; 20030036644; 20030009015; 20030008839; 20020176848; 20020102242; 20140205538; 20140112951; 20140086950; 20120244621; 20120189572; 20110104196; 20100233195; 20090208534; 20090136542; 20090028890; 20080260769; 20080187520; 20070031382; 20060140975; 20050214318; 20050214317; 20050112140; 20050112139; 20040266003; 20040115174; 20040009936; 20030153527; 20030125278; 20030045492; U.S. Pat. Nos. 8,828,681; 8,822,194; 8,784,836; 8,771,669; 8,734,779; 8,722,668; 8,715,641; 8,703,153; 8,685,939; 8,663,634; 8,647,642; 8,642,257; 8,623,350; 8,604,178; 8,591,862; 8,586,022; 8,568,707; 8,551,471; 8,524,220; 8,440,207; 8,357,486; 8,343,509; 8,323,959; 8,282,919; 8,241,623; 8,221,769; 8,198,430; 8,137,904; 8,066,987; 8,021,662; 8,008,283; 7,998,461; 7,955,600; 7,939,319; 7,915,218; 7,887,816; 7,842,290; 7,820,184; 7,803,531; 7,790,177; 7,786,288; 7,763,420; 7,754,221; 7,740,835; 7,736,898; 7,718,180; 7,700,104; 7,691,383; 7,687,474; 7,662,398; 7,611,883; 7,611,712; 7,588,771; 7,588,767; 7,514,089; 7,470,667; 7,452,531; 7,404,963; 7,393,525; 7,354,592; 7,344,710; 7,247,296; 7,195,757; 7,125,718; 7,084,105; 7,083,791; 7,015,027; 6,962,696; 6,923,972; 6,916,918; 6,863,894; 6,770,632; 6,685,935; 6,682,729; 6,506,550; 6,500,419; 6,475,482; 6,447,784; 6,207,648; 6,190,657; 6,150,170; 6,080,849; 6,030,624; and 5,877,159.

Novel strains are also encompassed that are, for example, attenuated in virulence by mutations in a variety of metabolic and structural genes. The invention therefore may provide a live composition for treating infection comprising a live attenuated bacterium that is a serovar of Salmonella enterica comprising an attenuating mutation in a genetic locus of the chromosome of said bacterium that attenuates virulence of said bacterium and wherein said attenuating mutation is the Suwwan deletion (Murray et al. 2004) or combinations with other known attenuating mutations. Other attenuating mutation useful in the Salmonella bacterial strains described herein may be in a genetic locus selected from the group consisting of phoP, phoQ, edt, cya, crp, poxA, rpoS, htrA, nuoG, pmi, pabA, pts, damA, pur, purA, purB, purI, purF, zwf, aroA, aroB, aroC, aroD, serC, gua, cadA, rfc, rjb, rfa, ompR, msbB, leucine and arginine, and combinations thereof. Strains of Salmonella deleted in stn are particularly preferred.

Attenuated gram-positive bacteria are also available as delivery vectors. For example, Staphylococcus epidermidis, group B Streptococcus including S. agalaciae, and Listeria species including L. monocytogenes may be employed. It is known to those skilled in the art that variations in molecular biology techniques such as use of gram-positive origins of replication, gram-positive signal sequences and gram-positive promoters and filamentous phage (e.g., phage B5; Chopin et al., 2002 J. Bacteriol. 184: 2030-2033, described further below) may be employed and substituted as needed. Other bacterial strains may also be encompassed, including non-pathogenic bacteria of the gut skin (such as Staphylococcus epidermidis, Proprionibacteria) and other body locations known as the human microbiome (Grice et al. 2012; Spor et al. 2011) such as E. coli strains, Bacteriodies, Bifidobacterium and Bacillus, attenuated pathogenic strains of E. coli including enteropathogenic and uropathogenic isolates, Enterococcus sp. and Serratia sp. as well as attenuated Neisseria sp., Shigella sp., Staphylococcus sp., Staphylococcus carnosis, Yersinia sp., Streptococcus sp. and Listeria sp. including L. monocytogenes. Bacteria of low pathogenic potential to humans and other mammals or birds or wild animals, pets and livestock, such as insect pathogenic Xenorhabdus sp., Photorhabdus sp. and human wound Photorhabdus (Xenorhabdus) are also encompassed. Probiotic strains of bacteria are also encompassed, including Lactobacillus sp. (e.g., Lactobacillus acidophilus, Lactobacillus salivarius) Lactococcus sp., (e.g., Lactococcus lactis, Lactococcus casei) Leuconostoc sp., Pediococcus sp., Streptococcus sp. (e.g., S. salivariu, S. thermophilus), Bacillus sp., Bifidobacterium sp., Bacteroides sp., and Escherichia coli such as the 1917 Nissel strain.

It is known to those skilled in the art that minor variations in molecular biology techniques such as use of gram-positive origins of replication, gram-positive signal sequences gram-positive promoters (e.g., Lactococcus expression, Mohamadzadeh et al. 2009) may be used and substituted as needed. The bacteria may be further modified to be internalized into the host cell (Guimaraes et al. 2006; Innocentin et al. 2009).

Recently developed approaches to delivery of therapeutic molecules (U.S. Pat. Nos. 8,241,623; 8,524,220; and 8,771,669) have coupled a protease sensitive therapeutic molecule with co-expression of protease inhibitors.

A fusion with the Pseudomonas ice nucleation protein (INP) wherein the N- and C-terminus of INP with an internal deletion consisting of the first 308 amino acids is followed by the mature sequence of the protein to be displayed (Jung et al. 1998; Kim et al., 2000; Part:BBa_K811003 from www.iGEM.org; WO2005005630).

Clostridium difficile causes one of the most important nosocomial infections in the world (Heimann et al. 2018; Rupnik et al. 2009). Clinically-apparent C. difficile infection (CDI) is most often caused by antibiotics that disrupt the gastrointestinal microflora, permitting overgrowth of C. difficile and production of toxins A and B (TcdA and TcdB). TcdA, an enterotoxin, and TcdB, a cytotoxin, represent two of the principal virulence factors of C. difficile (Ananthakrishnan et al. 2010) and both are expressed in most clinical isolates. Together, they disrupt the actin cytoskeleton of enterocytes in the gastrointestinal epithelium, resulting in fluid accumulation, inflammation and severe tissue damage (Carter et al. 2010). Some strains produce an additional toxin called the binary toxin or CDT (Reigadas et al. 2016).

The prevalence and severity of CDI has increased significantly in most countries over the past 2-3 decades (Rupnik et al. 2009; Wiegand et al. 2012). More than 370,000 cases occur every year in North America alone with an estimated total cost exceeding 6 billion dollars (Zhang et al. 2016). Currently, antibiotics are routinely recommended for the treatment of CDI (e.g., metronidazole, vancomycin, fidaxomicin alone or in combination) despite the irony of treating a disease caused by antibiotics with further antibiotics. Recurrent CDI after treatment and severe CDI are significant problems poorly-responsive to antibiotics (Surawicz et al. 2011). Effective control of CDI is complicated by asymptomatic carriage, including post-treatment, and by spores that can persist in the environment for prolonged periods.

Preventing CDI-associated morbidity and mortality requires new approaches including the development of vaccines. Clostridium difficile is non-invasive, so CDI is largely a toxin-mediated disease. Indeed, the outcome of CDI in both animal models and humans is strongly correlated with the host antibody response to TcdA and/or TcdB (Greenberg et al. 2012). These toxins have therefore been a major focus of both active and passive immunotherapeutic strategies and several toxin-based vaccines have advanced to phase II/III clinical trials (Bruxelle et al. 2018). Of particular interest to the current studies, both pre-clinical (Baliban et al. 2014; Ibarra et al. 2009) and clinical-stage work (Bézay et al. 2016) support the idea of targeting the RBDs of these toxins. Whether whole protein, toxoid or RBD however, most of the effort to elicit anti-toxin responses has focused on peripheral, intramuscular (IM), administration of these antigens. Furthermore, as is typical for non-living vaccines, almost all of these candidates have required an adjuvant and multiple doses over several months to achieve an adequate immune response (Bruxelle et al. 2018).

Salmonella are also encompassed that are, for example, attenuated in virulence by mutations in a variety of metabolic and structural genes. The technology therefore may provide a live composition for treating infection comprising a live attenuated bacterium that is a serovar of Salmonella enterica comprising an attenuating mutation in a genetic locus of the chromosome of said bacterium that attenuates virulence of said bacterium and wherein said attenuating mutation is a combinations of other known attenuating mutations. Other attenuating mutation useful in the Salmonella bacterial strains described herein may be in a genetic locus selected from the group consisting of phoP, phoQ, edt, cya, crp, poxA, rpoS, htrA, nuoG, pmi, pabA, pts, damA, met, cys, pur, purA, purB, purI, purF, leu, ilv, arg, lys, zwf, aroA, aroB, aroC, aroD, serC, gua, cadA, rfc, rjb, rfa, ompR, msbB, pfkAB, crr, glk, ptsG, ptsHI, manXYZ and combinations thereof. The strain may also contain a mutation known as “Suwwan”, which is an approximately 100 kB deletion between two IS200 elements. The strain may also carry a defective thioredoxin gene (trxA⁻; which may be used in combination with a TrxA fusion), a defective glutathione oxidoreductase (gor⁻) and optionally, overexpress a protein disulfide bond isomerase (DsbA). The strain may also be engineered to express invasion and/or escape genes tlyA, tlyC patI and pld from Rickettsia, whereby the bacteria exhibit enhanced invasion and/or escape from the phagolysosome (Witworth et al. 2005), thereby enhancing the activity of the effector genes described below. The strain may also be engineered to be deleted in an avirulence (anti-virulence) gene, such as zirTS, grvA and/or pcgL, or express the E. coli lac repressor, which is also an avirulence gene in order to compensate for over-attenuation. The strain may also express SlyA, a known transcriptional activator. In a preferred embodiment, the Salmonella strains are msbB mutants (msbB⁻). In a more preferred embodiment, the strains are msbB− and Suwwan. In a more preferred embodiment the strains are msbB⁻, Suwwan and zwf⁻. Zwf has recently been shown to provide resistance to CO2, acidic pH and osmolarity (Karsten et al. 2009). Use of the msbB zwf genetic combination is also particularly preferred for use in combination with administered carbogen (an oxygen carbon dioxide mixture that may enhance delivery of therapeutic agents to a tumor). In a more preferred embodiment, the strains are msbB⁻, Suwwan, zwf⁻ and trxA⁻. In a most preferred embodiment, the strains are msbB⁻, Suwwan, zwf⁻, trxA⁻ and gor⁻.

The technology also provides, according to one embodiment, a process for preparing genetically stable therapeutic bacterial strains comprising genetically engineering the therapeutic genes of interest into a bacterially codon optimized expression sequence within a bacterial plasmid expression vector, endogenous virulence (VIR) plasmid (of Salmonella sp.), or chromosomal localization expression vector for any of the deleted genes or IS200 genes, defective phage or intergenic regions within the strain and further containing engineered restriction endonuclease sites such that the bacterially codon optimized expression gene contains subcomponents which are easily and rapidly exchangeable, and the bacterial strains so produced.

The present technology provides, for example, and without limitation, live bacterial compositions that are genetically engineered to express one or more protease inhibitors combined with antigens.

According to various embodiments, the technology provides pharmaceutical compositions comprising pharmaceutically acceptable carriers and one or more bacterial mutants. The technology also provides pharmaceutical compositions comprising pharmaceutically acceptable carriers and one or more bacterial mutants comprising nucleotide sequences encoding one or more peptides. Preferably, the bacterial mutants are attenuated by introducing one or more mutations in one or more genes in the lipopolysaccharide (LPS) biosynthetic pathway (for gram-negative bacteria), and optionally one or more mutations to auxotrophy for one or more nutrients or metabolites.

In one embodiment, a pharmaceutical composition comprises a pharmaceutically acceptable carrier and one or more bacterial mutants, wherein said attenuated bacterial mutants are facultative anaerobes or facultative aerobes. In another embodiment, a pharmaceutical composition comprises a pharmaceutically acceptable carrier and one or more attenuated bacterial mutants, wherein said attenuated bacterial mutants are facultative anaerobes or facultative aerobes. In one embodiment, a pharmaceutical composition comprises a pharmaceutically acceptable carrier and one or more bacterial mutants, wherein said attenuated bacterial mutants are facultative anaerobes or facultative aerobes. In another embodiment, a pharmaceutical composition comprises a pharmaceutically acceptable carrier and one or more attenuated bacterial mutants, wherein said attenuated bacterial mutants are facultative anaerobes or facultative aerobes.

A pharmaceutically effective dosage form may comprise between about 10⁵ to 10¹² live bacteria, within a lyophilized medium for oral administration. In some embodiments, about 10⁹ live bacteria are administered.

Pharmaceutically Acceptable Formulations

Pharmaceutically acceptable formulations may be provided for delivery by other various routes e.g. by intramuscular injection, subcutaneous delivery, by intranasal delivery (e.g. WO 00/47222, U.S. Pat. No. 6,635,246), intradermal delivery (e.g. WO02/074336, WO02/067983, WO02/087494, WO02/0832149 WO04/016281) by transdermal delivery, by transcutaneous delivery, by topical routes, etc. Injection may involve a needle (including a microneedle), or may be needle-free. See, e.g., U.S. Pat. Nos. 7,452,531, 7,354,592, 6,962,696, 6,923,972, 6,863,894, 6,685,935, 6,475,482, 6,447,784, 6,190,657, 6,080,849 and US Pub. 2003/0059400.

Bacterial vector vaccines are known, and similar techniques may be used for the present bacteria as for bacterial vaccine vectors (U.S. Pat. No. 6,500,419, Curtiss 1989; and Mims 1993). These known vaccines can enter the host, either orally, intranasally or parenterally. Once gaining access to the host, the bacterial vector vaccines express an engineered prokaryotic expression cassette contained therein that encodes a foreign antigen(s). Foreign antigens can be any protein (or part of a protein) or combination thereof from a bacterial, viral, or parasitic pathogen that has vaccine properties (New Generation Vaccines; Hilleman 1994; Formal et al 1981; Gonzalez et al. 1994; Stevenson et al, 1985; Aggarwal et al 1990; Hone et al 1988; Flynn et al. 1990; Walker et al 1992; Cardenas et al. 1993; Curtiss et al. 1994; Simonet et al. 1994; Charbit et al. 1993; Turner et al. 1993; Schodel et al. 1994; Schodel et al. 1990; Stabel et al. 1991; Brown 1987; Doggett et al. 1993; Brett et al. 1993; Yang et al. 1990; Gao et al. 1992; and Chatfield et al. 1992). Delivery of the foreign antigen to the host tissue using bacterial vector vaccines results in host immune responses against the foreign antigen, which provide protection against the pathogen from which the foreign antigen originates (Mims 1987; New Generation Vaccines). See also (Formal et al. 1981); Wick et al. 1994); Hone et al. 1991; Tacket et al. 1992; Hone et al. 1992; Chatfield et al. 1992; Tacket et al. 1992; van Damme et al. 1992 (Yersinia pestis), Noriega et al. 1994 (Shigella spp), Levine et al. 1994 (Vibrio cholerae); Lagranderie et al 1993; Flynn 1994 (Mycobacterium strain BCG), Schafer et al. 1992 (Listeria monocytogenes)).

The bacteria are generally administered along with a pharmaceutically acceptable carrier and/or diluent. The particular pharmaceutically acceptable carrier and/or diluent employed is not critical to the present invention unless otherwise specific herein (or in a respective incorporated referenced relevant to the issue). Examples of diluents include a phosphate buffered saline, buffer for buffering against gastric acid in the stomach, such as citrate buffer (pH 7.0) containing sucrose, bicarbonate buffer (pH 7.0) alone (Levine et al 1987), or bicarbonate buffer (pH 7.0) containing ascorbic acid, lactose, and optionally aspartame (Levine et al, Lancet, 11:467-470 (1988)). Examples of carriers include proteins, e.g., as found in skim milk, sugars, e.g., sucrose, or polyvinylpyrrolidone. Typically, these carriers would be used at a concentration of about 0.1-30% (w/v) but preferably at a range of 1-10% (w/v).

Set forth below are other pharmaceutically acceptable carriers or diluents which may be used for delivery specific routes. Any such carrier or diluent can be used for administration of the bacteria of the invention, so long as the bacteria are still capable of invading a target cell. In vitro or in vivo tests for invasiveness can be performed to determine appropriate diluents and carriers. The compositions of the invention can be formulated for a variety of types of administration, including systemic and topical or localized administration. Lyophilized forms are also included, so long as the bacteria are invasive upon contact with a target cell or upon administration to the subject. Techniques and formulations generally may be found in Remington's Pharmaceutical Sciences, Meade Publishing Co., Easton, Pa. For systemic administration, injection is preferred, including intramuscular, intravenous, intraperitoneal, and subcutaneous. For injection, the composition, e.g., bacteria, of the invention can be formulated in liquid solutions, preferably in physiologically compatible buffers such as Hank's solution or Ringer's solution.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner.

For administration by inhalation, the pharmaceutical compositions for use according to the present invention are conveniently delivered in the form of an aerosol spray presentation from pressurized packs or a nebulizer, with the use of a suitable propellant, e.g., a hydrofluorocarbon (HFC), carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the composition, e.g., bacteria, and a suitable powder base such as lactose or starch.

The pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. See also U.S. Pat. No. 6,962,696.

The present invention provides a pharmaceutical composition comprising a pharmaceutically acceptable carrier and an attenuated tumor-targeted bacteria comprising one or more nucleic acid molecules encoding one or more primary effector molecules operably linked to one or more appropriate promoters. The present invention provides a pharmaceutical composition comprising a pharmaceutically acceptable carrier and an attenuated tumor-targeted bacteria comprising one or more nucleic acid molecules encoding one or more primary effector molecules and one or more secondary effector molecules operably linked to one or more appropriate promoters.

The present invention provides a pharmaceutical composition comprising a pharmaceutically acceptable carrier and a bacterium.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, olive oil, and the like. Saline is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic attenuated tumor-targeted bacteria, in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a suspending agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the pharmaceutical composition of the invention which will be effective in the vaccination of a subject can be determined by standard clinical techniques. In addition, in vitro assays may optionally be employed to help identify optimal dosage ranges. The precise dose to be employed in the formulation will also depend on the route of administration, and should be decided according to the judgment of the practitioner and each patient's circumstances. However, suitable dosage ranges are generally from about 1.0 cfu/kg to about 1×10¹⁰ cfu/kg; optionally from about 1.0 cfu/kg to about 1×10⁸ cfu/kg; optionally from about 1×10² cfu/kg to about 1×10⁸ cfu/kg; optionally from about 1 10⁴ cfu/kg to about 1×10⁸ cfu/kg; and optionally from about 1×10⁴ cfu/kg to about 1×10¹⁰ cfu/kg (cfu=colony forming unit). Effective doses may be extrapolated from dose-response curves derived from in vitro or animal model test systems.

Various delivery systems are known and can be used to administer a pharmaceutical composition of the present invention. Methods of introduction include but are not limited to intradermal, intramuscular, intraperitoneal, intravenous, subcutaneous, intranasal, and oral routes. The compositions may be administered by any convenient route, for example by infusion or bolus injection, by absorption through epithelial or mucocutaneous linings (e.g., oral mucosa, rectal and intestinal-mucosa, etc.) and may be administered together with other biologically active agents. Administration can be systemic or local. Pulmonary administration can also be employed, e.g., by use of an inhaler or nebulizer, and formulation with an aerosolizing agent.

The invention also provides a pharmaceutical pack or kit comprising one or more containers filled with one or more of the ingredients of the pharmaceutical compositions of the invention. Optionally associated with such container(s) can be a notice in the form prescribed by governmental agency regulating the manufacture, use or sale of pharmaceuticals or biological products, which notice reflects approval by the agency of manufacture, use or sale for human administration.

See, U.S. Pat. Nos. 4,190,495; 4,888,170; 4,968,619; 5,066,596; 5,098,998; 5,294,441; 5,330,753; 5,387,744; 5,424,065; 5,468,485; 5,527,678; 5,627,067; 5,628,996; 5,643,771; 5,654,184; 5,656,488; 5,662,905; 5,672,345; 5,679,880; 5,686,079; 5,695,983; 5,717,071; 5,731,196; 5,736,367; 5,747,028; 5,770,214; 5,773,007; 5,811,105; 5,824,538; 5,830,702; 58375095837541; 5,840,483; 5,843,426; 5,855,879; 5,855,880; 5,869,066; 5,874,088; 5,877,159; 5,888,799; 6,024,961; 6,051,416; 6,077,678; 6,080,849; 6,100,388; 6,129,917; 6,130,082; 6,150,170; 6,153,203; 6,177,083; 6,190,657; 6,207,167; 6,245,338; 6,254,875; 6,284,477; 6,294,655; 6,337,072; 6,339,141; 6,365,163; 6,365,723; 6,365,726; 6,372,892; 6,383,496; 6,410,012; 6,413,523; 6,426,191; 6,444,445; 6,447,784; 6,471,964; 6,475,482; 6,495,661; 6,500,419; 6,506,550; 6,511,666; 6,531,313; 6,537,558; 6,541,623; 6,566,121; 6,593,147; 6,599,509; 6,610,300; 6,610,529; 6,653,128; 6,682,729; 6,685,935; 6,719,980; 6,737,521; 6,749,831; 6,752,994; 6,780,405; 6,825,028; 6,855,814; 6,863,894; 6,872,547; 6,887,483; 6,905,691; 6,913,753; 6,916,478; 6,923,958; 6,923,972; 6,962,696; 6,992,237; 6,994,860; 7,005,129; 7,018,835; 7,026,155; 7,045,336; 7,056,700; 7,063,850; 7,083,794; 7,094,410; 7,115,269; 7,144,580; 7,144,982; 7,183,105; 7,195,757; 7,226,588; 7,235,234; 7,264,812; 7,279,464; 7,341,725; 7,341,841; 7,341,860; 7,354,592; 7,393,525; 7,407,790; 7,425,438; 7,452,531; 7,459,161; 7,473,247; 7,510,717; 7,514,089; 7,514,415; 7,531,723; 7,541,043; 7,569,219; 7,569,552; 7,569,682; 7,588,767; 7,588,771; 7,601,804; 7,622,107; 7,625,572; 7,657,380; 7,662,398; 7,666,656; 7,691,393; 7,695,725; 7,700,091; 7,700,104; 7,718,179; 7,732,187; 7,754,221; 7,758,876; 7,763,420; 7,772,386; 7,776,527; 7,794,734; 7,803,531; 7,803,990; 7,807,184; 7,807,456; 7,820,184; 7,829,104; 7,833,775; 7,842,289; 7,842,290; 7,850,958; 7,850,970; 7,871,604; 7,871,815; 7,871,816; 7,887,816; 7,919,081; 7,927,606; 7,930,107; 7,951,386; 7,951,786; 7,955,600; 7,960,518; 7,972,604; 7,985,573; 7,993,651; 8,012,466; 8,021,662; 8,021,848; 8,034,359; 8,043,857; 8,048,428; 8,049,000; 8,053,181; 8,053,421; 8,066,987; 8,071,084; 8,071,319; 8,076,099; 8,101,396; 8,114,409; 8,114,414; 8,124,068; 8,124,408; 8,133,493; 8,137,904; 8,147,820; 8,168,421; 8,173,773; 8,187,610; 8,202,516; 8,207,228; 8,211,431; 8,221,769; 8,227,584; 8,241,623; 8,241,631; 8,241,637; 8,257,713; 8,273,361; 8,287,883; 8,288,359; 8,318,661; 8,323,668; 8,323,959; 8,329,685; 8,337,832; 8,337,861; 8,343,509; 8,343,512; 8,349,586; 8,357,486; 8,357,533; 8,361,707; 8,367,055; 8,399,618; 8,440,207; 8,445,254; 8,445,426; 8,445,662; 8,460,666; 8,465,755; 8,470,551; 8,481,055; 8,501,198; 8,524,220; 8,551,497; 8,557,789; 8,568,707; 8,580,280; 8,586,022; 8,591,862; 8,609,114; 8,623,350; 8,628,776; 8,632,783; 8,633,305; 8,642,257; 8,642,656; 8,647,642; 8,658,350; 8,663,940; 8,669,355; 8,673,311; 8,679,505; 8,703,153; 8,715,929; 8,716,254; 8,716,343; 8,722,064; 8,748,150; 8,758,766; 8,771,669; 8,772,013; 8,778,683; 8,784,829; 8,784,836; 8,790,909; 8,840,908; 8,853,382; 8,859,256; 8,877,212; 8,883,147; 8,889,121; 8,889,150; 8,895,062; 8,916,372; 8,926,993; 8,937,074; 8,951,531; 8,956,618; 8,956,621; 8,956,859; 8,961,989; 8,980,279; 8,992,943; 9,005,665; 9,011,870; 9,012,213; 9,017,986; 9,023,635; 9,040,059; 9,040,233; 9,045,528; 9,045,742; 9,050,285; 9,050,319; 9,051,574; 9,056,909; 9,062,297; 9,068,187; 9,107,864; 9,140,698; 9,161,974; 9,163,219; 9,169,302; 9,173,930; 9,173,935; 9,173,936; 9,180,183; 9,181,546; 9,198,960; 9,200,251; 9,200,289; 9,205,142; 9,220,764; 9,248,177; 9,255,149; 9,255,283; 9,265,804; 9,267,108; 9,289,481; 9,297,015; 9,303,264; 9,309,493; 9,315,817; 9,320,787; 9,320,788; 9,333,251; 9,339,533; 9,358,283; 9,364,528; 9,365,625; 9,376,686; 9,408,880; 9,415,077; 9,415,098; 9,421,252; 9,428,572; 9,441,204; 9,453,227; 9,457,074; 9,457,077; 9,463,238; 9,474,831; 9,480,740; 9,481,884; 9,481,888; 9,486,513; 9,487,577; 9,492,534; 9,499,606; 9,504,750; 9,506,922; 9,526,778; 9,529,005; 9,539,313; 9,540,407; 9,546,199; 9,549,956; 9,556,442; 9,561,270; 9,562,080; 9,562,837; 9,566,321; 9,566,322; 9,567,375; 9,580,478; 9,580,718; 9,592,283; 9,593,339; 9,597,379; 9,598,697; 9,603,799; 9,610,342; 9,616,114; 9,622,486; 9,636,386; 9,642,881; 9,642,904; 9,649,345; 9,651,559; 9,655,815; 9,657,085; 9,657,327; 9,662,385; 9,663,758; 9,670,270; 9,695,229; 9,714,426; 9,717,782; 9,730,996; 9,737,592; 9,737,601; 9,739,773; 9,750,802; 9,758,572; 9,764,021; 9,775,896; 9,795,641; 9,796,762; 9,801,930; 9,808,517; 9,814,772; 9,827,305; 9,844,592; 9,845,342; 9,855,336; 9,856,311; 9,867,785; 9,872,898; 9,878,023; 9,878,024; 9,878,043; 9,884,108; 9,885,051; 9,889,165; 9,901,082; 9,907,755; 9,907,845; 9,913,893; 9,925,257; 9,950,063; 9,986,724; 9,987,355; 9,994,809; 9,999,660; 20010014673; 20020025325; 20020028215; 20020044938; 20020068068; 20020076417; 20020077272; 20020081317; 20020086032; 20020086332; 20020090376; 20020132789; 20020146430; 20020151462; 20020156009; 20020176848; 20030017162; 20030023075; 20030045492; 20030065039; 20030068328; 2003010100100; 20030108562; 20030108957; 20030124516; 20030125278; 20030130827; 20030152589; 20030153527; 20030157637; 20030166099; 20030166279; 20030170211; 20030170613; 20030176377; 20030180260; 20030180304; 20030180320; 20030185802; 20030186908; 20030190601; 20030190683; 20030190749; 20030194714; 20030194755; 20030194798; 20030198995; 20030198996; 20030199005; 20030199088; 20030199089; 20030202937; 20030203411; 20030203481; 20030207833; 20030211086; 20030211103; 20030211461; 20030211476; 20030211599; 20030219408; 20030219888; 20030224369; 20030224444; 20030232335; 20030235577; 20040005700; 20040009540; 20040009936; 20040013658; 20040013689; 20040023310; 20040033539; 20040052817; 20040053209; 20040077067; 20040121307; 20040121474; 20040126871; 20040131641; 20040132678; 20040137003; 20040156865; 20040192631; 20040202663; 20040213804; 20040228877; 20040229338; 20040237147; 20040258703; 20040258707; 20040265337; 20050008618; 20050010032; 20050026866; 20050042755; 20050048076; 20050075298; 20050106151; 20050106176; 20050129711; 20050163791; 20050175630; 20050180985; 20050222057; 20050229274; 20050232937; 20050233408; 20050249706; 20050249752; 20050255125; 20050271643; 20050271683; 20050281841; 20050287123; 20060018877; 20060019239; 20060074039; 20060078572; 20060083716; 20060115494; 2006012145; 20060121054; 20060140971; 20060140975; 20060147418; 20060147461; 20060153875; 20060171960; 20060182754; 20060189792; 20060193874; 20060233835; 20060240494; 20060246083; 20060257415; 20060269570; 20070031382; 20070031458; 20070037225; 20070048331; 20070059323; 20070104733; 20070104736; 20070110717; 20070116725; 20070122881; 20070128216; 20070134214; 20070134272; 20070141082; 20070154495; 20070169226; 20070189982; 20070258901; 20070281328; 20070286874; 20070298012; 20080008718; 20080020441; 20080038296; 20080063655; 20080095794; 20080107653; 20080112974; 20080124355; 20080131466; 20080138359; 20080181892; 20080188436; 20080193373; 20080213308; 20080241179; 20080241858; 20080254058; 20080261869; 20080286852; 20080311081; 20080317742; 20090017000; 20090017048; 20090028892; 20090053186; 20090074816; 20090081250; 20090081257; 20090104204; 20090117151; 20090117152; 20090123426; 20090142310; 20090148473; 20090169517; 20090169562; 20090180987; 20090181078; 20090196887; 20090214476; 20090227013; 20090253778; 20090263414; 20090263418; 20090285844; 20090297552; 20090297561; 20090304750; 20090305398; 20090324503; 20090324576; 20090324638; 20090324641; 20100047286; 20100055082; 20100055127; 20100068214; 20100092438; 20100092518; 20100099600; 20100129406; 20100135961; 20100136048; 20100136055; 20100136058; 20100137192; 20100166786; 20100166800; 20100172938; 20100172976; 20100189691; 20100196524; 20100209446; 20100226891; 20100226931; 20100226941; 20100233212; 20100233213; 20100239546; 20100272748; 20100272759; 20100285592; 20100291148; 20100297184; 20100297740; 20100303862; 20100310602; 20100322957; 20110008389; 20110014274; 20110020401; 20110021416; 20110052628; 20110059126; 20110064723; 20110064766; 20110070290; 20110086059; 20110104186; 20110110979; 2010111481; 20110111496; 20110123565; 20110183342; 20110195093; 20110200631; 20110201092; 20110201676; 20110206694; 20110209228; 20110212090; 20110213129; 20110217323; 20110243992; 20110256214; 20110268739; 20110275585; 20110281330; 20110287046; 20110293662; 20110312020; 20120003298; 20120009247; 20120014881; 20120027811; 20120036589; 20120039931; 20120039994; 20120058142; 20120071545; 20120077206; 20120093773; 20120093850; 20120093865; 20120100177; 20120107340; 20120115223; 20120121647; 20120135039; 20120135503; 20120141493; 20120142079; 20120142080; 20120144509; 20120164687; 20120189657; 20120189661; 20120208866; 20120225454; 20120237491; 20120237537; 20120237544; 20120258128; 20120258129; 20120258135; 20120276167; 20120282181; 20120282291; 20120288523; 20120294948; 20120301422; 20120315278; 20130004547; 20130018089; 20130040370; 20130058997; 20130064845; 20130078275; 20130078278; 20130084307; 20130095131; 20130096103; 20130101523; 2010110249; 20130121968; 20130149321; 20130156809; 20130177589; 20130177593; 20130217063; 20130236948; 20130251719; 20130266635; 20130267481; 20130273144; 20130302380; 20130315950; 20130330824; 20130336990; 20130337012; 20130337545; 20140004178; 20140004193; 20140010844; 20140017279; 20140017285; 20140037691; 20140056940; 20140065187; 20140093477; 20140093954; 20140099320; 20140112951; 20140134662; 20140155343; 20140178425; 20140186398; 20140186401; 20140187612; 20140193459; 20140206064; 20140212396; 20140220661; 20140234310; 20140234379; 20140271563; 20140271719; 20140294883; 20140322249; 20140322265; 20140322267; 20140322268; 20140335125; 20140341921; 20140341942; 20140341970; 20140341974; 20140356415; 20140369986; 20140370036; 20140370057; 20140371428; 20150017138; 20150017191; 20150017204; 20150030573; 20150037370; 20150050311; 20150056246; 20150071994; 20150093824; 20150125485; 20150125921; 20150132335; 20150140028; 20150140034; 20150140037; 20150165011; 20150174178; 20150182611; 20150184167; 20150190500; 20150196659; 20150202276; 20150204845; 20150218254; 20150219645; 20150225692; 20150238589; 20150258190; 20150265696; 20150273045; 20150316567; 20150321037; 20150335736; 20150343050; 20150376242; 20160000896; 20160022592; 20160030494; 20160045591; 20160054299; 20160058860; 20160074505; 20160090395; 20160101168; 20160103127; 20160108096; 20160136285; 20160136294; 20160158334; 20160158335; 20160169921; 20160175415; 20160175428; 20160193256; 20160193257; 20160199422; 20160199474; 20160206727; 20160208261; 20160213770; 20160220652; 20160222393; 20160228523; 20160228530; 20160243204; 20160250311; 20160263209; 20160289287; 20160317637; 20160324783; 20160324939; 20160346381; 20160354462; 20160366862; 20160367650; 20160369282; 20170007683; 20170014513; 20170015735; 20170021011; 20170028048; 20170042987; 20170042996; 20170051260; 20170072042; 20170080078; 20170081642; 20170081671; 20170095548; 20170106028; 20170106074; 20170114319; 20170129942; 20170136102; 20170136111; 20170143815; 20170145061; 20170145065; 20170151321; 20170157232; 20170157239; 201701740174746; 20170182155; 20170191058; 20170209502; 20170216378; 20170240615; 20170246281; 20170258885; 20170290889; 20170290901; 20170304434; 20170318817; 20170327830; 20170340720; 20170350890; 20170360540; 20170368156; 20170368166; 20180008701; 20180021424; 20180028642; 20180028649; 20180043021; 20180044406; 20180049413; 20180050099; 20180066041; 20180066225; 20180071377; 20180087060; 20180099999; 20180104328; 20180140665; 20180147278; 20180164221; 20180168488; 20180168489; 20180168490; 20180169222; 20180169226; 20180185469; 20180193003; 20180193441; 20180206726; 20180206769; 20180221286; 20180221470; 20180236063; 20180243347; and 20180243348.
Aggarwal et al, J. Exp. Med., 172:1083-1090 (1990).
Amdekar, Sarika, Deepak Dwivedi, Purabi Roy. Sapna Kushwah, and Vinod Singh. “Probiotics: multifarious oral vaccine against infectious traumas.” FEMS Immunology & Medical Microbiology 58, no. 3 (2010): 299-306.
Ananthakrishnan, A. N. Clostridium difficile infection: epidemiology, risk factors and management. Nature Reviews Gastroenterology and Hepatology 8, 17-26, doi:10.1038/nrgastro.2010.190 (2010).
Anerson et al., Environmentally controlled invasion of cancer cells by engineered bacteria, J. Mol. Biol. 355: 619-627 (2006).
Arnold, Heinz, Dirk Bumann, Melanie Felies, Britta Gewecke, Meike Sörensen, J. Engelbert Gessner, Joachim Freihorst, Bernd Ulrich Von Specht, and Ulrich Baumann. “Enhanced immunogenicity in the murine airway mucosa with an attenuated Salmonella live vaccine expressing OprF-OprI from Pseudomonas aeruginosa.” Infection and immunity 72, no. 11 (2004): 6546-6553.
Aslam, Saima, Richard J. Hamill, and Daniel M. Musher. “Treatment of Clostridium difficile-associated disease: old therapies and new strategies.” The Lancet infectious diseases 5, no. 9 (2005): 549-557.
Avezov, E. et al. Lifetime imaging of a fluorescent protein sensor reveals surprising stability of ER thiol redox. The Journal of cell biology 201, 337-349, doi:10.1083/jcb.201211155 (2013).
Baliban, S. M. et al. An optimized, synthetic DNA vaccine encoding the toxin A and toxin B receptor binding domains of Clostridium difficile induces protective antibody responses in vivo. Infect. Immun. 82, 4080-4091, doi:10.1128/IAI.01950-14 (2014).
Baud, David, Françoise Ponci, Martine Bobst, Pierre De Grandi, and Denise Nardelli-Haefliger. “Improved efficiency of a Salmonella-based vaccine against human papillomavirus type 16 virus-like particles achieved by using a codon-optimized version of L1.” Journal of virology 78, no. 23 (2004): 12901-12909.
Bermúdez-Humarán, Luis G. “Lactococcus lactis as a live vector for mucosal delivery of therapeutic proteins.” Human vaccines 5, no. 4 (2009): 264-267.
Best, E. L., Freeman, J. & Wilcox, M. H. Models for the study of Clostridium difficile infection. Gut microbes 3, 145-167, doi:10.4161/gmic.19526 (2012).
Bézay, N. et al. Safety, immunogenicity and dose response of VLA84, a new vaccine candidate against Clostridium difficile, in healthy volunteers. Vaccine, doi:10.1016/j.vaccine.2016.03.098 (2016).
Black et al J. Infect. Dis., 155:1260-1265 (1987).
Blisnick, Thierry, Patrick Ave, Michel Huerre, Elisabeth Carniel, and Christian E. Demeure. “Oral vaccination against bubonic plague using a live avirulent Yersinia pseudotuberculosis strain.” Infection and immunity 76, no. 8 (2008): 3808-3816.
Bolhassani, Azam, and Farnaz Zahedifard. “Therapeutic live vaccines as a potential anticancer strategy.” International journal of cancer 131, no. 8 (2012): 1733-1743.
Bonifacino, Juan, Mary Dasso, Joe Harford, Jennifer Lippincott-Schwartz, and Kenneth Yamada. Current Protocols In Cell Biology. 2001.
Branger, Christine G., Roy Curtiss III, Robert D. Perry, and Jacqueline D. Fetherston. “Oral vaccination with different antigens from Yersinia pestis KIM delivered by live attenuated Salmonella typhimurium elicits a protective immune response against plague.” In The Genus Yersinia, pp. 387-399. Springer, New York, N.Y., 2007.
Brett et al, Immunol., 80:306-312 (1993).
Brown, J. Infect. Dis., 155:86-92 (1987).
Bruhn, Kevin W., Noah Craft, and Jeff F. Miller. “Listeria as a vaccine vector.” Microbes and infection 9, no. 10 (2007): 1226-1235.
Bruxelle, Jean-François, Séverine Péchiné, and Anne Collignon. “Immunization Strategies Against Clostridium difficile.” In Updates on Clostridium difficile in Europe, pp. 197-225. Springer, Cham, 2018. doi:10.1007/978-3-319-72799-8_12.
Buckley, Anthony M., Jinhong Wang, Debra L. Hudson, Andrew J. Grant, Michael A.

Jones, Duncan J. Maskell, and Mark P. Stevens. “Evaluation of live-attenuated Salmonella vaccines expressing Campylobacter antigens for control of C. jejuni in poultry.” Vaccine 28, no. 4 (2010): 1094-1105.
Bumann, Dirk. “In vivo visualization of bacterial colonization, antigen expression, and specific T-cell induction following oral administration of live recombinant Salmonella enterica serovar Typhimurium.” Infection and immunity 69, no. 7 (2001): 4618-4626.
Cardenas et al, Vacc., 11:126-135 (1993).
Cardenas, Lucia, and J. D. Clements. “Oral immunization using live attenuated Salmonella spp. as carriers of foreign antigens.” Clinical microbiology reviews 5, no. 3 (1992): 328-342.
Carter, G. P. et al. Defining the Roles of TcdA and TcdB in Localized Gastrointestinal Disease, Systemic Organ Damage, and the Host Response during Clostridium difficile Infections. mBio 6, doi:10.1128/mBio.00551-15 (2015).
Carter, G. P., Rood, J. I. & Lyras, D. The role of toxin A and toxin B in Clostridium difficile-associated disease: Past and present perspectives. Gut microbes 1, 58-64, doi:10.4161/gmic.1.1.10768 (2010).
Chabalgoity, José A., Gordon Dougan, Pietro Mastroeni, and Richard J. Aspinall. “Live bacteria as the basis for immunotherapies against cancer.” Expert review of vaccines 1, no. 4 (2002): 495-505.
Charbit et al, Vacc., 11:1221-1228 (1993).
Chatfield et al, Bio/Technology, 10:888-892 (1992)).
Chatfield et al, Vaccine, 10:8-11 (1992).
Cheminay, Cédric, Annette Möhlenbrink, and Michael Hensel. “Intracellular Salmonella inhibit antigen presentation by dendritic cells.” The Journal of Immunology 174, no. 5 (2005): 2892-2899.
Chen G, Dai Y, Chen J, Wang X, Tang B, Zhu Y, et al. Oral delivery of the Sj23LHD-GST antigen by Salmonella typhimurium type III secretion system protects against Schistosoma japonicum infection in mice. PLoS neglected tropical diseases. 2011; 5(9):e1313.
Chen, Inês, Theresa M. Finn, Liu Yanqing, Qi Guoming, Rino Rappuoli, and Mariagrazia Pizza. “A Recombinant Live Attenuated Strain of Vibrio cholerae Induces Immunity against Tetanus Toxin and Bordetella pertussis Tracheal Colonization Factor.” Infection and immunity 66, no. 4 (1998): 1648-1653.
Chen, X. et al. A Mouse Model of Clostridium difficile-Associated Disease. Gastroenterology 135, 1984-1992, doi:10.1053/j.gastro.2008.09.002 (2008).
Clairmont C, Lee K C, Pike J, Ittensohn M, Low K B, Pawelek J, Bermudes D, Brecher S M, Margitich D, Turnier J, Li Z, Luo X, King I, Zheng L M. 2000. Biodistribution and genetic stability of the novel antitumor agent VNP20009, a genetically modified strain of Salmonella typhimurium. The Journal of infectious diseases 181:1996-2002.
Clark-Curtiss, J. E. & Curtiss, R. Salmonella Vaccines: Conduits for Protective Antigens. Journal of immunology (Baltimore, Md.: 1950) 200, 39-48, doi:10.4049/jimmunol.1600608 (2018).
Cohen, O. R., Steele, J. A., Zhang, Q., Schmidt, D. J. & one, W.-Y. Systemically administered IgG anti-toxin antibodies protect the colonic mucosa during infection with Clostridium difficile in the piglet model. PLoS One, doi:10.1371/journal.pone.0111075 (2014).
Coligan et al., eds., Current Protocols in Protein Sciences 2009, Wiley Intersciences.
Cote-Sierra, Javier, Erik Jongert, Amin Bredan, Dinesh C. Gautam, M. Parkhouse, Pierre Cornelis, Patrick De Baetselier, and Hilde Revets. “A new membrane-bound OprI lipoprotein expression vector: high production of heterologous fusion proteins in Gram (−) bacteria and the implications for oral vaccination.” Gene 221, no. 1 (1998): 25-34.
Cronan M R, Matty M A, Rosenberg A F, Blanc L, Pyle C J, Espenschied S T, et al. An explant technique for high-resolution imaging and manipulation of mycobacterial granulomas. Nature Methods. 2018; 15(12):1098-107.
Cuadro et al., Infect. Immun. 72: 2810-2816 (2004).
Cuburu N, Kim R, Guittard G C, Thompson C D, Day P M, Hamm D E, et al. A Prime-Pull-Amplify Vaccination Strategy To Maximize Induction of Circulating and Genital-Resident Intraepithelial CD8(+) Memory T Cells. Journal of immunology (Baltimore, Md.: 1950). 2019; 202(4):1250-64.
Curtiss et al, Dev. Biol. Stand., 82:23-33 (1994).
Curtiss, In: New Generation Vaccines: The Molecular Approach, Ed., Marcel Dekker, Inc., New York, N.Y., pages 161-188 and 269-288 (1989).
Darji, Ayub, Carlos A. Guzmin, Birgit Gerstel, Petra Wachholz, Kenneth N. Timmis, Jürgen Wehland, Trinad Chakraborty, and Siegfried Weiss. “Oral somatic transgene vaccination using attenuated S. typhimurium.” Cell 91, no. 6 (1997): 765-775.
Davis et al., Basic Methods in Molecular Biology, Elsevier Science Publishing, Inc., New York, USA (1995).
Del Rio, Beatriz, Raymond J. Dattwyler, Miguel Aroso, Vera Neves, Luciana Meirelles, Jos F M L Seegers, and Maria Gomes-Solecki. “Oral immunization with recombinant Lactobacillus plantarum induces a protective immune response in mice with Lyme disease.” Clinical and Vaccine Immunology 15, no. 9 (2008): 1429-1435.
Detmer, Ann, and Jacob Glenting. “Live bacterial vaccines-a review and identification of potential hazards.” Microbial cell factories 5, no. 1 (2006): 23.
Doggett et al, Infect. Immun., 61:1859-1866 (1993).
Donald, R. G. K. et al. A novel approach to generate a recombinant toxoid vaccine against Clostridium difficile. Microbiology 159, 1254-1266, doi:10.1099/mic.0.066712-0 (2013).
Dougan, G., C. E. Hormaeche, and D. J. Maskell. “Live oral Salmonella vaccines: potential use of attenuated strains as carriers of heterologous antigens to the immune system.” Parasite immunology 9, no. 2 (1987): 151-160.
Du, Aifang, and Suhua Wang. “Efficacy of a DNA vaccine delivered in attenuated Salmonella typhimurium against Eimeria tenella infection in chickens.” International Journal for Parasitology 35, no. 7 (2005): 777-785.
Ebenezer J A, Christensen J M, Oliver B G, Oliver R A, Tjin G, Ho J, et al. Periostin as a marker of mucosal remodelling in chronic rhinosinusitis. Rhinology. 2017; 55(3):234-41.
Fayolle, C., D. O'Callaghan, P. Martineau, A. Charbit, J. M. Clément, M. Hofnung, and C. Leclerc. “Genetic control of antibody responses induced against an antigen delivered by recombinant attenuated Salmonella typhimurium.” Infection and immunity 62, no. 10 (1994): 4310-4319.
Flynn et al, Mol. Microbiol., 4:2111-2118 (1990).
Flynn, Cell. Molec. Biol., 40(Suppl. 1):31-36 (1994).
Formal et al, Infect. Immun., 34:746-751 (1981).
Fraillery, Dominique, David Baud, Susana Yuk-Ying Pang, John Schiller, Martine Bobst, Nathalie Zosso, Françoise Ponci, and Denise Nardelli-Haefliger. “Salmonella enterica serovar Typhi Ty21a expressing human papillomavirus type 16 L1 as a potential live vaccine against cervical cancer and typhoid fever.” Clin. Vaccine Immunol. 14, no. 10 (2007): 1285-1295.
Freer G, Pistello M. 2018. Varicella-zoster virus infection: natural history, clinical manifestations, immunity and current and future vaccination strategies. New Microbiol 41:95-105.
Freshney, R. Ian, Culture of Animal Cells: A Manual of Basic Technique (Wiley-Liss) 5th edition (2005).
Frey A, Di Canzio J, Zurakowski D. A statistically defined endpoint titer determination method for immunoassays. Journal of immunological methods. 1998; 221(1-2):35-41.
Gahan, Michelle E., Diane E. Webster, Steven L. Wesselingh, and Richard A. Strugnell. “Impact of plasmid stability on oral DNA delivery by Salmonella enterica serovar Typhimurium.” Vaccine 25, no. 8 (2007): 1476-1483.
Galen, J. E., Buskirk, A. D., Tennant, S. M. & Pasetti, M. F. Live Attenuated Human Salmonella Vaccine Candidates: Tracking the Pathogen in Natural Infection and Stimulation of Host Immunity. EcoSal Plus 7, doi:10.1128/ecosalplus.ESP-0010-2016 (2016).
Galen, James E., Oscar G. Gomez-Duarte, Genevieve A. Losonsky, Jane L. Halpern, Carol S. Lauderbaugh, Shevon Kaintuck, Mardi K. Reymann, and Myron M. Levine. “A murine model of intranasal immunization to assess the immunogenicity of attenuated Salmonella typhi live vector vaccines in stimulating serum antibody responses to expressed foreign antigens.” Vaccine 15, no. 6-7 (1997): 700-708.
Galen et al., Infection and Immunity, 72: 7096-7106 (2004).
Gao et al, Infect. Immun., 60:3780-3789 (1992).
Garmory, Helen S., Sophie E C Leary, Kate F. Griffin, E. Diane Williamson, Katherine A. Brown, and Richard W. Titball. “The use of live attenuated bacteria as a delivery system for heterologous antigens.” Journal of drug targeting 11, no. 8-10 (2003): 471-479.
Gentschev I, Spreng S, Sieber H, Ures J, Mollet F, Collioud A, et al. Vivotif—a ‘magic shield’ for protection against typhoid fever and delivery of heterologous antigens. Chemotherapy. 2007; 53(3):177-80.
Georgiou, George, Christos Stathopoulos, Patrick S. Daugherty, Amiya R. Nayak, Brent L. Iverson, and Roy Curtiss III. “Display of heterologous proteins on the surface of microorganisms: from the screening of combinatorial libraries to live recombinant vaccines.” Nature biotechnology 15, no. 1 (1997): 29-34.
Gerding, Dale N. “Clostridium difficile infection prevention: biotherapeutics, immunologics, and vaccines.” Discovery medicine 13, no. 68 (2012): 75-83.
Gerlach R G, Hensel M. Salmonella pathogenicity islands in host specificity, host pathogen-interactions and antibiotics resistance of Salmonella enterica. Berliner und Munchener tierarztliche Wochenschrift. 2007; 120(7-8):317-27.
Ghose, C. et al. Toll-like receptor 5-dependent immunogenicity and protective efficacy of a recombinant fusion protein vaccine containing the nontoxic domains of Clostridium difficile toxins A and B and Salmonella enterica serovar typhimurium flagellin in a mouse model of Clostridium difficile disease. Infect. Immun. 81, 2190-2196, doi:10.1128/IAI.01074-12 (2013).
Giannasca, Paul J., and Michel Warny. “Active and passive immunization against Clostridium difficile diarrhea and colitis.” Vaccine 22, no. 7 (2004): 848-856.
Glenting J, Wessels S. Ensuring safety of DNA vaccines. Microbial cell factories. 2005; 4:26.
Gonzalez et al, J. Infect. Dis., 169:927-931 (1994).
Gradoni, L. “An update on antileishmanial vaccine candidates and prospects for a canine Leishmania vaccine.” Veterinary parasitology 100, no. 1-2 (2001): 87-103.
Grangette, Corinne, Heide Müller-Alouf, Denise Goudercourt, Marie-Claude Geoffroy, Mireille Turneer, and Annick Mercenier. “Mucosal immune responses and protection against tetanus toxin after intranasal immunization with recombinant Lactobacillus plantarum.” Infection and immunity 69, no. 3 (2001): 1547-1553.
Grangette, Corinne, Heide Müller-Alouf, Marie-Claude Geoffroy, Denise Goudercourt, Mireille Turneer, and Annick Mercenier. “Protection against tetanus toxin after intragastric administration of two recombinant lactic acid bacteria: impact of strain viability and in vivo persistence.” Vaccine 20, no. 27-28 (2002): 3304-3309.
Greenberg, R. N., Marbury, T. C., Foglia, G. & Warny, M. Phase I dose finding studies of an adjuvanted Clostridium difficile toxoid vaccine. Vaccine 30, 2245-2249, doi:10.1016/j.vaccine.2012.01.065 (2012).
Grice et al., Topographical and temporal diversity of the human skin microbiome, Science 324: 1190-1192; A framework for human microbiome research; The Human Microbiome Project Consortium, 14 Jun., 2012 Nature 486, 215-221.
Guimaraes et al., Use of Native Lactococci as Vehicles for Delivery of DNA into Mammalian Epithelial Cells, Appl Environ Microbiol. 2006 November; 72(11): 7091-7097.
Guo, Shanguang, Weiwei Yan, Sean P. McDonough, Nengfeng Lin, Katherine J. Wu, Hongxuan He, Hua Xiang, Maosheng Yang, Maira Aparecida S. Moreira, and Yung-Fu Chang. “The recombinant Lactococcus lactis oral vaccine induces protection against C. difficile spore challenge in a mouse model.” Vaccine 33, no. 13 (2015): 1586-1595, doi:10.1016/j.vaccine.2015.02.006.
Gupta and Lee, 2008 Biotechnology and Bioengineering, 101: 967-974.
Guzman, Carlos A., Stefan Borsutzky, Monika Griot-Wenk, Ian C. Metcalfe, Jon Pearman, Andre Collioud, Didier Favre, and Guido Dietrich. “Vaccines against typhoid fever.” Vaccine 24, no. 18 (2006): 3804-3811.
Hahn, Heinz P., and Bernd-Ulrich von Specht. “Secretory delivery of recombinant proteins in attenuated Salmonella strains: potential and limitations of Type I protein transporters.” FEMS Immunology & Medical Microbiology 37, no. 2-3 (2003): 87-98.
Hansson, Marianne, and Stefan Sta. “Design and production of recombinant subunit vaccines.” Biotechnology and applied biochemistry 32, no. 2 (2000): 95-107.
Haraga A, Ohlson M B, Miller S I. Salmonellae interplay with host cells. Nature reviews Microbiology. 2008; 6(1):53-66.
Harrison, J. A., B. Villarreal-Ramos, P. Mastroeni, R. Demarco de Hormaeche, and C. E. Hormaeche. “Correlates of protection induced by live Aro—Salmonella typhimurium vaccines in the murine typhoid model.” Immunology 90, no. 4 (1997): 618-625.
Haselbeck, A. H. et al. Current perspectives on invasive nontyphoidal Salmonella disease. Curr. Opin. Infect. Dis. 30, 498-503, doi:10.1097/QCO.0000000000000398 (2017).
Hayashi F, Smith K D, Ozinsky A, Hawn T R, Yi E C, Goodlett D R, et al. The innate immune response to bacterial flagellin is mediated by Toll-like receptor 5. Nature. 2001; 410(6832):1099-1103. doi:10.1038/35074106 (2001).
Heimann, S. M., Cruz Aguilar, M. R., Mellinghof, S. & Vehreschild, M. J. G. T. J. Economic burden and cost-effective management of Clostridium difficile infections. Med. Mal. Infect. 48, 23-29, doi:10.1016/j.medmal.2017.10.010 (2018).
Henderson et al., 2004, Type V secretion pathway: the autotransporter story, Microbiology and Molecular Biology Reviews 68: 692-744.
Hilleman, Dev. Biol. Stand., 82:3-20 (1994); Formal et al, Infect. Immun. 34:746-751 (1981).
Hindle Z, Chatfield S N, Phillimore J, Bentley M, Johnson J, Cosgrove C A, et al. Characterization of Salmonella enterica derivatives harboring defined aroC and Salmonella pathogenicity island 2 type III secretion system (ssaV) mutations by immunization of healthy volunteers. Infection and immunity. 2002; 70(7):3457-67.
Hohmann E L, Oletta C A, Loomis W P, Miller S I. Macrophage-inducible expression of a model antigen in Salmonella typhimurium enhances immunogenicity. Proceedings of the National Academy of Sciences of the United States of America. 1995; 92(7):2904-8.
Hone et al, J. Clin. Invest., 90:412-420 (1992).
Hone et al, Microbial. Path., 5:407-418 (1988).
Hone et al, Vaccine, 9:810-816 (1991).
Hong, Huynh A., Krisztina Hitri, Siamand Hosseini, Natalia Kotowicz, Donna Bryan, Fatme Mawas, Anthony J. Wilkinson, Annie van Broekhoven, Jonathan Kearsey, and Simon M. Cutting. “Mucosal antibodies to the C-terminus of toxin A prevent colonization of Clostridium difficile.” Infection and immunity (2017): IAI-01060. doi:10.1128/IAI.01060-16.
Huang, Jen-Min, Michela Sali, Matthew W. Leckenby, David S. Radford, Hong A. Huynh, Giovanni Delogu, Rocky M. Cranenburgh, and Simon M. Cutting. “Oral delivery of a DNA vaccine against tuberculosis using operator-repressor titration in a Salmonella enterica vector.” Vaccine 28, no. 47 (2010): 7523-7528.
Husseiny, Mohamed I., and Michael Hensel. “Evaluation of an intracellular-activated promoter for the generation of live Salmonella recombinant vaccines.” Vaccine 23, no. 20 (2005): 2580-2590.
Ibarra, A. J. & Mortimer, O. Salmonella—the ultimate insider. Salmonella virulence factors that modulate intracellular survival. Cell. Microbiol. 11, 1579-1586, doi:10.1111/j.1462-5822.2009.01368.x (2009).
Innocentin et al., Lactococcus lactis Expressing either Staphylococcus aureus Fibronectin-Binding Protein A or Listeria monocytogenes Internalin A Can Efficiently Internalize and Deliver DNA in Human Epithelial Cells Appl Environ Microbiol. 2009 July; 75(14): 4870-4878).
Izore et al. Structure 2011 19:603-612; Korotkov et al. Nature Reviews Microbiology 2012 10:336-351.
Jabbar, Ibtissam A., Germain J P Fernando, Nick Saunders, Anne Aldovini, Richard Young, Karen Malcolm, and Ian H. Frazer. “Immune responses induced by BCG recombinant for human papillomavirus L1 and E7 proteins.” Vaccine 18, no. 22 (2000): 2444-2453.
Jensen, Eric R., Hao Shen, Felix O. Wettstein, Rafi Ahmed, and Jeff F. Miller. “Recombinant Listeria monocytogenes as a live vaccine vehicle and a probe for studying cell-mediated immunity.” Immunological reviews 158, no. 1 (1997): 147-157.
Jepson M A, Clark M A. The role of M cells in Salmonella infection. Microbes and infection. 2001; 3(14-15):1183-90.
Jose J, Zangen D (2005) Autodisplay of the protease inhibitor aprotinin in Escherichia coli. Biochem Biophys Res Commun 333:1218-1226
Jose, 2006 Applied Microbiol. Biotechnol. 69: 607-614.
Jung et al., 1998, Surface display of Zymomonas mobilis levansucrase by using ice-nucleation protein of Pseudomonas syringae, Nature Biotechnology 16: 576-580.
Kang, Ho Young, Jay Srinivasan, and Roy Curtiss. “Immune responses to recombinant pneumococcal PspA antigen delivered by live attenuated Salmonella enterica serovar Typhimurium vaccine.” Infection and immunity 70, no. 4 (2002): 1739-1749.
Kardani, K., Bolhassani, A. & Vaccine, S.-S. Prime-boost vaccine strategy against viral infections: Mechanisms and benefits. Vaccine (2016).
Karsten, Verena, Sean R. Murray, Jeremy Pike, Kimberly Troy, Martina Ittensohn, Manvel Kondradzhyan, K. Brooks Low, and David Bermudes. “msbB deletion confers acute sensitivity to CO 2 in Salmonella enterica serovar Typhimurium that can be suppressed by a loss-of-function mutation in zwf.” BMC microbiology 9, no. 1 (2009): 170.
Kelly, Ciaran P., and Lorraine Kyne. “The host immune response to Clostridium difficile.” Journal of medical microbiology 60, no. 8 (2011): 1070-1079.
Kendrew et al. (eds.), Molecular Biology and Biotechnology: a Comprehensive Desk Reference, published by VCH Publishers, Inc., 1995 (ISBN 1-56081-569-8).
Killeen, K., D. Spriggs, and J. Mekalanos. “Bacterial mucosal vaccines: Vibrio cholerae as a live attenuated vaccine/vector paradigm.” In Defense of Mucosal Surfaces: Pathogenesis, Immunity and Vaccines, pp. 237-254. Springer, Berlin, Heidelberg, 1999.
Kim et al., 2000, Bacterial surface display of an enzyme library for selective screening of improved cellulase variants, Applied and Environmental Microbiology 66: 788-793.
Kim, K. S., M. C. Jenkins, and HYUN S. Lillehoj. “Immunization of chickens with live Escherichia coli expressing Eimeria acervulina merozoite recombinant antigen induces partial protection against coccidiosis.” Infection and immunity 57, no. 8 (1989): 2434-2440.
Klauser et al., 1990 EMBO Journal 9: 1991-1999
Knudsen, Maria Lisa, Karl Ljungberg, Maria Kakoulidou, Linda Kostic, David Hallengaird, Juan Garcia-Arriaza, Andres Merits, Mariano Esteban, and Peter Liljestrim. “Kinetic and phenotypic analysis of CD8+ T cell responses after priming with alphavirus replicons and homologous or heterologous booster immunizations.” Journal of virology (2014): JVI-02223. doi:10.1128/JVI.02223-14.
Kociolek, L. K. & Gerding, D. N. Breakthroughs in the treatment and prevention of Clostridium difficile infection. Nature reviews. Gastroenterology & hepatology, doi:10.1038/nrgastro.2015.220 (2016).
Kotton, Camille N., and Elizabeth L. Hohmann. “Enteric pathogens as vaccine vectors for foreign antigen delivery.” Infection and immunity 72, no. 10 (2004): 5535-5547.
Kotzsch et al. 2011, A secretory system for bacterial production of high-profile protein targets, Protein Science 20: 597-609
Kuehne, S. A., Cartman, S. T., Heap, J. T. & Nature, K.-M. L. The role of toxin A and toxin B in Clostridium difficile infection. Nature (2010).
Kühnel et al., 2004 PNAS 101: 17027-17032
Kuipers, Kirsten, Maria H. Daleke-Schermerhorn, Wouter S P Jong, M. Corinne, Fred van Opzeeland, Elles Simonetti, Joen Luirink, and Marien I. de Jonge. “Salmonella outer membrane vesicles displaying high densities of pneumococcal antigen at the surface offer protection against colonization.” Vaccine 33, no. 17 (2015): 2022-2029.
Lagranderie et al, Vaccine, 11:1283-1290 (1993).
Lakhashe, S. K., Byrareddy, S. N., Zhou, M. & Vaccine, B.-B. C. Multimodality vaccination against clade C SHIV: partial protection against mucosal challenges with a heterologous tier 2 virus. Vaccine (2014).
Lee A K, Detweiler C S, Falkow S. OmpR regulates the two-component system SsrA-ssrB in Salmonella pathogenicity island 2. Journal of bacteriology. 2000; 182(3):771-81.
Lee, Bruce Y., Michael J. Popovich, Ye Tian, Rachel R. Bailey, Paul J. Ufberg, Ann E. Wiringa, and Robert R. Muder. “The potential value of Clostridium difficile vaccine: an economic computer simulation model.” Vaccine 28, no. 32 (2010): 5245-5253.
Lee, Jong-Soo, Kwang-Soon Shin, Jae-Gu Pan, and Chul-Joong Kim. “Surface-displayed viral antigens on Salmonella carrier vaccine.” Nature biotechnology 18, no. 6 (2000): 645.
Levine et al, In: Vibrio cholerae, Molecular to Global Perspectives, Wachsmuth et al, Eds, ASM Press, Washington, D.C., pages 395-414 (1994).
Levine et al, J. Clin. Invest., 79:888-902 (1987).
Lewin, Benjamin, Genes X, published by Jones & Bartlett Publishing, 2009 (ISBN-10: 0763766321).
Li, Long, Weihuan Fang, Jianrong Li, Li Fang, Yaowei Huang, and Lian Yu. “Oral DNA vaccination with the polyprotein gene of infectious bursal disease virus (IBDV) delivered by attenuated Salmonella elicits protective immune responses in chickens.” Vaccine 24, no. 33-34 (2006): 5919-5927.
Liljeqvist, Sissela, and Stefan Stahl. “Production of recombinant subunit vaccines: protein immunogens, live delivery systems and nucleic acid vaccines.” Journal of biotechnology 73, no. 1 (1999): 1-33.
Loessner, Holger, and Siegfried Weiss. “Bacteria-mediated DNA transfer in gene therapy and vaccination.” Expert opinion on biological therapy 4, no. 2 (2004): 157-168.
Loessner, Holger, Anne Endmann, Sara Leschner, Heike Bauer, Andrea Zelmer, Susanne zur Lage, Kathrin Westphal, and Siegfried Weiss. “Improving live attenuated bacterial carriers for vaccination and therapy.” International Journal of Medical Microbiology 298, no. 1-2 (2008): 21-26.
Lossner et al., Cell Microbiol. 9: 1529-1537 (2007).
Low et al., 1999, Lipid A mutant salmonella with suppressed virulence and TNFα induction retain tumor-targeting in vivo, Nature Biotechnol. 17: 37-41.

Luke, C. J. & review of vaccines, S.-K. Improving pandemic H5N1 influenza vaccines by combining different vaccine platforms. Expert review of vaccines, doi:10.1586/14760584.2014.922416 (2014).
Makvandi, Manoochehr, Ali Teimoori, Mehdi Parsa Nahad, Ali Khodadadi, and Milad Zandi. “Expression of Salmonella typhimurium and Escherichia coli flagellin protein and its functional characterization as an adjuvant.” Microbial pathogenesis 118 (2018): 87-90.
Mastroeni, Pietro, Bernardo Villarreal-Ramos, and Carlos E. Hormaeche. “Role of T cells, TNFα and IFNγ in recall of immunity to oral challenge with virulent salmonellae in mice vaccinated with live attenuated aro—salmonella vaccines.” Microbial pathogenesis 13, no. 6 (1992): 477-491.
Mather, Jennie P. and Barnes, David, eds., Animal Cell Culture Methods (Methods in Cell Biology, Vol. 57, Academic Press, 1st edition, 1998.
McSorley, Stephen J., Damo Xu, and FYs Liew. “Vaccine efficacy of Salmonella strains expressing glycoprotein 63 with different promoters.” Infection and immunity 65, no. 1 (1997): 171-178.
Medina, Eva, and Carlos Alberto Guzmán. “Use of live bacterial vaccine vectors for antigen delivery: potential and limitations.” Vaccine 19, no. 13-14 (2001): 1573-1580.
Merck Research Laboratories, “The Merck Manual of Diagnosis and Therapy”, 19th Edition, 2006 (ISBN 0-911910-19-0).
Metzger, Wolfram G., E. Mansouri, M. Kronawitter, Susanne Diescher, Meike Soerensen, Robert Hurwitz, Dirk Bumann, Toni Aebischer, B-U. Von Specht, and Thomas F. Meyer. “Impact of vector-priming on the immunogenicity of a live recombinant Salmonella enterica serovar typhi Ty21a vaccine expressing urease A and B from Helicobacter pylori in human volunteers.” Vaccine 22, no. 17-18 (2004): 2273-2277.
Mielcarek, Nathalie, Sylvie Alonso, and Camille Locht. “Nasal vaccination using live bacterial vectors.” Advanced drug delivery reviews 51, no. 1-3 (2001): 55-69.
Miller S I, Pulkkinen W S, Selsted M E, Mekalanos J J. Characterization of defensin resistance phenotypes associated with mutations in the phoP virulence regulon of Salmonella typhimurium. Infection and immunity. 1990; 58(11):3706-10.
Mims et al, In: Medical Microbiology, Eds., Mosby-Year Book Europe Ltd., London (1993)).
Mims, The Pathogenesis of Infectious Disease, Academic Press, London (1987).
Mohamadzadeh et al., PNAS Mar. 17, 2009 vol. 106 no. 11 4331-4336.
Mohamadzadeh, Mansour, Tri Duong, Timothy Hoover, and Todd R. Klaenhammer. “Targeting mucosal dendritic cells with microbial antigens from probiotic lactic acid bacteria.” Expert review of vaccines 7, no. 2 (2008): 163-174.
Murray et al., Hot spot for a large deletion in the 18-19 Cs region confers a multiple phenotype in Salmonella enterica serovar Typhimurium strain ATCC 14028. Journal of Bacteriology 186: 8516-8523 (2004).
Nagarajan, Arvindhan G., Sudhagar V. Balasundaram, Jessin Janice, Guruswamy Karnam, Sandeepa M. Eswarappa, and Dipshikha Chakravortty. “SopB of Salmonella enterica serovar Typhimurium is a potential DNA vaccine candidate in conjugation with live attenuated bacteria.” Vaccine 27, no. 21 (2009): 2804-2811.
Nemunaitis et al., 2003, Pilot trial of genetically modified, attenuated Salmonella expressing the E. coli cytosine deaminase gene in refractory cancer patients, Cancer Gene Therapy 10: 737-744
Newton et al., 1995, Res. Microbiol. 146: 203-216
Niedergang, Florence, Jean-Claude Sirard, Corinne Tallichet Blanc, and Jean-Pierre Kraehenbuhl. “Entry and survival of Salmonella typhimurium in dendritic cells and presentation of recombinant antigens do not require macrophage-specific virulence factors.” Proceedings of the National Academy of Sciences 97, no. 26 (2000): 14650-14655.
Noriega et al, Infect. Immun., 62:5168-5172 (1994).
Oggioni, Marco R., Riccardo Manganelli, Mario Contorni, Massimo Tommasino, and Gianni Pozzi. “Immunization of mice by oral colonization with live recombinant commensal streptococci.” Vaccine 13, no. 8 (1995): 775-779.
Okan, Nihal A., Patricio Mena, Jorge L. Benach, James B. Bliska, and A. Wali Karzai. “The smpB-ssrA mutant of Yersinia pestis functions as a live attenuated vaccine to protect mice against pulmonary plague infection.” Infection and immunity 78, no. 3 (2010): 1284-1293.
Pace, John Lee, Richard Ives Walker, and Steven Michael Frey. “Methods for producing enhanced antigenic campylobacter bacteria and vaccines.” U.S. Pat. No. 5,679,564, issued Oct. 21, 1997.
Paglia, Paola, Ivano Arioli, Nicole Frahm, Trinad Chakraborty, Mario P. Colombo, and Carlos A. Guzman. “The defined attenuated Listeria monocytogenes Δmpl2 mutant is an effective oral vaccine carrier to trigger a long-lasting immune response against a mouse fibrosarcoma.” European journal of immunology 27, no. 6 (1997): 1570-1575.
Panthel, K., Meinel, K. M., Sevil Domenech, V. E. E., Triilzsch, K. & Rüssmann, H. Salmonella type III-mediated heterologous antigen delivery: a versatile oral vaccination strategy to induce cellular immunity against infectious agents and tumors. International journal of medical microbiology: IJMM 298, 99-103, doi:10.1016/j.ijmm.2007.07.002 (2008).
Pasetti, Marcela F., Myron M. Levine, and Marcelo B. Sztein. “Animal models paving the way for clinical trials of attenuated Salmonella enterica serovar Typhi live oral vaccines and live vectors.” Vaccine 21, no. 5-6 (2003): 401-418.
Paterson, Yvonne, Patrick D. Guirnalda, and Laurence M. Wood. “Listeria and Salmonella bacterial vectors of tumor-associated antigens for cancer immunotherapy.” In Seminars in immunology, vol. 22, no. 3, pp. 183-189. Academic Press, 2010.
Paterson, Yvonne. “Specific immunotherapy of cancer using a live recombinant bacterial vaccine vector.” U.S. Pat. No. 6,051,237, issued Apr. 18, 2000.
Patyar, S., R. Joshi, D S Prasad Byrav, A. Prakash, B. Medhi, and B. K. Das. “Bacteria in cancer therapy: a novel experimental strategy.” Journal of biomedical science 17, no. 1 (2010): 21.
Pawelek, John M., K. Brooks Low, and David Bermudes. “Tumor-targeted Salmonella as a novel anticancer vector.” Cancer research 57, no. 20 (1997): 4537-4544.
Péchiné, Séverine, Cécile Denève, Alban Le Monnier, Sandra Hoys, Claire Janoir, and Anne Collignon. “Immunization of hamsters against Clostridium difficile infection using the Cwp84 protease as an antigen.” FEMS Immunology & Medical Microbiology 63, no. 1 (2011): 73-81.
Péchiné, Séverine, Claire Janoir, Hélène Boureau, Aude Gleizes, Nicolas Tsapis, Sandra Hoys, Elias Fattal, and Anne Collignon. “Diminished intestinal colonization by Clostridium difficile and immune response in mice after mucosal immunization with surface proteins of Clostridium difficile.” Vaccine 25, no. 20 (2007): 3946-3954.
Penha Filho, R. A. et al. Humoral and cellular immune response generated by different vaccine programs before and after Salmonella Enteritidis challenge in chickens. Vaccine 30, 7637-7643, doi:10.1016/j.vaccine.2012.10.020 (2012).
Poltorak A, He X, Smirnova I, Liu M Y, Van Huffel C, Du X, et al. Defective LPS signaling in C3H/HeJ and C57BL/10ScCr mice: mutations in Tlr4 gene. Science (New York, N.Y.). 1998; 282(5396):2085-8.
Porter, Robert S., et al. (eds.), The Encyclopedia of Molecular Biology, published by Blackwell Science Ltd., 1994 (ISBN 0-632-02182-9).
Prisco, A. & De Bernardis, P., Memory immune response: a major challenge in vaccination. Biomolecular concepts (2012).
Qu, Daofeng, Suhua Wang, Weiming Cai, and Aifang Du. “Protective effect of a DNA vaccine delivered in attenuated Salmonella typhimurium against Toxoplasma gondii infection in mice.” Vaccine 26, no. 35 (2008): 4541-4548.
Reigadas, E., L. Alcala, M. Marín, A. Martin, C. Iglesias, and E. Bouza. “Role of binary toxin in the outcome of Clostridium difficile infection in a non-027 ribotype setting.” Epidemiology & Infection 144, no. 2 (2016): 268-273. doi:10.1017/s095026881500148x (2015).
Reveneau, Nathalie, Marie-Claude Geoffroy, Camille Locht, Patrice Chagnaud, and Annick Mercenier. “Comparison of the immune responses induced by local immunizations with recombinant Lactobacillus plantarum producing tetanus toxin fragment C in different cellular locations.” Vaccine 20, no. 13-14 (2002): 1769-1777.
Robinson, Karen, Lisa M. Chamberlain, Karin M. Schofield, Jeremy M. Wells, and Richard W F Le Page. “Oral vaccination of mice against tetanus with recombinant Lactococcus lactis.” Nature biotechnology 15, no. 7 (1997): 653.
Rosenkranz, Claudia D., Damasia Chiara, Caroline Agorio, Adriana Baz, Marcela F. Pasetti, Fernanda Schreiber, Silvia Dematteis, Miguel Martinez, Marcelo B. Sztein, and Jose A. Chabalgoity. “Towards new immunotherapies: targeting recombinant cytokines to the immune system using live attenuated Salmonella.” Vaccine 21, no. 7-8 (2003): 798-801.
Ross, Bruce C., Larissa Czajkowski, Dianna Hocking, Mai Margetts, Elizabeth Webb, Linda Rothel, Michelle Patterson et al. “Identification of vaccine candidate antigens from a genomic analysis of Porphyromonas gingivalis.” Vaccine 19, no. 30 (2001): 4135-4142.
Rota P A, Khan A S, Durigon E, Yuran T, Villamarzo Y S, Bellini W J. 1995. Detection of measles virus RNA in urine specimens from vaccine recipients. J Clin Microbiol 33:2485-2488.
Royo et al., Nature Methods 4: 937-942 (2007).
Rupnik, M., Wilcox, M. H. & Microbiology, G. D. N. Clostridium difficile infection: new developments in epidemiology and pathogenesis. Clostridium difficile infection: new developments in epidemiology and pathogenesis, doi:10.1038/nrmicro2164 (2009).
Rutherford and Mourez 2006 Microbial Cell Factories 5: 22
Ryan, Edward T., Joan R. Butterton, Rex Neal Smith, Patricia A. Carroll, Thomas I. Crean, and Stephen B. Calderwood. “Protective immunity against Clostridium difficile toxin A induced by oral immunization with a live, attenuated Vibrio cholerae vector strain.” Infection and immunity 65, no. 7 (1997): 2941-2949.
Sambrook et al., Molecular Cloning: A Laboratory Manual (3 ed.), Cold Spring Harbor Laboratory Press, Cold Spring Harbor, N.Y., USA (2001).
Samuelson et al., Display of proteins on bacteria, J. Biotechnology 96: 129-154 (2002).
Santos, Renato L., Shuping Zhang, Renée M. Tsolis, Robert A. Kingsley, L. Garry Adams, and Andreas J. Baumler. “Animal models of Salmonella infections: enteritis versus typhoid fever.” Microbes and Infection 3, no. 14-15 (2001): 1335-1344.
Sbrogio-Almeida, M. E., Tainá Mosca, L. M. Massis, I. A. Abrahamsohn, and L. C. S. Ferreira. “Host and bacterial factors affecting induction of immune responses to flagellin expressed by attenuated Salmonella vaccine strains.” Infection and immunity 72, no. 5 (2004): 2546-2555.
Schafer et al, J. Immunol., 149:53-59 (1992).
Schodel et al, Infect. Immun., 62:1669-1676 (1994).
Schodel et al, J. Immunol., 145:4317-4321 (1990).
Schorr, Joachim, Bernhard Knapp, Erika Hundt, Hans A. Kiipper, and Egon Amann. “Surface expression of malarial antigens in Salmonella typhimurium: induction of serum antibody response upon oral vaccination of mice.” Vaccine 9, no. 9 (1991): 675-681.
Seegers, Jos F M L. “Lactobacilli as live vaccine delivery vectors: progress and prospects.” Trends in biotechnology 20, no. 12 (2002): 508-515.
Shahabi, Vafa, Paulo C. Maciag, Sandra Rivera, and Anu Wallecha. “Live, attenuated strains of Listeria and Salmonella as vaccine vectors in cancer treatment.” Bioengineered bugs 1, no. 4 (2010): 237-245.
Shams, Homayoun. “Recent developments in veterinary vaccinology.” The veterinary journal 170, no. 3 (2005): 289-299.
Shams, Homayoun, Fernando Poblete, Holger Rüssmann, Jorge E. Galán, and Ruben O. Donis. “Induction of specific CD8+ memory T cells and long lasting protection following immunization with Salmonella typhimurium expressing a lymphocytic choriomeningitis MHC class I-restricted epitope.” Vaccine 20, no. 3-4 (2001): 577-585.
Shen, Hao, Mark K. Slifka, Mehrdad Matloubian, Eric R. Jensen, Rafi Ahmed, and Jeff F. Miller. “Recombinant Listeria monocytogenes as a live vaccine vehicle for the induction of protective anti-viral cell-mediated immunity.” Proceedings of the National Academy of Sciences 92, no. 9 (1995): 3987-3991.
Shukarev, Georgi, Benoit Callendret, Kerstin Luhn, Macaya Douoguih, and EBOVAC1 consortium. “A two-dose heterologous prime-boost vaccine regimen eliciting sustained immune responses to Ebola Zaire could support a preventive strategy for future outbreaks.” Human vaccines & immunotherapeutics 13, no. 2 (2017): 266-270. doi:10.1080/21645515.2017.1264755.
Silin, Dmytro S., Oksana V. Lyubomska, Vichai Jirathitikal, and Aldar S. Bourinbaiar. “Oral vaccination: where we are?.” Expert opinion on drug delivery 4, no. 4 (2007): 323-340.
Silva, Adilson Jose da, Teresa Cristina Zangirolami, Maria Teresa Marques Novo-Mansur, Roberto de Campos Giordano, and Elizabeth Angélica Leme Martins. “Live bacterial vaccine vectors: an overview.” Brazilian Journal of Microbiology 45, no. 4 (2014): 1117-1129.
Simonet et al, Infect. Immun., 62:863-867 (1994).
Sjöstedt, A., G. Sandström, and A. Tärnvik. “Humoral and cell-mediated immunity in mice to a 17-kilodalton lipoprotein of Francisella tularensis expressed by Salmonella typhimurium.” Infection and immunity 60, no. 7 (1992): 2855-2862.
Snyder and Champness (eds.) Molecular Genetics of Bacteria. 3rd Ed. ASM Press: 2007.
Solanki, Amit Kumar, Bharati Bhatia, Himani Kaushik, Sachin K. Deshmukh, Aparna Dixit, and Lalit C. Garg. “Clostridium perfringens beta toxin DNA prime-protein boost elicits enhanced protective immune response in mice.” Applied microbiology and biotechnology 101, no. 14 (2017): 5699-5708. doi:10.1007/s00253-017-8333-2.
Spor et al., 2011, Unravelling the effects of the environment and host genotype on the gut microbiome, Nature Reviews Microbiology 9: 279-290
Spreng, Simone, Guido Dietrich, and Gerald Weidinger. “Rational design of Salmonella-based vaccination strategies.” Methods 38, no. 2 (2006): 133-143.
Srinivasan, Aparna, Joseph Foley, and Stephen J. McSorley. “Massive number of antigen-specific CD4 T cells during vaccination with live attenuated Salmonella causes interclonal competition.” The Journal of Immunology 172, no. 11 (2004): 6884-6893.
Stabel et al, Infect. Immun., 59:2941-2947 (1991).
Ståhl, Stefan, and Mathias Uhlén. “Bacterial surface display: trends and progress.” Trends in biotechnology 15, no. 5 (1997): 185-192.
Stevenson, Gordon, and Paul A. Manning. “Galactose epimeraseless (GalE) mutant G30 of Salmonella typhimurium is a good potential live oral vaccine carrier for fimbrial antigens.” FEMS microbiology letters 28, no. 3 (1985): 317-321.
Stocker, Bruce A D, and Salete M C Newton. “Immune responses to epitopes inserted in Salmonella flagellin.” International reviews of immunology 11, no. 2 (1994): 167-178.
Stocker, Bruce A D. “Novel non-reverting Salmonella live vaccines.” U.S. Pat. No. 4,735,801, issued Apr. 5, 1988.
Strindelius, Lena, Malin Filler, and Ingvar Sjöholm. “Mucosal immunization with purified flagellin from Salmonella induces systemic and mucosal immune responses in C3H/HeJ mice.” Vaccine 22, no. 27-28 (2004): 3797-3808.
Su F, Patel G B, Hu S, Chen W. Induction of mucosal immunity through systemic immunization: Phantom or reality? Human vaccines & immunotherapeutics. 2016; 12(4):1070-9.
Sugamata and Shiba, 2005 Applied and Environmental Microbiology 71: 656-662.
Surawicz, C. M. & Alexander, J. Treatment of refractory and recurrent Clostridium difficile infection. Nature Reviews Gastroenterology and Hepatology 8, doi:10.1038/nrgastro.2011.59 (2011).
Sztein M B. Cell-mediated immunity and antibody responses elicited by attenuated Salmonella enterica Serovar Typhi strains used as live oral vaccines in humans. Clinical infectious diseases: an official publication of the Infectious Diseases Society of America. 2007; 45 Suppl 1:S15-9.
Tacket et al, Infect. Immun., 60:536-541 (1992).
Tacket et al, Vaccine, 10:443-446 (1992).
Takata, Tetsuo, Toshiro Shirakawa, Yoshiko Kawasaki, Shohiro Kinoshita, Akinobu Gotoh, Yasunobu Kano, and Masato Kawabata. “Genetically engineered Bifidobacterium animalis expressing the Salmonella flagellin gene for the mucosal immunization in a mouse model.” The Journal of Gene Medicine: A cross-disciplinary journal for research on the science of gene transfer and its clinical applications 8, no. 11 (2006): 1341-1346.
Tanskanen et al. 2000, Appl. Env. Microbiol. 66: 4152-4156).
Thatte, Jayant, Satyajit Rath, and Vineeta Bal. “Immunization with live versus killed Salmonella typhimurium leads to the generation of an IFN-γ-dominant versus an IL-4-dominant immune response.” International immunology 5, no. 11 (1993): 1431-1436.
Tian, J.-H. H. et al. A novel fusion protein containing the receptor binding domains of C. difficile toxin A and toxin B elicits protective immunity against lethal toxin and spore challenge in preclinical efficacy models. Vaccine 30, 4249-4258, doi:10.1016/j.vaccine.2012.04.045 (2012).
Tite, J. P., X. M. Gao, C. M. Hughes-Jenkins, M. Lipscombe, D. O'Callaghan, G. Dougan, and F. Y. Liew. “Anti-viral immunity induced by recombinant nucleoprotein of influenza A virus. III. Delivery of recombinant nucleoprotein to the immune system using attenuated Salmonella typhimurium as a live carrier.” Immunology 70, no. 4 (1990): 540.
Toso J F, Gill V J, Hwu P, Marincola F M, Restifo N P, Schwartzentruber D J, Sherry R M, Topalian S L, Yang J C, Stock F, Freezer L J, Morton K E, Seipp C, Haworth L, Mavroukakis S, White D, MacDonald S, Mao J, Sznol M, Rosenberg S A. 2002. Phase I study of the intravenous administration of attenuated Salmonella typhimurium to patients with metastatic melanoma. Journal of clinical oncology: official J. of the Am. Society of Clinical Oncology 20:142-152.
Toussaint, Bertrand, Xavier Chauchet, Yan Wang, Benoit Polack, and Audrey Le Gouellec. “Live-attenuated bacteria as a cancer vaccine vector.” Expert review of vaccines 12, no. 10 (2013): 1139-1154.
Troy S B, Ferreyra-Reyes L, Huang C, Sarnquist C, Canizales-Quintero S, Nelson C, Baez, Saldana R, Holubar M, Ferreira-Guerrero E, Garcia-Garcia L, Maldonado Y A. 2014. Community circulation patterns of oral polio vaccine serotypes 1, 2, and 3 after Mexican national immunization weeks. J Infect Dis 209:1693-1699.
Turner et al, Infect. Immun., 61:5374-5380 (1993).
van Damme et al, Gastroenterol., 103:520-531 (1992).
Van Immerseel, Filip, U. Methner, I. Rychlik, B. Nagy, P. Velge, G. Martin, N. Foster, Richard Ducatelle, and Paul A. Barrow. “Vaccination and early protection against non-host-specific Salmonella serotypes in poultry: exploitation of innate immunity and microbial activity.” Epidemiology & Infection 133, no. 6 (2005): 959-978.
van Kleef, E., Deeny, S. R., Jit, M. & Vaccine, C.-B. The projected effectiveness of Clostridium difficile vaccination as part of an integrated infection control strategy. Vaccine (2016).
Veiga et al. 2003 Journal of Bacteriology 185: 5585-5590
Vendrell, Alejandrina, Claudia Mongini, Marìa José Gravisaco, Andrea Canellada, Agustina Inés Tesone, Juan Carlos Goin, and Claudia Inés Waldner. “An oral salmonella-based vaccine inhibits liver metastases by promoting tumor-specific T-cell-mediated immunity in celiac and portal lymph nodes: a preclinical study.” Frontiers in Immunology 7 (2016): 72).
Verma et al. 1995 Vaccine 13: 235-24.
Wahid R, Pasetti M F, Maciel M, Jr., Simon J K, Tacket C O, Levine M M, et al. Oral priming with Salmonella Typhi vaccine strain CVD 909 followed by parenteral boost with the S. Typhi Vi capsular polysaccharide vaccine induces CD27+IgD-S. Typhi-specific IgA and IgG B memory cells in humans. Clinical immunology (Orlando, Fla.). 2011; 138(2):187-200.
Walker, Mark J., Manfred Rohde, Kenneth N. Timmis, and Carlos A. Guzman. “Specific lung mucosal and systemic immune responses after oral immunization of mice with Salmonella typhimurium aroA, Salmonella typhi Ty21a, and invasive Escherichia coli expressing recombinant pertussis toxin S1 subunit.” Infection and immunity 60, no. 10 (1992): 4260-4268.
Wang J Y, Harley R H, Galen J E. Novel methods for expression of foreign antigens in live vector vaccines. Human vaccines & immunotherapeutics. 2013; 9(7):1558-64.
Wang, Shifeng, Qingke Kong, and Roy Curtiss III. “New technologies in developing recombinant attenuated Salmonella vaccine vectors.” Microbial pathogenesis 58 (2013): 17-28.
Wang, Shifeng, Yuhua Li, Huoying Shi, Wei Sun, Kenneth L. Roland, and Roy Curtiss. “Comparison of a regulated delayed antigen synthesis system with in vivo-inducible promoters for antigen delivery by live attenuated Salmonella vaccines.” Infection and immunity 79, no. 2 (2011): 937-949.
Wang, Yuanguo; Wang, Shaohui; Bouillaut, Laurent; Li, Chunhui; Duan, Zhibian; Zhang, Keshan; Tzipori, Saul; Sonenshein, Abraham; Sun, Xingmin. (2018). Oral immunization with non-toxic C. difficile strains expressing chimeric fragments of TcdA and TcdB elicits protective immunity against C. difficile infection in both mice and hamsters. Infection and Immunity. 10.1128/IAI.00489-18.
Ward, Stephen J., Gill Douce, Dayse Figueiredo, Gordon Dougan, and Brendan W. Wren. “Immunogenicity of a Salmonella typhimurium aroA aroD vaccine expressing a nontoxic domain of Clostridium difficile toxin A.” Infection and immunity 67, no. 5 (1999): 2145-2152.
Warren, C. A. et al. Amixicile, a novel inhibitor of pyruvate: ferredoxin oxidoreductase, shows efficacy against Clostridium difficile in a mouse infection model. Antimicrob. Agents Chemother. 56, 4103-4111, doi:10.1128/AAC.00360-12 (2012).
Wei et al. 2008 J. Virology 82: 6200-6208
Wells, J. M., K. Robinson, L. M. Chamberlain, K. M. Schofield, and R. W. F. Le Page. “Lactic acid bacteria as vaccine delivery vehicles.” Antonie Van Leeuwenhoek 70, no. 2-4 (1996): 317-330.
Wick et al, Infect. Immun., 62:4542-4548 (1994).
Wieckowski, Sebastien, Lilli Podola, Heiko Smetak, Anne-Lucie Nugues, Philippe Slos, Amine Adda Berkane, Ming Wei et al. “Modulating T cell immunity in tumors by targeting PD-L1 and neoantigens using a live attenuated oral Salmonella platform.” (2018): 733-733.
Wieckowski, Sebastien, Lilli Podola, Marco Springer, Iris Kobl, Zina Koob, Caroline Mignard, Amine Adda Berkane et al. “Immunogenicity and antitumor efficacy of live attenuated Salmonella typhimurium-based oral T-cell vaccines VXM01m, VXM04m and VXM06m.” (2017): 4558-4558.
Wieckowski, Sebastien, Lilli Podola, Marco Springer, Iris Kobl, Zina Koob, Caroline Mignard, Alan Broadmeadow et al. “Non-clinical safety, immunogenicity and antitumor efficacy of live attenuated Salmonella Typhimurium-based oral T-cell vaccines VXM01m, VXM04m and VXM06m.” In Molecular Therapy, vol. 25, no. 5, pp. 360-360. 50 Hampshire St, Floor 5, Cambridge, Mass. 02139 USA: Cell Press, 2017.
Wiegand, P. N. et al. Clinical and economic burden of Clostridium difficile infection in Europe: a systematic review of healthcare-facility-acquired infection. J. Hosp. Infect. 81, 1-14, doi:10.1016/j.jhin.2012.02.004 (2012).
Wilcox, Mark H., Dale N. Gerding, Ian R. Poxton, Ciaran Kelly, Richard Nathan, Thomas Birch, Oliver A. Comely et al. “Bezlotoxumab for prevention of recurrent Clostridium difficile infection.” New England Journal of Medicine 376, no. 4 (2017): 305-317. doi:10.1056/NEJMoa1602615.
Winter, K, Xing, L. and Ward, B. G., McGill University, “Attenuated Salmonella typhimurium as a vector for a novel Clostridium difficile vaccine”, Abstract II 084, CSM 2017 Poster Session, 67th Annual Conference of the Canadian Society of Microbiologists, University of Waterloo, Waterloo, Ontario, Jun. 20-Jun. 23, 2017.
Witworth et al., 2005, Infect. Immun. 73:6668-6673
Wooldridge, K. (ed) Bacterial Secreted Proteins. Caster Academic Press 2009.
Wu, Jane Y., Salete Newton, Amrit Judd, Bruce Stocker, and William S. Robinson. “Expression of immunogenic epitopes of hepatitis B surface antigen with hybrid flagellin proteins by a vaccine strain of Salmonella.” Proceedings of the National Academy of Sciences 86, no. 12 (1989): 4726-4730.
Wu et al., 1989 Proc. Natl. Acad. Sci. USA 86: 4726-4730.
Wyszyfiska, Agnieszka, Patrycja Kobierecka, Jacek Bardowski, and Elzbieta Katarzyna Jagusztyn-Krynicka. “Lactic acid bacteria—20 years exploring their potential as live vectors for mucosal vaccination.” Applied microbiology and biotechnology 99, no. 7 (2015): 2967-2977.
Xiong G, Husseiny M I, Song L, Erdreich-Epstein A, Shackleford G M, Seeger R C, et al. Novel cancer vaccine based on genes of Salmonella pathogenicity island 2. International journal of cancer. 2010; 126(11):2622-34, doi:10.1002/ijc.24957 (2010).
Xu, Fengfeng, Mei Hong, and Jeffrey B. Ulmer. “Immunogenicity of an HIV-1 gag DNA vaccine carried by attenuated Shigella.” Vaccine 21, no. 7-8 (2003): 644-648.
Xu, Yigang, and Yijing Li. “Induction of immune responses in mice after intragastric administration of Lactobacillus casei producing porcine parvovirus VP2 protein.” Applied and environmental microbiology 73, no. 21 (2007): 7041-7047.
Yam K K, Gupta J, Winter K, Allen E, Brewer A, Beaulieu E, et al. AS03-Adjuvanted, Very-Low-Dose Influenza Vaccines Induce Distinctive Immune Responses Compared to Unadjuvanted High-Dose Vaccines in BALB/c Mice. Frontiers in immunology. 2015; 6:207.
Yang et al, J. Immunol., 145:2281-2285 (1990).
Yen C, Jakob K, Esona M D, Peckham X, Rausch J, Hull J J, Whittier S, Gentsch J R, LaRussa P. 2011. Detection of fecal shedding of rotavirus vaccine in infants following their first dose of pentavalent rotavirus vaccine. Vaccine 29:4151-4155.
Yoon et al., 2010 Secretory production of recombinant proteins in Escherichia coli, Recent Patents on Biotechnology 4: 23-29.
Zegers, N. D., E. Kluter, H. van Der Stap, E. Van Dura, P. Van Dalen, M. Shaw, and L. Baillie. “Expression of the protective antigen of Bacillus anthracis by Lactobacillus casei: towards the development of an oral vaccine against anthrax.” Journal of applied microbiology 87, no. 2 (1999): 309-314.
Zhang et al., 2006, Extracellular accumulation of recombinant proteins fused to the carrier protein YebF in Escherichia coli, Nat Biotechnol 24: 100-104
Zhang, Ling, Lifang Gao, Lijuan Zhao, Baofeng Guo, Kun Ji, Yong Tian, Jinguo Wang et al. “Intratumoral delivery and suppression of prostate tumor growth by attenuated Salmonella enterica serovar typhimurium carrying plasmid-based small interfering RNAs.” Cancer research 67, no. 12 (2007): 5859-5864.
Zhang, Shanshan, Sarah Palazuelos-Munoz, Evelyn M. Balsells, Harish Nair, Ayman Chit, and Moe H. Kyaw. “Cost of hospital management of Clostridium difficile infection in United States—a meta-analysis and modelling study.” BMC infectious diseases 16(1) (2016): 447.
Zhao, Zhanqin, Yun Xue, Bin Wu, Xibiao Tang, Ruiming Hu, Yindi Xu, Aizhen Guo, and Huanchun Chen. “Subcutaneous vaccination with attenuated Salmonella enterica serovar Choleraesuis C500 expressing recombinant filamentous hemagglutinin and pertactin antigens protects mice against fatal infections with both S. enterica serovar Choleraesuis and Bordetella bronchiseptica.” Infection and immunity 76, no. 5 (2008): 2157-2163.
Zian et al., 2008, Proteome-Based Identification of Fusion Partner for High-Level Extracellular Production of Recombinant Proteins in Escherichia coli, Biotechnol Bioegineer 101: 587-601.

### SUMMARY OF THE INVENTION

Clostridium difficile diarrhea is a toxin-mediated disease. Expressed by most clinical isolates, toxins A and B (TcdA and TcdB) can both damage the gastrointestinal epithelium. The receptor binding domains (RBD) of these toxins are immunogenic and antibodies against the RBDs are neutralizing. Since these toxins act locally, an optimal C. difficile vaccine would generate both systemic and mucosal responses. A highly attenuated Salmonella typhimurium strain (YS1646), originally developed as a cancer treatment, is used to produce such a vaccine. Promoters and secretory signals from Type 3 secretion systems of S. typhimurium as well as constitutive promoters were screened to generate plasmid-based candidates that express either the TcdA or TcdB RBD.

Different vaccine routes and schedules were tested to achieve detectable serum and mucosal antibody titers in C57BL/6J mice with the shortest possible delay [e.g., recombinant RBD intramuscular (IM), oral (PO), multi-modality (IM+PO), prime-pull (IM then PO), 1, 2 and 4 week schedules]. All of the different routes and schedules were well-tolerated by the mice. Several TcdA or TcdB candidates were identifed that can provide 100% protection against lethal challenge when given in a multi-modality schedule over 1 week (day 0 IM+PO, days 3 and 5 PO). Substantial protection (82%) was achieved with combined PO TcdA/TcdB vaccination alone (d1, 3 and 5 difficile and strongly support their further development.

Several groups have demonstrated the potential of oral vaccines to elicit protective responses to RBDs in animal models of CDI. For example, Guo et al demonstrated that oral administration of Lactococcus lactis expressing both the RBDs of TcdA and TcdB could elicit both IgA and IgG and protect mice from lethal challenge (Guo et al. 2015). In conceptually similar studies, Hong and colleagues showed that hamsters given Bacillus subtilis spores expressing the carboxy-terminal segment of TcdA orally (Wang et al. 2018) can be protected from C. difficile colonization by mucosal IgA (Hong et al. 2017). A locally-invasive but highly attenuated Salmonella typhimurium vector might be even more effective in the induction of local and systemic anti-RBD responses. The flagellin protein of S. typhimurium has been proposed as a general mucosal adjuvant through its action on toll-like receptor (TLR) 5 (Makvandi et al. 2018). The S. typhimurium flagellin protein (Flic) fused to TcdA or TcdB can elicit toxin-specific IgA and IgG and protect mice from lethal challenge (Ghose et al. 2013). Other Salmonella products such as lipopolysaccharide (LPS) would be expected to further enhance immune responses by triggering additional pathogen recognition receptors (PRRs: TLR4) (Hayashi et al. 2001). Indeed, live attenuated Salmonella have multiple potential advantages as vaccine vectors. They directly target the intestinal M cells overlying the gut-associated lymphoid tissues (GALT) (Jepson et al. 2001) leading to the induction of both humoral and cellular responses to their foreign protein ‘cargo’ (Penha Filho et al. 2012). They also have a large ‘carrying’ capacity and are easy to manipulate both in the laboratory and at industrial scale.

that are expressed, secreted, surface displayed and/or released by bacteria and result in immunologic activity, and may optionally include the combination with secreted protease inhibitors. The bacterial delivery vector may be attenuated, non-pathogenic, low pathogenic (including wild type), or a probiotic bacterium. The bacteria are introduced either systemically (e.g., parenteral, intravenous (IV), intramuscular (IM), intralymphatic (IL), intradermal (ID), subcutaneously (sub-q), local-regionally (e.g., intralesionally, intratumorally (IT), intrapaeritoneally (IP), topically, intrathecally (intrathecal), by inhaler or nasal spray) or to the mucosal system through oral, nasal, pulmonary intravessically, enema or suppository administration where they are able to undergo limited replication, express, surface display, secrete and/or release the antigenic proteins or a combination thereof, and thereby provide a therapeutic or preventive benefit.

Promoters, i.e., genetic regulatory elements that control the expression of the genes encoding the therapeutic molecules described above that are useful in the present technology, according to various embodiments, include constitutive and inducible promoters. A preferred constitutive promoter is that from the vector pTrc99a (Promega). Preferred inducible promoters include the tetracycline inducible promoter (TET promoter), colicin promoters, sulA promoters and hypoxic-inducible promoters including but not limited to the PepT promoter (Bermudes et al., WO 01/25397), the arabinose inducible promoter (AraBAD) (Lossner et al. 2007; WO/2006/048344) the salicylate (aspirin) derivatives inducible promoter (Royo et al. 2007; WO/2005/054477), or a quorum-sensing (autoinduction) promoter (Anerson et al., 2006).

A single promoter may be used to drive the expression of more than one gene, such as an antigen and a protease inhibitor. The genes may be part of a single synthetic operon (polycistronic), or may be separate, monocystronic constructs, with separate individual promoters of the same type used to drive the expression of their respective genes. The promoters may also be of different types, with different genes expressed by different constitutive or inducible promoters. Use of two separate inducible promoters for more than one antigen or other effector type peptide allows, when sufficient tetracycline, arabinose or salicylic acid is administered following administration of the bacterial vector, their expression to occur simultaneously, sequentially, or alternatingly (i.e., repeated). An inducible promoter is not required, and a constitutive promoter may be employed.

Salmonella type-III secretion systems (T3SS) and both T3SS-specific and constitutive promoters and secretory signals were exploited to generate 15 YS1646 strains with plasmid-based expression of the RBD portion of either TcdA or TcdB. These strains were screened for protein expression in axenic culture and murine RAW 264 murine macrophages. The most promising constructs were advanced to immunogenicity testing in adult female C57BL/6 mice using different routes (e.g., recombinant protein IM, YS1646 strains orally (PO)) and schedules (e.g., repeat dosing, multi-modality, prime-pull) to achieve the best serologic response in the shortest period of time. Two of the YS1646 strains elicited strong systemic IgG and intestinal IgA responses and provided up to 100% protection from lethal challenge when administered in a multi-modality schedule over 5 days (IM+PO on day 1 followed by PO boosting on days 3 and 5).

It is therefore an object to provide pharmaceutically acceptable orally-administrable vaccine formulation, comprising: an attenuated recombinant Salmonella bacterium adapted for colonization of a human gut, expressing at least one antigen corresponding to at least one of C. difficile TcdA and TcdB receptor binding domains; and a pharmaceutically acceptable carrier adapted to preserve the attenuated Salmonella bacterium through the gastrointestinal tract for delivery in the human gut.

The at least one antigen may secreted from the Salmonella bacteria by a Salmonella Type 3 secretion system.

The at least one antigen may be selected from the group consisting of at least one of TcdA₅₄₅₈₋₈₁₃₀ and TcdB₅₄₆₁₋₇₀₈₀.

The at least one antigen may be expressed in a fusion peptide with a secretory signal selected from the group consisting of one or more of SopE2, SseJ, SptP, SspH1, SspH2, SteA, and SteB.

The transcription of the at least one antigen may be under control of at least one promoter selected from the group consisting of one or more of SopE2, SseJ, SptP, SspH1, SspH2, SteA, SteB, pagC, lac, nirB, and pagC.

The at least one antigen may be produced based on a chromosomally integrated genetically engineered construct and/or a plasmid genetically engineered construct.

The at least one antigen may be produced based on a genetically engineered construct comprising a promoter portion, a secretion signal portion, and an antigen portion.

The promoter portion and the secretion signal portion may be separated by a first restriction endonuclease cleavage site. The secretion signal portion and the antigen portion may also be separated by a second restriction endonuclease cleavage site.

The genetically engineered construct may comprise plasmid, further comprising an antibiotic resistance gene.

It is another object to provide a recombinant attenuated Salmonella bacterium adapted for growth in a mammal, expressing at least one antigen corresponding to a C. difficile antigen, adapted to induce a vaccine response to C. difficile after oral administration to the mammal.

It is another object to provide a recombinant attenuated Salmonella bacterium adapted for growth in a mammal, expressing at least one antigen corresponding to at least one of C. difficile TcdA and TcdB receptor binding domains, adapted to induce an vaccine response to C. difficile after oral administration to the mammal.

It is a further object to provide a method of immunizing a human against infection by C. difficile, comprising orally administering a pharmaceutically acceptable formulation, comprising: an attenuated recombinant Salmonella bacterium, expressing at least one antigen corresponding to at least one of C. difficile TcdA and TcdB receptor binding domains; and a pharmaceutically acceptable carrier adapted to preserve the attenuated Salmonella bacterium through the gastrointestinal tract for delivery in the human gut.

The method may further comprise administering a second pharmaceutically acceptable formulation comprising at least one antigen corresponding to at least one of C. difficile TcdA and TcdB receptor binding domains through a non-oral route of administration.

The non-oral route of administration may comprise an intramuscular route of administration. The second pharmaceutically acceptable formulation may comprise an adjuvant.

The administration of the first pharmaceutically acceptable formulation and second pharmaceutically acceptable formulation may be concurrent, or the first pharmaceutically acceptable formulation may precede or succeeds the administering of the second pharmaceutically acceptable formulation. The administering of the first and/or second pharmaceutically acceptable formulation may be dependent on a test of pre-existing immunity of the human.

The administering of the first pharmaceutically acceptable formulation and the second pharmaceutically acceptable formulation may be according to a prime-pull, prime-boost or alternate administration protocol.

The administering of the first pharmaceutically acceptable formulation and the second pharmaceutically acceptable formulation may be in a manner dependent on tests of at least IgG and IgA immune response.

The administering of the first pharmaceutically acceptable formulation and the second pharmaceutically acceptable formulation are preferably effective to produce both IgG and IgA immunity to C. difficile.

It is an object to provide a vaccine adapted to raise immunity to C. difficile in animals, comprising an attenuated recombinant bacterium adapted to secrete a C. difficile antigen, e.g., TcdA or TcdB.

The attenuated recombinant bacterium may be YS1646 or YS1646 zwf-.

The vaccine may be provided in a kit with an i.m. dosage form of the C. difficile antigen, or adjuvanted C. difficile antigen.

It is a further object to provide a method of immunizing an animal against C. difficile, comprising enterically administering at least one dose of a live attenuated recombinant bacterium genetically engineered to secrete a C. difficile antigen in the animal's gut.

The method may further comprise parenterally administering at least one dose of a purified C. difficile antigen to the animal, e.g., prior to enterically administering the live attenuated recombinant bacterium.

The at least one dose of a C. difficile antigen may be provided in a dosage form comprising an adjuvant.

The animal may be uninfected with C. difficile, and the animal may develop a preventative immune response to C. difficile. The animal may be infected with C. difficile, and the animal may develop a therapeutic immune response to C. difficile.

The enteric administration of at least one dose of a live attenuated recombinant bacterium genetically engineered to secrete the C. difficile antigen in the animal's gut may be repeated at least once, e.g., at least twice, with at least 24 hours between doses. The enteric administration may be preceded by at least one parenteral dose of the C. difficile antigen, and the enterically administering may be thereafter repeated at least once with at least 24 hours between enteric doses.

The pharmaceutically acceptable orally-administrable vaccine formulation may produce at least one antigen is produced based on a chromosomally-integrated genetically engineered construct.

The genetically engineered Salmonella may include a chromosomally-integrated genetically engineered construct which is genetically stabilized by delection of at least one IS200 element.

A further object provides a kit for immunizing a human against C. difficile, comprising a first pharmaceutically acceptable formulation, comprising a live attenuated recombinant Salmonella bacterium, expressing at least one antigen corresponding to at least one of C. difficile TcdA and TcdB receptor binding domains adapted for oral administration; and a second pharmaceutically acceptable formulation comprising at least one purified antigen corresponding to at least one of C. difficile TcdA and TcdB receptor binding domains adapted for a non-oral route of administration. The first pharmaceutically acceptable formulation may have an enteric release coating, and the second second pharmaceutically acceptable formulation may include an adjuvant. The kit may further comprise a lateral flow assay strip for determining an immune status of a patient with respect to C. difficile and/or the vaccine antigen(s).

The compositions and methods described herein can be administered to a subject in need of treatment, e.g., having a risk of infection, or an existing infection. In some embodiments, the methods described herein comprise administering an effective amount of compositions described herein, e.g. engineered microbial cells to a subject in order to alleviate a symptom. As used herein, “alleviating a symptom” is ameliorating any condition or symptom associated with a given condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, subcutaneous, transdermal, airway (aerosol), cutaneous, topical, or injection administration. Administration can be local or systemic.

The term “effective amount” as used herein refers to the amount of engineered microbial cells needed to alleviate at least one or more symptom of the disease or disorder, and relates to a sufficient amount of pharmacological composition to provide the desired effect. The term “therapeutically effective amount” therefore refers to an amount of engineered microbial cells that is sufficient to effect a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio ED₅o. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of an engineered microbial cell which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for inflammation, among others. The dosage can be determined by a physician and adjusted, as necessary, to suit observed effects of the treatment.

In some embodiments, the technology described herein relates to a pharmaceutical composition comprising an engineered microbial cell and/or purified antigen as described herein, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutical compositions comprising an engineered microbial cell can be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

In certain embodiments, an effective dose of a composition comprising engineered microbial cells as described herein can be administered to a patient once. In certain embodiments, an effective dose of a composition comprising engineered microbial cells can be administered to a patient repeatedly. In some embodiments, the dose can be a daily administration, for example oral administration, of, e.g., a capsule comprising bacterial cells as described herein. In some embodiments, the dose can be, e.g. an injection or gavage of bacterial cells. In some embodiments, the dose can be administered systemically, e.g. by intravenous injection. In some embodiments, a dose can comprise from 10⁶ to 10¹² cells. In some embodiments, a dose can comprise from about 10⁸ to 10¹⁰ cells. A composition comprising engineered microbial cells can be administered over a period of time, such as over a 5 minute, 10 minute, 15 minute, 20 minute, or 25 minute period. The administration can be repeated, for example, on a regular basis, such as every few days, once a week, or biweekly (i.e., every two weeks) for one month, two months, three months, four months or longer.

In some embodiments, after an initial treatment regimen, the treatments can be administered on a less frequent basis. For example, after treatment biweekly for three months, treatment can be repeated once per month, for six months or a year or longer.

The efficacy of engineered microbial cells in, e.g. the raising of an appropriate immune response to a specified disease, e.g., C. difficile, can be determined by the skilled clinician. However, a treatment is considered “effective treatment,” as the term is used herein, clinically useful partial or complete immunity is achieved. Efficacy can be assessed, for example, by measuring a marker, indicator, population statistic, or any other measurable parameter appropriate.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, the terms “reduced”, “reduction”, “decrease”, or “inhibit” can mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or more or any decrease of at least 10% as compared to a reference level. In some embodiments, the terms can represent a 100% decrease, i.e., a non-detectable level as compared to a reference level. In the context of a marker or symptom, a “decrease” is a statistically significant decrease in such level. The decrease can be, for example, at least 10%, at least 20%, at least 30%, at least 40% or more, and is preferably down to a level accepted as within the range of normal for an individual without such disorder. In some instances, the symptom can be essentially eliminated which means that the symptom is reduced, i.e., the individual is in at least temporary remission.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or non-human animal. Usually the non-human animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Animals also include armadillos, hedgehogs, and camels, top name a few. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, cow, or pig, but is not limited to these examples. Mammals other than humans can be advantageously used as subjects that represent animal models of a given condition. A subject can be male or female.

A subject can be one who has been previously diagnosed with or identified as suffering from or having a condition in need of treatment, and optionally, have already undergone treatment. Alternatively, a subject can also be one who has not been previously diagnosed as having a condition. For example, a subject can be one who exhibits one or more risk factors or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operatively linked to appropriate regulatory sequences. A gene may or may not include regions preceding and following the coding region, e.g. 5′ untranslated (5′UTR) or “leader” sequences and 3′ UTR or “trailer” sequences.

The term “operatively linked” includes having an appropriate start signal (e.g., ATG) in front of the polynucleotide sequence to be expressed, and maintaining the correct reading frame to permit expression of the polynucleotide sequence under the control of the expression control sequence, and, optionally, production of the desired polypeptide encoded by the polynucleotide sequence. In some examples, transcription of a nucleic acid is under the control of a promoter sequence (or other transcriptional regulatory sequence) which controls the expression of the nucleic acid in a cell-type in which expression is intended. It will also be understood that the nucleic acid can be under the control of transcriptional regulatory sequences which are the same or which are different from those sequences which control transcription of the naturally-occurring form of a protein.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.”

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. infection. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%. More generally, the word “about” means that the semantic or quantitative parameter achieves the stated function to achieve the corresponding benefit, or within a medically acceptable range, achieves a substantial portion of the benefit and is used in conjunction with another therapy to achieve a similar functional benefit. Thus, if a vaccine comprising “about” 10⁸ cfu of bacteria is specified as achieving blocking immunity, a lesser amount or benefit may be encompassed if the specified vaccine substantially contributes to achieving the blocking immunity, while another vaccine or means of achieving the same result is provided to supplement the deficiency, and the specified vaccine alone is capable of achieving the specified immunity.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in (Merck Research Laboratories, 2006; Porter et al. 1994; Lewin 2009; Kendrew et al. 1995; and Coligan et al. 2009). Unless otherwise stated, the present invention was performed using standard procedures, as described, for example in (Sambrook et al. 2001; Davis et al., 1995; Coligan, et. al. 2009; Bonifacino et. al. 2001; Freshney 2005; Mather et al. 1998). Other terms are defined herein within the description of the various aspects of the invention.

All patents and other publications; including literature references, issued patents, published patent applications, and co-pending patent applications; cited throughout this application are expressly incorporated herein by reference for all purposes, including, but not limited to, describing and disclosing, for example, the methodologies described in such publications that might be used in connection with the technology described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize.

For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### BRIEF DESCRIPTION OF THE DRAWINGS

FIG. 1 shows a generic map of plasmids constructed and used.

FIGS. 2A-2E show photomicrographs of transformed YS1646 strains expressed heterologous antigen.

FIGS. 3A and 3B show results of vaccination with antigen expressing YS1646 strains protected against C. difficile challenge.

FIGS. 4A-4F show that vaccination with antigen expressing YS1646 increases antibody titers in intestines mice pre-challenge and in post-challenge survivors.

### DETAILED DESCRIPTION OF THE PREFERRED EMBODIMENTS

### Methods

Bacterial Strains and Growth Conditions Salmonella enterica Typhimurium YS1646 (DmsbB2 DpurI DSuwwan xyl⁻) (ATCC 202165: ATCC, Manassas, Va.) was obtained from Cedarlane Labs (Burlington, ON). Escherichia coli DH5a (ThermoFischer Scientific, Eugene, Oreg.) was used for production of recombinant plasmids. Plasmids were introduced into E. coli or YS1646 by electroporation (2 ag of plasmid at 3.0 kV, 200 W, and 25 μF) (GenePulser XCell, Bio-Rad, Hercules, Calif., USA). Transformed bacteria were grown in Luria Broth (LB) with 50 μg/mL of ampicillin (Wisent, St. Bruno, QC) for YS1646 or 30 μg/ml of kanamycin (Wisent) for E. coli. Clostridium difficile Strain VPI 10463 (ATCC 43255) was obtained from Cedarlane Labs and used for challenge experiments. Cells were maintained in meat broth (Sigma-Aldrich, St Louis, Mo.) containing 0.1% (w/v) L-cysteine (Sigma-Aldrich) in an anaerobic jar. For colony counts, C. difficile containing media was serially diluted and streaked onto pre-reduced Brain Heart Infusion (BHIS) plates (BD Biosciences, Mississauga, ON), containing 0.1% (w/v) L-cysteine. Plates were left to grow at 37° C. in an anaerobic jar overnight.

Plasmid Construction

Vaccine Candidate Plasmids

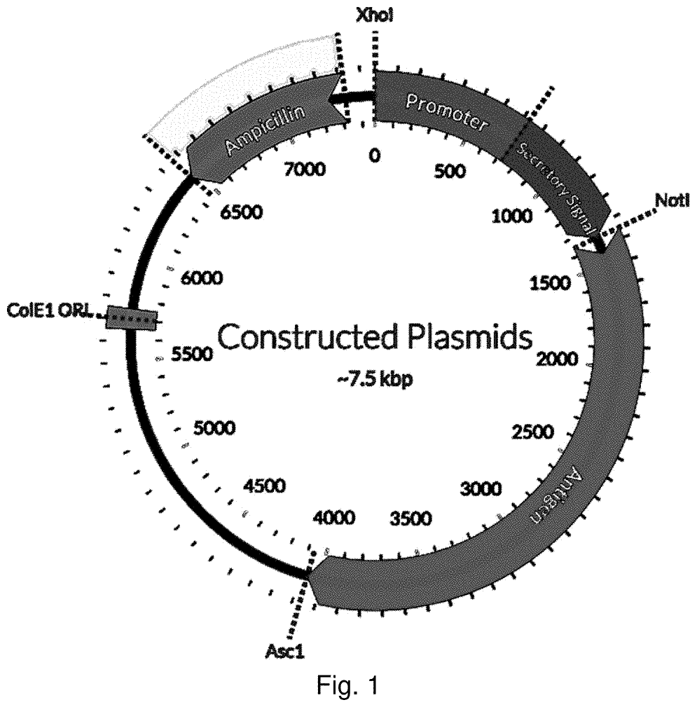

The pQE_30 plasmid (Qiagen, Venlo, Limburg, Netherlands) backbone containing an ampicillin resistance gene used for antigen expression in the vaccine candidates was cloned from the plasmid roGFP_IL_pQE30, a gift from David Ron (Addgene, plasmid #48633) (Azevov et al. 2013). PCR was used to obtain the SopE2, SptP, SseJ, SspH1, SspH2, SteA and SteB promoter and secretory signal sequences from YS1646. The PagC promoter from YS1646 and the nirB promoter from E coli were also PCR amplified. The lac promoter was incorporated into the 5′ PCR primer. The antigenic C-terminal ends of the Receptor Binding domains for Toxin B (TcdB1821-2366) and Toxin A (TcdA1820-2710) were amplified by PCR from C. difficile VPI 10463. Restriction sites were incorporated 5′ of the promoters (Xho1), between the secretory signal and the antigen (Not1), and at the 3′ end of the antigen sequence (AscI). (FIG. 1) Primers used are listed in Table 1. DNA sequencing confirmed that plasmids had the expected sequence (McGill University Genome Centre, Montreal, QC). EGFP antigen was cloned from the plasmid pEGFP_C1 (Clontech, Mountain View, Calif.) with the Not1 and Asc1 incorporated in the primers. All plasmids are named based on the promoter, secretory signal and antigen used, these are described in Table 1. The unedited pQE_30 plasmid was transformed into YS1646 as a control and is referred to as pQE_null.

FIG. 1 shows a generic map of plasmids constructed and used in this study. The pQE_30 plasmid containing an ampicillin resistance gene was used as the plasmid backbone. The Promoter and secretory signals were inserted between XhoI and NotI digestive sites. The antigenic sequence was inserted between NotI and AscI digestive sites. Plasmids were between 3.4 kbp (pQE_null), and 7.5 kbp in size.

Recombinant TcdA and TcdB Expression

Protein expression and purification of recombinant TcdA1820-2710 (rbdA) and TcdB1821-2366 (rbdB) was accomplished using the pET-28b plasmid (Novagen, Millipore Sigma, Burlington, Mass.), with an Isopropyl-J3-D-1-thiogalactopyranoside (IPTG) inducible promoter and kanamycin resistance gene. A 6×His tag and stop codon was added at the 3′ end. The expression vector was transformed into E coli C25661 (New England BioLabs, Whitby, ON) as above. Transformed bacteria were grown in a 37° C. shaking incubator until the absorbance at 600 nm (OD600) reached 0.5-0.6. IPTG (Invitrogen, Carlsbad, Calif.) was then added and expression was induced for 3-4 hours. Cells were pelleted by centrifugation at 3000×g for 10 minutes at 4° C. Cells were lysed, and lysate was collected and purified using Ni-NTA affinity chromatography (Ni-NTA Superflow by Qiagen, Venlo, Limburg, Netherlands). The eluate was analyzed by Coomassie blue staining of polyacrylamide gels and Western Blot using a monoclonal antibody directed against the His-tag (Sigma-Aldrich).

Macrophage Infection

RAW 264.7 cells (ATCC TIB-71) were cultured in Dulbecco's Modified Eagle's Medium (DMEM) (Wisent) supplemented with 10% fetal bovine serum (FBS) and penicillin (100 000 U/mL) streptomycin (100 μg/mL] (Wisent); cells were passaged when they reached ˜90% confluence. For each passage, cells were washed with Hank's Balanced Salt Solution (HBSS) without calcium and magnesium (Wisent) and detached from the flasks using 0.25% Trypsin (Wisent). RAW 264.7 cells were seeded in Falcon™ Polystyrene 12-well plates (Corning Inc., Corning, N.Y.) at a density of 1×10⁶ cells/well for infection experiments 24 hours later. RAW 264.7 cells were infected at a multiplicity of infection (MOI) of either 40 or 100. For western blotting, cells were then incubated at 37° C. in 0% CO₂, as YS1646 is sensitive to increased CO₂ levels. Infection was allowed to proceed for an hour then cells were washed 3× with PBS and resuspended in DMEM with 50 μg/mL of gentamicin (Wisent) was added, to kill extracellular YS1646. After 2 hours, the gentamicin concentration was lowered to 5 μg/mL.

Fluorescence (EGFP) Microscopy

RAW 264.7 cells, plated on 8-well microscope chamber-slides (Eppendorf, Hamburg, Germany) at 1.8×10⁵ cells/chamber, were infected at a MOI of 40 with YS1646 strains transformed with the EGFP constructs. Infected cells were incubated at 37° C. in 5% CO₂. 24 hours after infection, cells were stained with 4′,6-diamidino-2-phenylindole (DAPI) (ThermoFischer Scientific) and fixed with 4% paraformaldehyde (Sigma Aldrich). A Zeiss LSM780 laser scanning confocal microscope was used for imaging (405 nm laser for excitation of DAPI, 488 nm laser for excitation of EGFP) and acquisition and processing was performed using ZEN software (Zeiss, Toronto, ON).

Western Blot

For antigen expression in axenic culture, transformed YS1646 strains were grown overnight in LB with 50 μg/mL of ampicillin and 0% CO2, centrifuged at 21 130×g for 10 minutes, resuspended in PBS, then mixed in with NuPAGE Lithium Dodecyl Sulfate (LDS) sample buffer (Invitrogen) according to the manufacturer's instructions. For antigen expression in RAW264.7 macrophages, infection was allowed to proceed for either 1 hour or 24 hours. Samples were then collected, centrifuged, resuspended in PBS, and mixed with sample buffer as above. All samples were heated for 10 min at 70° C., then cooled on ice. Proteins were separated on a 4-12% Bis-Tris Protein Gel (Invitrogen) and transferred to nitrocellulose membranes using the Trans-Blot® Turbo™ RTA Mini Nitrocellulose Transfer Kit (Bio-Rad, Hercules, Calif.). For detection of TcdA₅₄₅₈₋₈₁₃₀ and TcdB₅₄₆₁₋₇₀₈₀, the membranes were incubated first with anti-ToxinA chicken IgY (Abnova, Taipei, Taiwan) (1:5,000) and anti-ToxinB chicken IgY antibodies (1:10,000) (Abnova), respectively followed by goat anti-chicken IgY conjugated to horseradish peroxidase (1:10,000) (ThermoFisher Scientific). Immunoreactive bands were visualized using SuperSignal™ West Pico PLUS Chemiluminescent Substrate (ThermoFisher Scientific) and autoradiography film (Denville Scientific, Holliston, Mass.).

Mice

6 to 8-week-old female C57BL/6J mice were obtained from Charles River Laboratories (Montreal, QC) and were kept in pathogen-free conditions in the Animal Resource Division at the McGill University Health Center Research Institute (RI-MUHC). All animal procedures were approved by the Animal Care Committee of McGill University and performed in accordance with the guidelines of the Canadian Council on Animal Care.

Vaccination

For oral vaccinations, mice were gavaged with 1×10⁹ cfu of YS1646 strains in 0.2 ml of PBS (e.g., day 0, 2 and 4). When both strains were given, 5×10⁸ cfu of each strain was used, for a total of 1×10⁹ cfu of YS1646 given in 0.2 ml of PBS. Intramuscular (IM) injections contained a total of 10 μg of recombinant protein and 250 μg of Alhydrogel (alum) (Brenntag BioSector A/S, Frederikssund, Denmark) in 50 μL administered into the gastrocnemius muscle using a 28G needle.

Blood and Intestine Sampling

Baseline serum samples were collected from the lateral saphenous vein prior to all other study procedures using microtainer serum separator tubes (Sarstedt, Nümbrecht, Germany). Serum samples were also collected at the end of the study by cardiac puncture in mice after isofluorane/CO₂ anesthesia. Serum separation was performed according to manufacturer's instructions and aliquots were stored at −20° C. until used. At study termination, 10 cm of the small intestine, starting at the stomach, was collected. Intestinal contents were removed, and the tissue was weighed and stored in a Protease Inhibitor (PI) Cocktail (Sigma Aldrich-P8340) at a 1:5 dilution (w/v) on ice until processed. The tissue was homogenized (Homogenzier 150; Fisher Scientific, Ottawa, ON), centrifuged at 2500×g at 4° C. for 30 minutes and the supernatant was collected. Supernatants were stored at −80° C. until analyzed by ELISA. For post challenge data, samples were collected from survivors 3 weeks after infection.

Clostridium difficile Challenge

C. difficile challenge experiments were performed essentially as described by Warren and colleagues (Chen et al. 2008; Warren et al. 2012). Briefly, mice were pre-adapted to acidic water by adding acetic acid at a concentration of 2.15 μL/mL [v/v] to their drinking water one week prior to antibiotic treatments. Six days prior to infection, an antibiotic cocktail included metronidazole (0.215 mg/mL) (Sigma Aldrich), gentamicin (0.035 mg/mL) (Wisent), vancomycin (0.045 mg/mL) (Sigma Aldrich), kanamycin (0.400 mg/mL) (Wisent), colistin (0.042 mg/mL) (Sigma Aldrich) was added to the drinking water. After 3 days, regular water was returned and 24 hours prior to infection, mice received clindamycin (Sigma Aldrich: 32 mg/kg) intraperitoneally in 0.2 mL of PBS using a 28 G needle. Fresh C. difficile cultures were used in the challenge model so the dose used was estimated on the day of infection and the precise inoculum could only be calculated 24 hours later. This procedure led to the use of different C. difficile doses in the two challenge studies performed (1.7×10⁷ or 1.97×10⁵ cfu/mouse). The challenge dose was delivered by gavage in 0.2 ml of meat broth culture media. Mice were then monitored and scored 1-3 times daily for weight loss, activity, posture, coat quality, diarrhea and eye/nose symptoms (Wartren et al. 2012). Mice with a score of 14/20 or above and/or with ≥20% weight loss were considered at a humane endpoint and were euthanized. Any mouse found dead, was given a score of 20. Survivors were followed and euthanized approximately 3 weeks after infection.

Antibody Quantification

Anti-Toxin Antibodies

Whole toxin A (List Biologicals, Campbell, Calif.) or recombinant rbdB were used to coat U-bottom high-binding 96-well ELISA plates (Greiner Bio-one, Frickenhausen, Germany). A standard curve was included on each plate using mouse IgG antibodies (Sigma Aldrich) or mouse IgA antibodies (Sigma Aldrich). Plates were coated with 50 μL of Toxin A (1.0 μg/mL), rbdB (0.25 μg/mL) or IgG/IgA standards overnight at 4° C. in 100 mM bicarbonate/carbonate buffer (pH 9.5). Wells were washed with PBS 3× then blocked with 150 μL of 2% bovine serum albumin (BSA; Sigma Aldrich) in PBS-Tween 20 (0.05; Fisher Scientific) (blocking buffer) for 1 hour at 37° C. Serum samples were heat-inactivated at 56° C. for 30 minutes before a 1:50 dilution in blocking buffer. Intestinal supernatants were added to the plates neat. All sample dilutions including standard curve dilutions were assayed in duplicate (50 μL/well). Plates were incubated for 1 hour at 37° C. then washed 4× with PBS prior to the addition of either HRP-conjugated anti-mouse total IgG antibodies (Sigma Aldrich: 75 μL/well at 1:20 000 in blocking buffer) or HRP-conjugated anti-mouse IgA antibodies (Sigma Aldrich: 75 mL/well at 1:10 000 in blocking buffer). Plates were incubated for an additional 30 minutes (IgG) or 1 hour (IgA) at 37° C. before the addition of 100 μL/well of 3,3′,5,5′-tetramethyl benzidine (TMB) detection substrate (Millipore, Billerica, Mass.). Reactions were stopped after 15 minutes with 0.5 M H₂SO₄. Plates were read at 450 nm on an EL800 microplate reader (BioTek, Instruments Inc., Winooski, Vt.). The concentration of antigen-specific antibodies in each well (ng/mL) was estimated by extrapolation from the standard curve.

Statistical Analysis

Statistical analysis was performed using GraphPad Prism 6 software. For analysis of antibody titers, one-way non-parametric Kruskal-Wallis ANOVA was performed with Dunn's multiple comparison analysis comparing all groups. Statistical significance was considered to have been achieved when p≤0.05. Data are presented as means±standard deviation (SD). For analysis of survival, the log rank (Mantel-Cox) test was used to compare all groups to the PBS control group. The Bonferroni method was used to correct for multiple comparisons. In Table 4, correlations are based on Spearman's r coefficient (non-parametric), 95% Confidence Intervals were calculated, and two tailed ‘p’ values were determined.

Results

Transformed S. Typhimurium YS1646 Expresses Heterologous Antigen

Plasmids expressing the RBDs of Toxin A (rbdA) or Toxin B (rbdB) under the control of different promoters and secretory signals were constructed (FIG. 1). The promoter-secretory signal combinations included SPI-I- (eg: SopE2, SptP) and SPI-II-specific pairings (eg: SseJ, SspH2) as well as pairings used by both SPI-I and SPI-II secretory pathways (eg: SteA, SteB, SspH1). Some of the secretory signals were also paired with inducible promoters nirB, pagC, and lac (Table 1). All primers used in the study are listed in Table 2. A set of plasmids with the same promoter/secretory signal pairings but expressing enhanced green fluorescent protein (EGFP) were also constructed. All plasmids were transformed into S. Typhimurium YS1646.

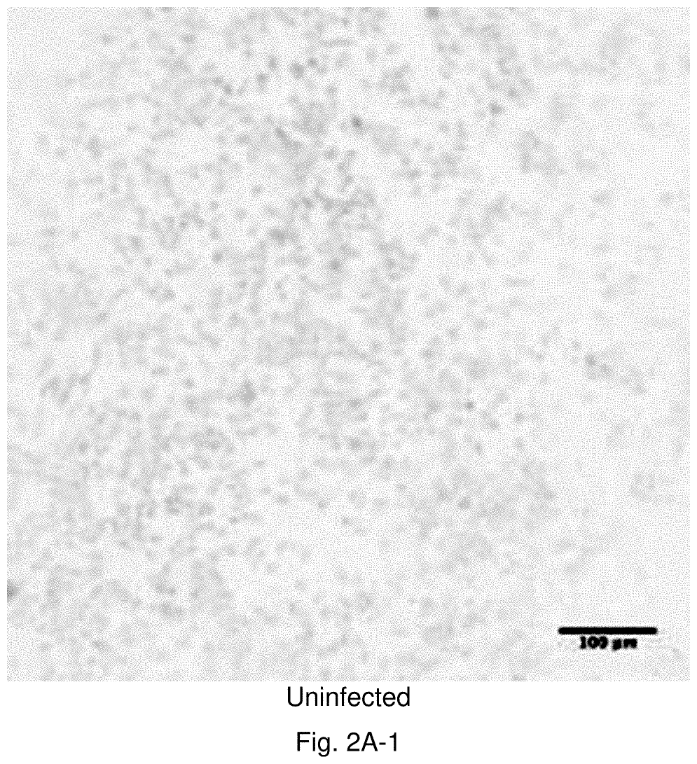

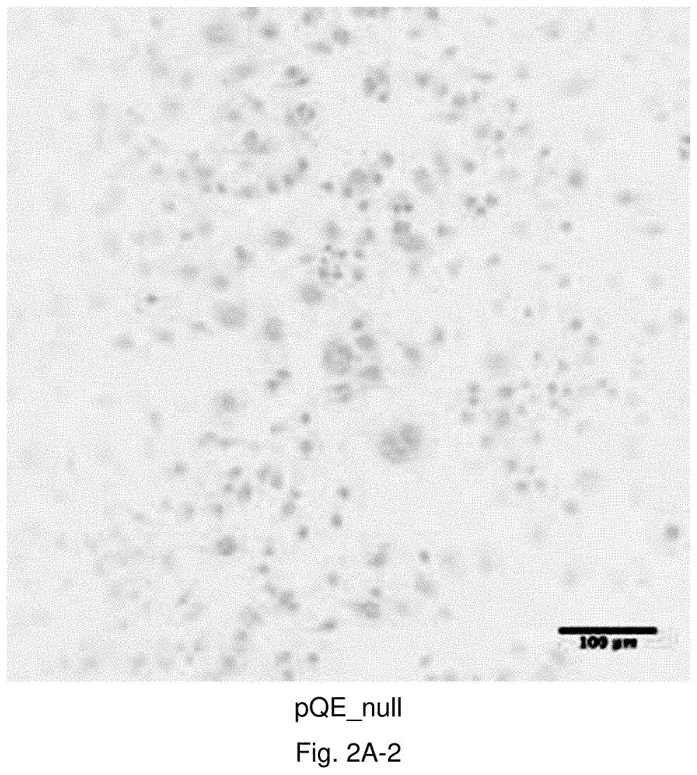

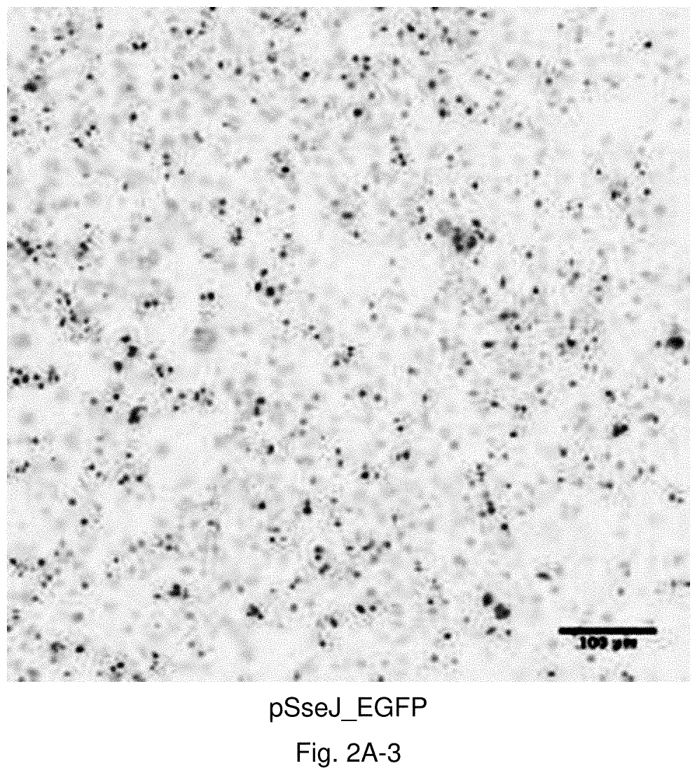

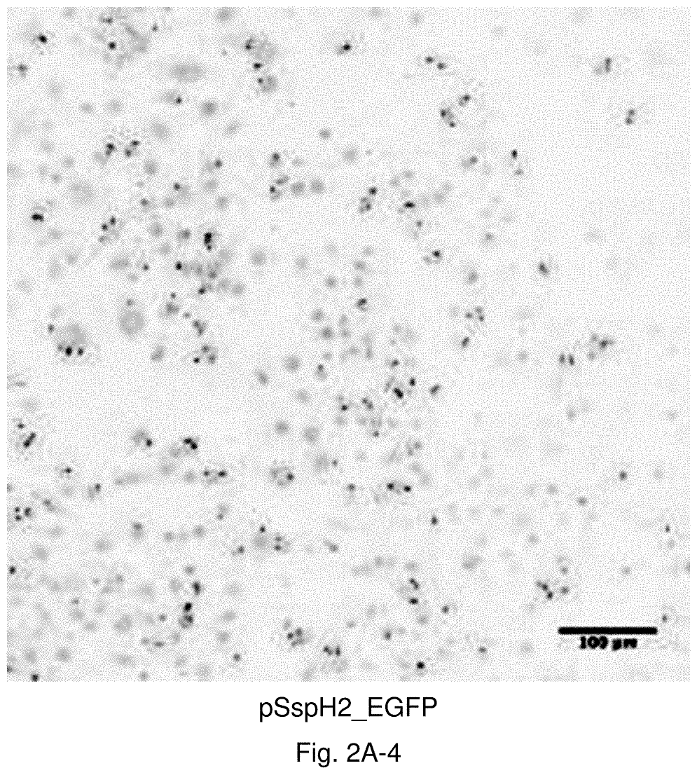

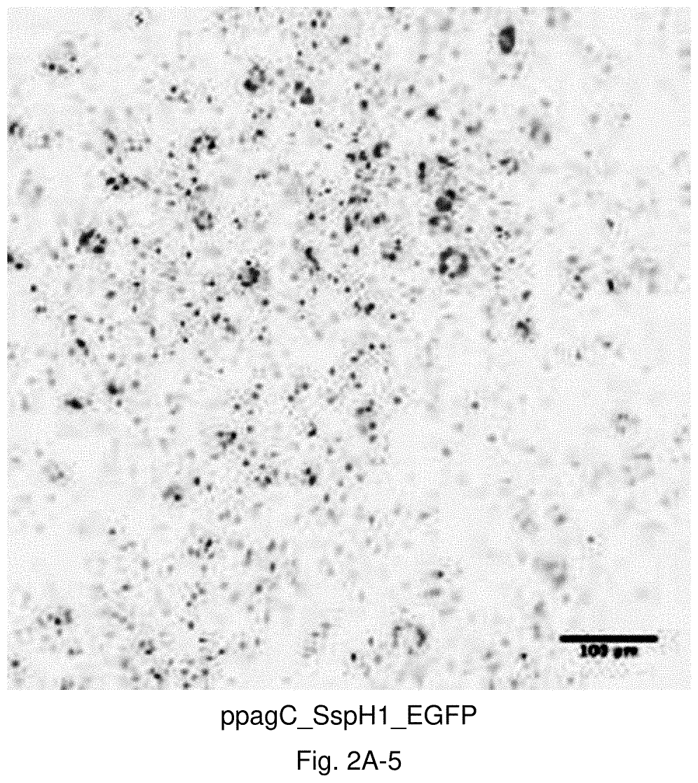

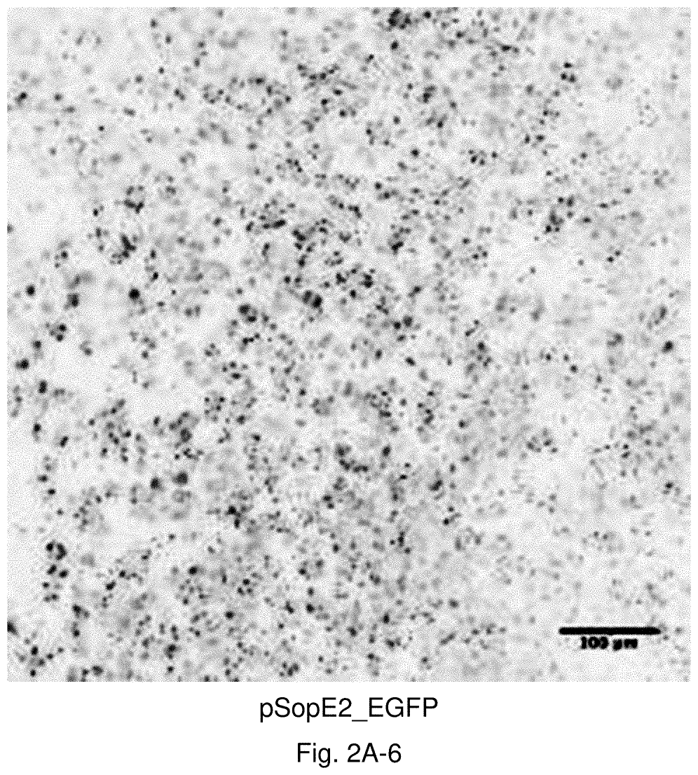

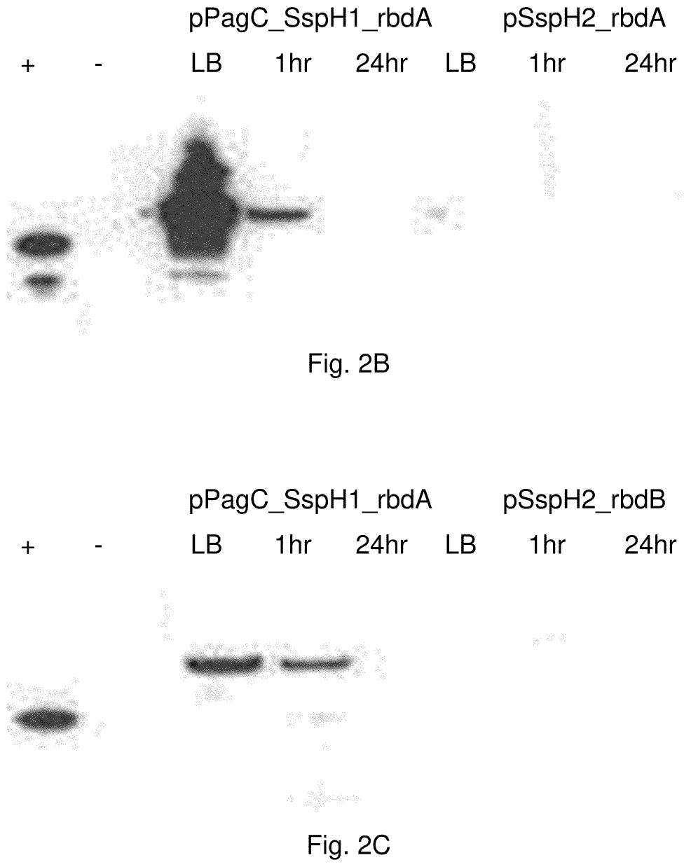

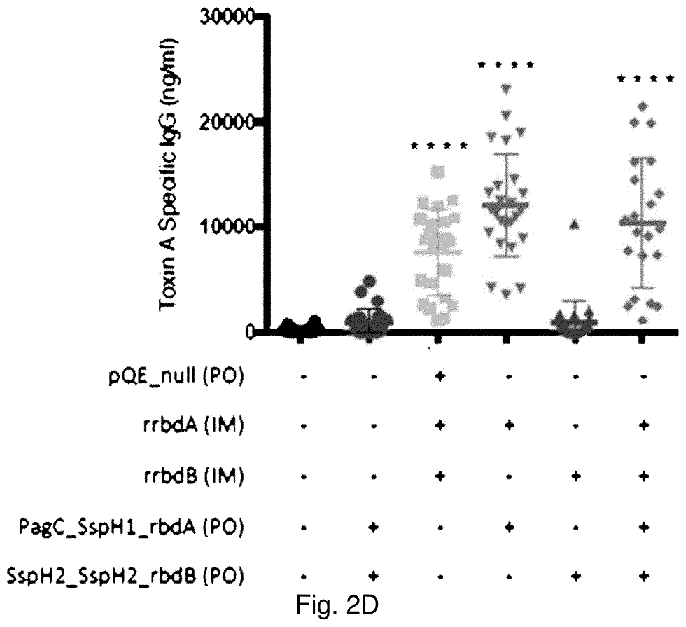

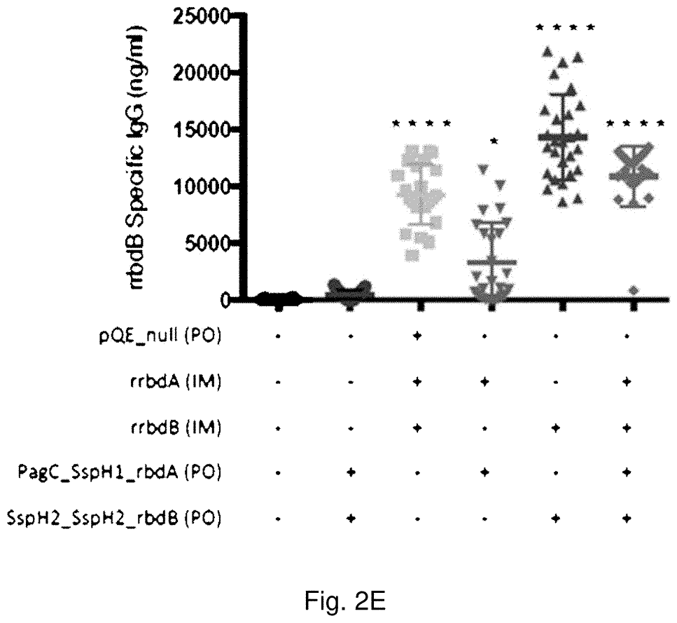

Using the EGFP-expressing strains, antigen expression in monomicrobial culture and during in vitro infection of murine RAW 264.7 macrophages was screened. Most strains produced detectable EGFP in monomicrobial culture (summarized in Table 3). The YS1646 candidates were readily macropinocytosed and a fluorescent signal was detected for all of the EGFP expressing strains (FIG. 2A). Expression varied considerably between strains however with the strongest signal driven by the pagC_SspH1_EGFP construct. Some constructs (eg: SspH2_SspH2_EGFP) had good initial EGFP expression but survival and/or replication in the macrophages was markedly reduced at 24 hours post-infection.

Expression of the targeted C. difficile RBDs in monomicrobial culture and murine macrophages was examined by Western blotting at 1 and 24 hours post-infection. Modest production of rbdA and rbdB could be documented by most strains in monomicrobial culture, but very few strains had detectable antigen expression during macrophage infection (rbdA in FIG. 2B; rbdB in FIG. 2C) (summarized in Table 3). For example, the pagC_SspH1 pairing drove strong expression of both antigens in broth and at 1-hour post-infection in the murine macrophages but the SspH2_SspH2 pairing failed to drive detectable rbdB expression and the level of rbdA production was barely detectable only in monomicrobial culture. Secretion of the RBDs into extracellular medium was examined in monomicrobial culture (Table 3). Only pagC_SspH1_rbdA had detectable antigen secretion. The lack of secretion detection may be due to low levels of expression in the cells.

The most promising constructs were advanced to mouse immunogenicity testing. Since neither monomicrobial culture nor RAW 264.7 cells are adequate models for the low oxygen tension and poly-microbial environment of the gastrointestinal tract, and some of the apparently negative constructs were included in the in vivo immunogenicity testing.

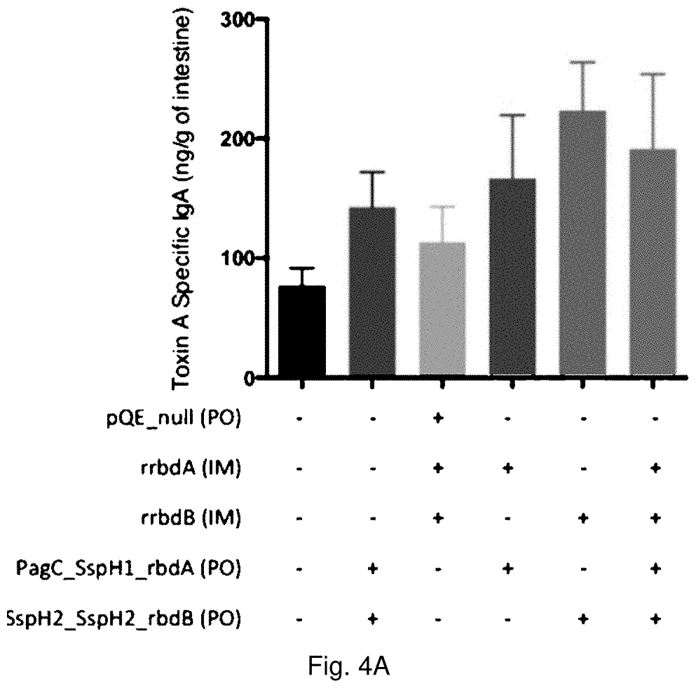

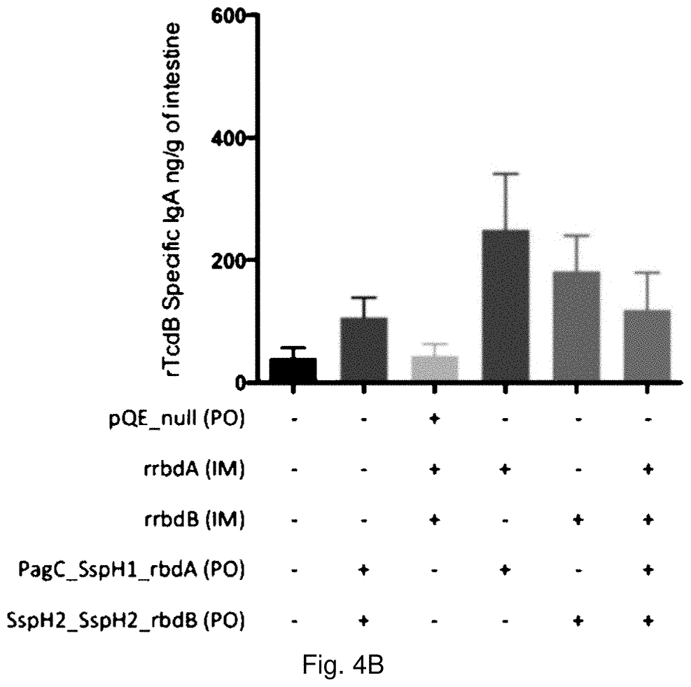

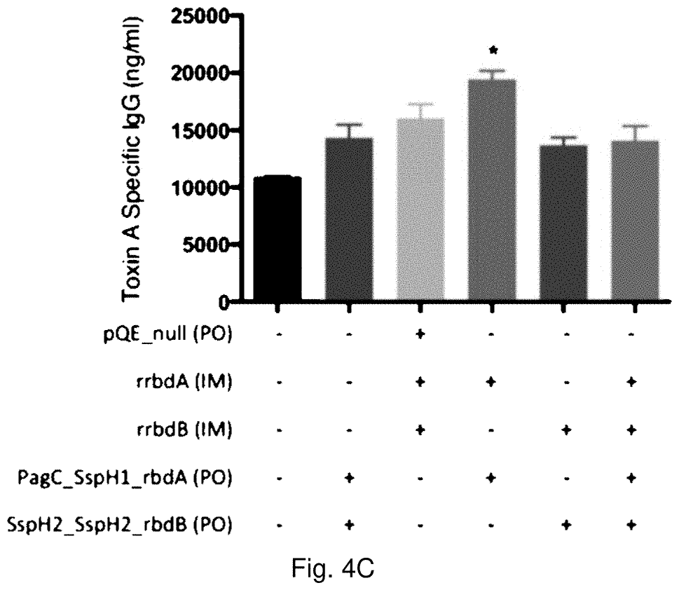

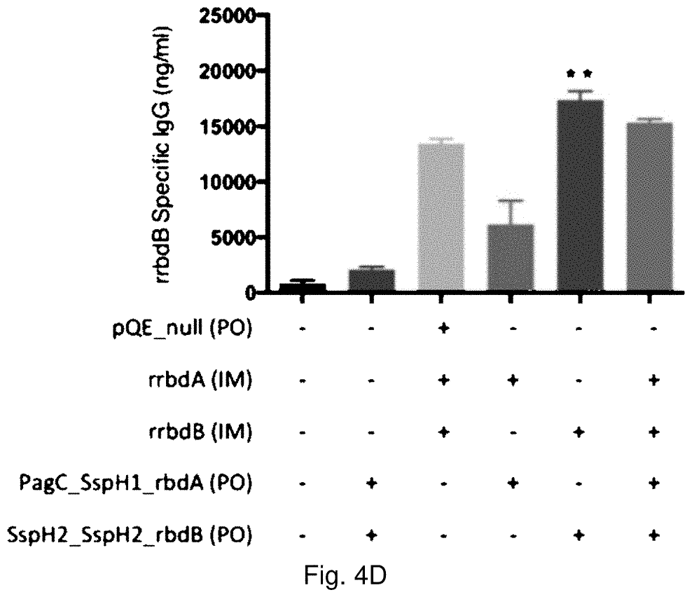

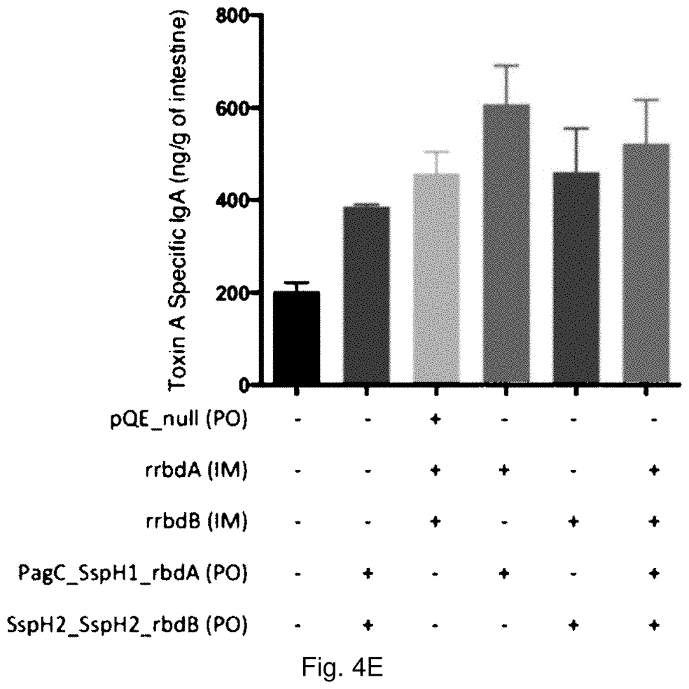

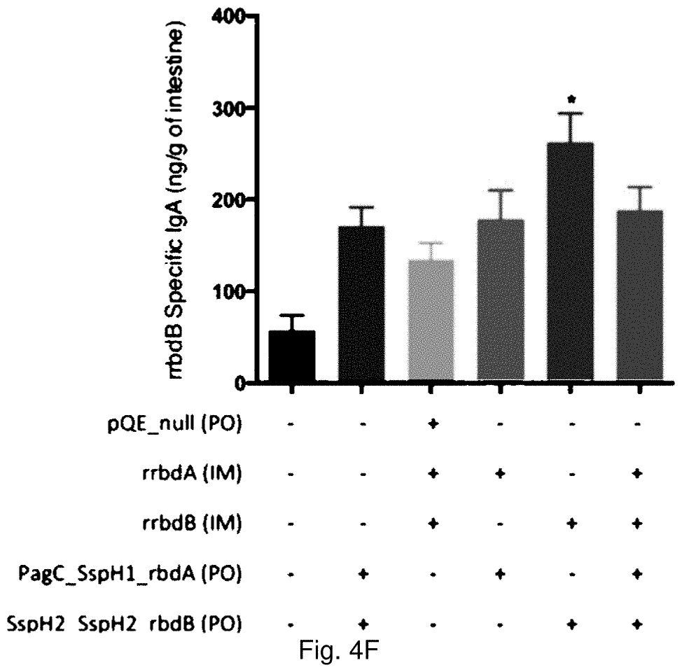

FIGS. 4A-4F show that vaccination with antigen expressing YS1646 increases antibody titers in intestines mice pre-challenge and in post-challenge survivors. Mice were immunised with a dose of 10 pg recombinant antigen (rrbdA and/or rrbdB) intramuscularly, and three doses of 1×10⁹ cfu of antigen expressing YS1646 (pagC_SspH1_rbdA and/or SspH2_SspH2_rbdB), orally every other day. 5 weeks after vaccination, mice were either euthanised and intestines were collected or challenged with 1.7×10⁷ cfu of C. difficile. 3 weeks after infection, serum and intestines were collected from survivors. Pre-challenge intestinal toxin A-specific IgA antibodies (FIG. 4A) and rbdB specific IgA antibodies (FIG. 4B) were detected by ELISA (n=4-5, one experiment). Post-challenge serum toxin A-specific IgG antibodies (FIG. 4C) and rbdB specific IgG antibodies (FIG. 4D) were detected by ELISA. Intestinal toxin A-specific IgA antibodies (FIG. 4E) and rbdB specific IgA antibodies (FIG. 4F) were detected by ELISA (n=2-8, one experiment). Mean and standard error of the mean (SEM) are shown. Kruskal-Wallis test and Dunn's Multiple Comparison test were used to compare between all groups. *p<0.05, **p<0.01, ***p<0.001, ****p<0.0001 compared to the PBS control group.

rbdA and rbdB Delivered by YS1646, in Combination with Recombinant rbdA/rbdB, is Highly Immunogenic in Mice

Using the rapid induction of serum antigen-specific IgG as the principal screening tool, a multimodality schedule was identified as the most promising vaccination strategy. This schedule was comprised of a single IM dose of the recombinant RBD (rrbd) on day 0 with 3 PO doses of the corresponding RBD-expressing strain on days 0, 2 and 4. When sera were collected 3-4 weeks after vaccination using this schedule, rbdA-specific (FIG. 2D) and rbdB-specific (FIG. 2E) IgG titers were consistently elevated. In contrast, mice that received only the three PO doses of YS1646 strains bearing the RBD antigens had no detectable serum IgG response. Despite the failure to induce IgG with PO vaccination, three doses of YS1646 on alternate days could nonetheless prime for a significant response to a subsequent IM booster dose delivered 3 weeks later (data not shown). This multimodality schedule generated IgG responses that were consistently higher than those achieved by recombinant antigen delivered IM and pQE_null strain delivered PO but these differences did not reach statistical significance (p=0.1727 for rbdB). 5 weeks after vaccination, mice given antigen by both IM and PO vaccination tended to have higher mucosal IgA responses in the intestine compared to mice vaccinated intramuscularly with recombinant antigen and pQE_null PO. (FIG. 4A, 4B).

Selection of Candidate YS1646 Strains for Challenge Testing

The combined screening studies identified two YS1646 constructs that were carried forward into challenge testing (pagC_SspH1_rbdA and SspH2_SspH2_rbdB) (Table 3). Since oral immunization generated intestinal IgA (FIG. 4A, 4B) and was able to prime animals for a strong systemic IgG response to a subsequent IM boost (data not shown), PO-only groups were included in challenge studies in addition to the multi-modality IM+PO schedule.

YS1646-Vectored rbdA and rbdB Vaccines Protect Mice from Lethal C. difficile Challenge

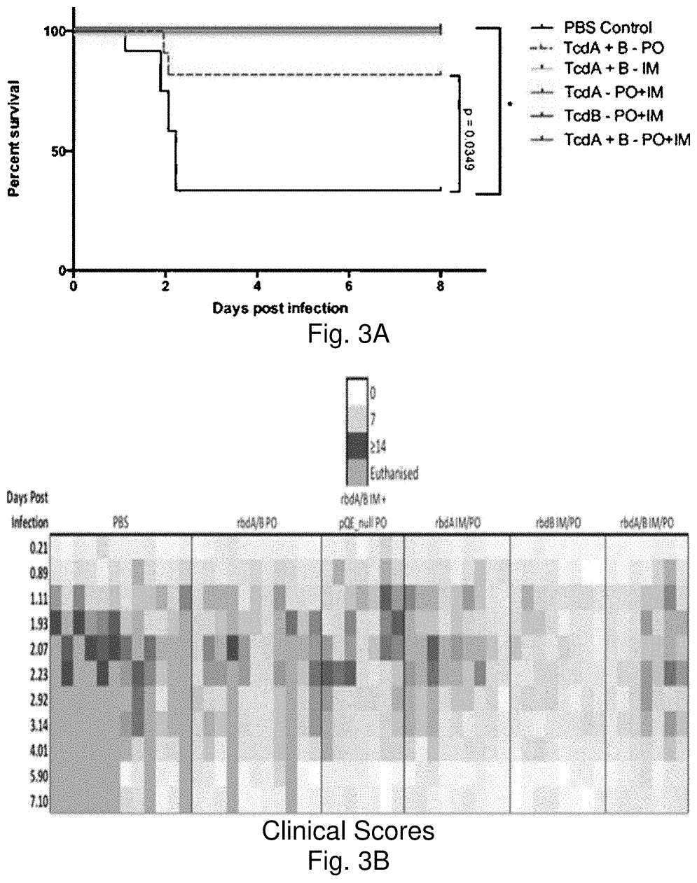

5 weeks after vaccination, mice were challenged with a lethal dose of C. difficile bacteria and monitored for weight loss, clinical score and death. Overall, 67% of the PBS control group succumbed to infection between 36 and 72 hours post-infection (FIG. 3A). Only 18% of mice that received three PO doses of the pagC_SspH1_rbdA and SspH2_SspH2_rbdB strains, succumbed to the infection. All other vaccinated groups had 100% survival (FIG. 3A). The recovery of animals that survived appeared to be complete: surviving mice recovered their original body weight had very low or completely normal clinical scores by 6 days post infection. Mice were followed for up to 3 weeks after infection and no relapses were observed. During infection, mice were ‘clinically’ scored 1-3 times daily (FIG. 3B). Although the groups vaccinated with rrbdA+rrbdB IM and the pQE_null strain PO had 100% survival, 71% of these mice were severely ill: achieving a score of 12 or higher (14=animal care cut-off for humane endpoint). The proportion of severely ill mice in groups that received any antigen-expressing YS1646 strain with an IM dose of recombinant protein was consistently much lower (0%-14%). None of the animals in the group that received rrbdB IM plus three doses of the SspH2_Ssph2_rbdB strain PO experienced severe illness. There is a strong negative correlation between serum anti-rbdB IgG, both before and after challenge, and the highest clinical score achieved by individual mice (FIG. 2E; FIGS. 4B and 4D; Table 4). The results suggest that in the mouse model, an immune response directed towards TcdB is sufficient to obtain effective protection from C. difficile challenge.

FIGS. 2A, 2B, 2C, 2D and 2E show transformed YS1646 strains expressed heterologous antigen. The photomicrographs are color-inverted and contrast optimized for reproduction. FIG. 2A shows EGFP expressing strains of YS1646 were added to RAW 264.7 macrophages in vitro. 24 hours after infection cells were visualized using a fluorescent microscope. Images are representative of two repeats. Receptor binding protein expression was examined by western blot. YS1646 strains transformed with rbdA, shown in FIG. 2B and rbdB, shown in FIG. 2C, plasmids were grown for 16 hours in LB or used to infect RAW 264.7 macrophages for 1 or 24 hr. Gels were run with a positive control (recombinant antigen) and film was exposed for 2 minutes. The recombinant RBDs expressed in E. coli do not contain secretion signals. The increased size of the RBDs produced in YS1646 are consistent with the secretion signal used in the plasmid which is not cleaved. Mice were immunised with a dose of 10 μg recombinant antigen (rrbdA and/or rrbdB) intramuscularly, and three doses of 1×10⁹ cfu of antigen expressing YS1646 (pagC_SspH1_rbdA and/or SspH2_SspH2_rbdB), orally every other day. Serum was collected 3-4 weeks after vaccination Toxin A specific IgG, shown in FIG. 2D or rbdB specific IgG, shown in FIG. 2E was detected by ELISA (n=21-28, 4 repeats). Mean and standard deviation (SD) are shown. Kruskal-Wallis test and Dunn's Multiple Comparison test were used to compare between all groups. *p<0.05, **p<0.01, ***p<0.001, ****p<0.0001 compared to the PBS control group.

FIGS. 3A and 3B show vaccination with antigen expressing YS1646 strains protected against C. difficile challenge. Mice were immunised with a multimodality schedule, 10 ug of recombinant antigen intramuscularly, and three doses of 1×10⁹ cfu of antigen expressing YS1646, orally every other day. Mice were given either PBS orally and intramuscularly (PBS), pagC_SspH1_rbdA and SspH2_SspH2_rbdB orally (rbdA/B PO), both rrbdA and rrbdB intramuscularly with pQE_null orally (rbdA/B IM+pQE_null PO), rrbdA intramuscularly and pagC_SspH1_rbdA orally (rbdA IM/PO), rrbdB intramuscularly and SspH2_SspH2_rbdB orally (rbdB IM/PO), or both rrbdA and rbdB intramuscularly and pagC_SspH1_rbdA and SspH2_SspH2_rbdB orally (rbdA/B IM/PO). 5 weeks after vaccination, mice were challenged with 1.7×10⁷ cfu and 1.97×10⁵ cfu of C. difficile. Mice were clinically scored 1-3 times daily. A score of 314/20 and/or >20% loss of the starting body weight were considered humane endpoints. Survival (FIG. 3A) and clinical scores (FIG. 3B) are shown (n=7-12, 2 repeats). Log rank (Mantel-Cox) test was used to compare all groups to the PBS control group. Correction of the p value for multiple comparisons was done using the Bonferroni method. *p<0.01 compared to the PBS control group.

The combined IM+PO schedules also elicited small but detectable increases in antigen-specific IgA levels in the intestinal tissues after challenge although the increase only reached statistical significance for the animals vaccinated against rbdB alone (p<0.05 versus the control group) (FIG. 4E, 4F). Interestingly, the intestinal anti-rbdB IgA levels tended to be slightly lower in the animals that received both of the YS1646 constructs PO compared to those vaccinated only against rbdB (FIG. 4B, 4F) although this difference also failed to reach statistical significance.

Discussion

The pathology associated with CDI is thought to be toxin-mediated (Ananthakrishnan et al. 2010) and there are strong precedents for the efficacy of vaccine-induced anti-toxin antibodies in the prevention or modification of toxin-mediated diseases (eg: tetanus, diphtheria, cholera) (Donald et al. 2013; Tian et al, 2012). Indeed, an anti-TcdB monoclonal antibody (bezlotoxumab or Zinplava™: Merck) has recently been shown to reduce the frequency of recurrent C. difficile disease (Wilcox et al. 2017). In addition to passive immunotherapy, the generation of anti-toxin antibodies is also the predominant strategy being pursued by both large and small pharmaceutical companies with an interest in developing C. difficile vaccines (Bruxelle et al. 2018). However, the most advanced of these candidate vaccines require multiple doses of antigen with an adjuvant over several months to achieve high serum antibody concentrations (Bruxelle et al. 2010; Kociolek et al. 2016). Furthermore, even though CDI is a disease of the gastrointestinal mucosa, none of these candidates would be expected to generate an effective mucosal immune response. In theory at least, the delivery of the same C. difficile toxin antigens using a live-attenuated S. Typhimurium vector should be able to induce both local and systemic immunity. There are several groups working on delivering C. difficile antigen at the mucosal surface (Hong et al, Wang et al). Recently, Wang et al used a non-toxigenic C. difficile to target TcdB and TcdA (Wang et al). They found that after 3 doses delivered every two weeks, that their vaccine candidate was effective at protecting mice and hamsters. A multi-modality vaccination schedule using a single IM dose of recombinant toxin A and/or toxin B receptor binding domain proteins with PO delivery of YS1646 bearing the same RBD antigens over a five-day period was demonstrated to rapidly induce both systemic and mucosal responses and protect mice from an otherwise lethal challenge. The induction of an effective local immune response by the YS1646-vectored vaccines was strongly supported by the fact that oral vaccination alone could also provide substantial protection in the absence of detectable serum antibodies prior to challenge.

Although logistically more complicated and considered ‘inelegant’ by some, heterologous prime-boost and multi-modality vaccination strategies are gaining traction for a wide range of infections and other complex conditions, such as cancers (Kardani et al. 2016; Lakhashe et al. 2014; Luke et al. 2014). Of particular interest to the current proposal, such combined modality approaches have shown promise in eliciting effective immune responses against mucosal pathogens such as HIV/SHIV and influenza (Lakhashe et al. 2014; Luke et al. 2014). Combined modality strategies may also have a place in toxin-mediated diseases in which high titres of preformed antibodies are needed such as Clostridium perfringens infection (Solanki et al. 2017) or when a rapid but sustained response is desirable such as Ebola (Shukarev et al. 2017). These new approaches have the potential to enhance the character, kinetics and durability of the response (Knudsen et al, 2014). The multi-modality method developed in the murine model would be relatively easy to administer to the ‘typical’ person who might benefit from a C. difficile vaccine: i.e., those in or entering a long-term care facility (van Kleef et al. 2016) or being prepared for elective surgery. Only one face-to-face clinic/office visit would be needed to receive the IM vaccine and the first (supervised) PO vaccine after which the remaining two PO doses on alternate days could be taken autonomously (as is currently the practise for the live-attenuated S. typhi Ty21a vaccine). The long clinical experience with Ty21a also confirms the feasibility of delivering an attenuated Salmonella to the intestinal tissues (Galen et al. 2016). Although such a rapid vaccination schedule would likely increase compliance, it is also possible that the durability of the response would be compromised (Prisco et al. 2012).

Even though wild-type S. Typhimurium typically causes only mild disease localized to the gastrointestinal tract in humans (Galen et al. 2016), it can sometimes cause invasive disease with serious outcomes (Haselbeck et al. 2017). The YS1646 strain that is the backbone of the preferred vaccine platform carries mutations of both an LPS gene (msbB) and a part of the purine production machinery (purI) that render it highly attenuated (Toso et al. 2002). Although the mechanisms of attenuation differ, the live-attenuated Ty21a S. typhi vaccine has an excellent safety record, even in elderly subjects (Galen et al. 2016). In the critical development pathway of YS1646 as a possible anti-cancer agent in the early 2000s, this strain proved to be safe in multiple small (eg: mice, rats) and large animal models (e.g., dogs, Rhesus macaques) (D. Bermudes, unpublished data) before it was permitted to advance to a phase 1 clinical trial (Toso et al 2002). In this trial, a single dose of up to 3×10⁸ colony-forming units (cfu) of YS1646 was administered intravenously to 24 subjects with metastatic melanoma or renal cell carcinoma without any major safety signals. Most of the subjects in this trial cleared YS1646 from their blood stream in <12 hours (Toso et al. 2002). It was subsequently suggested that an unexpected susceptibility of YS1646 to physiologic levels of CO₂ present in human tissues (˜5%) may have contributed to its failure as a cancer therapy (Karsten et al. 2009). In contrast to the need for YS1646 to disseminate and replicate actively in tumour tissues as an anti-cancer agent, to be an effective vaccine vector, YS1646 only needs to invade locally and express the targeted antigen for a short period of time in the GALT (Galen et al. 2016). A preferred YS1646 as a C. difficile vaccine provides chromosomal integration of the TcdA and TcdB RBD constructs. Chromosomal integration will typically reduce the copy number of the target gene and therefore protein expression, these effects will be mitigated through the use of strong promoters (eg: PpagC) and the integration of tandem repeats for both antigens. Since several current prototypes were able to elicit immune responses despite undetectable antigen production in vitro, chromosomally integrated strains may be designed that are immunogenic. Other clinical experience with attenuated S. Typhimurium includes Hindle et al. 2002, who exposed a small number of human subjects to a single oral dose of up to 1×10⁹ cfu of a strain bearing aroC and SPI-II T3SS mutations without dissemination or ill effects, and observed asymptomatic shedding of an attenuated S. Typhimurium strain for 3 weeks in the feces of patients, with all shedding ending by week 4 after vaccination. Although YS1646 has different attenuating mutations and may have a different colonization profile in humans after oral delivery, asymptomatic persistence of this S. Typhimurium strain was also demonstrated for at least 1 week in a small proportion of subjects after intravenous delivery in the early anti-cancer phase 1 trial (Toso et al. 2002). The mere fact of persistence of YS1646-vectored C. difficile vaccine does not automatically disqualify a vaccine candidate. Indeed, several of the live-attenuated vaccines on the market are routinely shed by vaccinees for longer than a week. These include rotavirus that is shed for up to 9 days post vaccination (Yen et al. 2011), measles that can be detected for at least 14 days (Rota et al. 1995), oral polio that can persist for several months (Troy et al. 2014) and varicella that causes a lifelong latent infection (Freer et al. 2018).

First, there is no perfect small or large animal model for human CDI (Best et al. 2012; Cohen et al. 2014). Although mice are widely considered to be one of the most informative models, mice are also the natural host for S. Typhimurium. Indeed, S. Typhimurium infection in mice is commonly used as a model for human typhoid fever caused by S. Typhi (Santos et al. 2001). Although mice remained completely healthy during and after oral vaccination, colonization of the spleen and liver was observed by some of the YS1646 strains carrying either TcdA or TcdB constructs for 1-2 weeks after vaccination (data not shown). Although such dissemination is not expected in humans due to the CO₂ sensitivity of YS1646, it is certainly possible that immunity generated in response to persistent antigen expression over days-weeks will differ from that induced by a shorter exposure. A mutation in zwf may be used to decrease CO₂, pH, and osmolarity sensitivity. The murine model is believed to be predictive of outcomes in humans with the known limitations.

The choice of optimal promoter-secretory signal pairings for in vivo expression of the RBD antigens is complicated a difficulty in testing the conditions to which the YS1646 strains will be exposed in the human gastrointestinal tract and the GALT. This risk is mitigated by using a multi-layered screening process, but some already identified constructs that do not appear to produce the targeted RBD in vitro (in monomicrobial culture or RAW 264.7 cells), but still elicit strong antibody responses in the mouse model.

A live-attenuated S. Typhimurium strain (YS1646) was repurposed as a vaccine-vector to target the major toxins of C. difficile. Administered in a 5-day, multi-modality schedule (IM×1 plus PO×3), these candidate vaccines elicited high serum IgG titres and provided complete protection from lethal challenge in a mouse model.

Each reference cited herein is expressly incorporated herein in its entirety. Such references provide examples representing aspects of the invention, uses of the invention, disclosure of the context of the invention and its use and application. The various aspects disclosed herein, including subject matter incorporated herein by reference, may be employed, in combination or subcombination and in various permutations, consistent with the claims.

The invention may be embodied in other specific forms without departing from its spirit or essential characteristics. The described embodiment is to be considered in all respects only illustrative and not restrictive. The scope of the invention is, therefore, indicated by the appended claims rather by the foregoing description. All changes that fall within the meaning and range of equivalency of the claims are to be embraced within their scope.

TABLE 1

| | | Secretory | |
|---|---|---|---|
| Plasmid | Promoter | Signal | Antigen |
| pQE_null | — | — | — |
| pSopE2_SopE2_rbdB | SopE2 | SopE2 | TcdB1821-2366 |
| pSseJ_SseJ_rbdB | SseJ | SseJ | TcdB1821-2366 |
| pSptP_SptP_rbdB | SptP | SptP | TcdB1821-2366 |
| pSspH1_SspH1_rbdB | SspH1 | SspH1 | TcdB1821-2366 |
| pSspH2_SspH2_rbdB | SspH2 | SspH2 | TcdB1821-2366 |
| pSteA_SteA_rbdB | SteA | SteA | TcdB1821-2366 |
| pSteB_SteB_rbdB | SteB | SteB | TcdB1821-2366 |
| ppagC_SspH1_rbdB | pagC | SspH1 | TcdB1821-2366 |
| pSspH2_SspH2_rbd | SspH2 | SspH2 | TcdA1820- |
| plac_SopE2_rbdA | lac | SopE2 | TcdA1820- |
| plac_SspH1_rbdA | lac | SspH1 | TcdA1820- |
| pnirB_SopE2_rbdA | nirB | SopE2 | TcdA1820- |
| pnirB_SspH1_rbdA | nirB | SspH1 | TcdA1820- |
| ppagC_SopE2_rbdA | pagC | SopE2 | TcdA1820- |
| ppagC_SspH1_rbdA | pagC | SspH1 | TcdA1820- |

TABLE 2
Primers used in Plasmid Construction

| | Forward Primer | Reverse Primer | |
|---|---|---|---|
| | (5′→3′) | (5′→3′) | Source |
| SopE2 | CCGCTCGAGTAAAA | CATGGTAGTTCTCC | YS1646 |
| promoter and | ATGTTCCTCG | TTTTAG | |
| secretory signal | ATAAA | SEQ ID NO: 002 | |
| | SEQ ID NO: 001 | | |
| SptP promoter | CGCCTCGAGTTTACG | CATTTTTCTCTCCT | YS1646 |
| and secretory | CTGACTCAT TGG | CATACTTTA | |
| signal | SEQ ID NO: 003 | SEQ ID NO: 004 | |
| SseJ promoter | CGCCTCGAGACATA | CGCCTCGAGACAT | YS1646 |
| and secretory | AAACACTAGC ACT | AAAACACTAG | |
| signal | SEQ ID NO: 005 | CACT | |
| | | SEQ ID NO: 006 | |
| SspH1 | CGCCTCGAGCGCTAT | CTCTGCGGCCGCG | YS1646 |
| promoter and | ATCACCAAA AC | GTAAGACCTG | |
| secretory signal | SEQ ID NO: 007 | ACGCTC | |
| | | SEQ ID NO: 008 | |
| SspH2 | CGCCTCGAGGTTTGT | CTCTGCGGCCGCA | YS1646 |
| promoter and | GCGTCGTAT | TTCAGGCAGG | |
| secretory signal | SEQ ID NO: 009 | CACGCA | |
| | | SEQ ID NO: 010 | |
| SteA promoter | CGCCTCGAGGTTT | CTCTGCGGCCGCA | YS1646 |
| and secretory | CGCCGCATGTT G | TAATTGTCCA | |
| signal | SEQ ID NO: 011 | AATAGT | |
| | | SEQ ID NO: 012 | |
| SteB promoter | CGCCTCGAGCGCTC | CTCTGCGGCCGCT | YS1646 |
| and secretory | CAGCGCTTCG A | CTGACATTAC | |
| signal | SEQ ID NO: 013 | CATTT | |
| | | SEQ ID NO: 014 | |
| Lac promoter | CGCCTCGAGCATTA | | Sequence is in the |
| | GGCACCCCAG | | primers |
| | GCTTTACACTTTAT | | |
| | GCTTCCGGCTC | | |
| | GTATGTTGTGTGGA | | |
| | ATTGTGAGCG | | |
| | GATAA, | | |
| | SEQ ID NO: 015 | | |
| | GTGGAATTGTGAGCG | | |
| | GATAACAAT | | |
| | TTCACACAGGAAACA | | |
| | GCTATGACC | | |
| | ATGACTAACATAACA | | |
| | CTATCCAC | | |
| | SEQ ID NO: 016 | | |
| nirB promoter | CGCCTCGAGTTGTGG | CGCGCGGCCGCCG | DH5D E. coli |
| | TTACCGGCC CGAT | GATCTTTACT | |
| | SEQ ID NO: 017 | CGCATTAC | |
| | | SEQ ID NO: 018 | |
| pagC promoter | CGCCTCGAGGTTAAC | AACAACTCCT | YS1646 |
| | CACTCTTAA TAA | TAATACTACT | |
| | SEQ ID NO: 019 | SEQ ID NO: 020 | |
| SopE2 | GGCGGTAATAGAAA | AAGTCGCGGCCGC | YS1646 |
| Secretion | AGAAATCGA | CGGATCTTTA | |
| Signal | GGCAAAAATGACTA | CTCGC | |
| | ACATAACACT | SEQ ID NO: 022 | |
| | ATCCAC | | |
| | SEQ ID NO: 021 | | |
| SspH1 | GGCGGTAATAGAAA | CTCTGCGGCCGCG | YS1646 |
| Secretion | AGAAATCGA | GTAAGACCTG | |
| Signal | GGCAAAAATGTTTA | ACGCTC | |
| | ATATCCGCAA | SEQ ID NO: 024 | |
| | TACACAACCTT | | |
| | SEQ ID NO: 023 | | |
| rTcdA | CGCGCGGCCGCGAC | TAGTCGGCGCGCC | VPI 10463 |
| | TTATTACTAT GAT | CGCCATATAT | |
| | SEQ ID NO: 025 | CCCAGG | |
| | | SEQ ID NO: 026 | |
| rTcdB | CCGGCGGCCGCAGA | AGTCGGCGCGCCG | VPI 10463 |
| | GAAATTTTAT | TTCACTAATC | |
| | ATTAAT | ACTAATTG | |
| | SEQ ID NO: 027 | SEQ ID NO: 028 | |
| EGFP | CGCGCGGCCGCGGT | AGTCGGCGCGCCT | pEGFP_C1 |
| | GAGCAAGGG CGAG | TACTTGTACA | |
| | SEQ ID NO: 029 | GCTCGTC | |
| | | SEQ ID NO: 030 | |

This table lists the primers used to replicate the sequences from source DNA. Some sequences were further edited to include an ATG start site between the promoter and secretory signal.

TABLE 3
In vitro and in vivo screening of plasmids

| | In vitro | | | | | | | | | |
|---|---|---|---|---|---|---|---|---|---|---|
| | EGFP Detection | | | | | | | | | |
| | (EGFP expressing | | | | | | | | | |
| | strains) | | Antigen Detection by WB | | | | | | | |
| | | RAW | | | RAW | RAW | In vivo (IM Prime, PO | | | |
| | | 264.7 | | Secretion | 264.7 | 264.7 | Serum IgG | | Intestinal IgA | |
| Strains | [symbol] LB | (24 h) | [symbol] LB | in LB | (1 hr) | (24 h) | rbdB | rbdA | rbdB | rbdA |
| pQE_null | 0 | 0 | 0 | 0 | 0 | 0 | 0 | 0 | 0 | 0 |
| SopE2_SopE2_r [symbol] | +++ | +++ | + | 0 | 0 | 0 | 0 | <ctl | 0 | <ctl |
| SseJ_SseJ_rbdB [symbol] | + | ++ | + | 0 | 0 | 0 | ++ | <ctl | 0 | 0 |
| SptP_SptP_rbdB [symbol] | + | + | + | 0 | + | 0 | + | <ctl | + | 0 |
| SspH1_SspH1_r [symbol] | + | + | 0 | 0 | 0 | 0 | ++ | <ctl | ++ | 0 |
| SspH2_SspH2_r [symbol] | ++ | ++ | 0 | 0 | 0 | 0 | +++ | <ctl | +++ | 0 |
| SteA_SteA_rbd [symbol] | +++ | ++ | + | 0 | 0 | 0 | ++ | <ctl | +++ | 0 |
| SteB_SteB_rbdB [symbol] | + | +++ | 0 | 0 | 0 | 0 | <ctl | <ctl | 0 | 0 |
| pagC_SspH1_rb [symbol] | n/a | +++ | + | n/a | + | 0 | n/a | n/a | n/a | n/a |
| SspH2_SspH2_r [symbol] | ++ | ++ | 0 | n/a | 0 | 0 | n/a | n/a | n/a | n/a |
| lac_SopE2_rbdA [symbol] | + | + | + | 0 | 0 | 0 | <ctl | <ctl | 0 | 0 |
| lac_SspH1_rbdA [symbol] | 0 | 0 | 0 | 0 | 0 | 0 | <ctl | <ctl | 0 | 0 |
| nirB_SopE2_rbd [symbol] | ++ | + | + | 0 | + | 0 | <ctl | <ctl | 0 | ++ |
| nirB_SspH1_rbd [symbol] | ++ | +++ | + | 0 | + | 0 | <ctl | <ctl | 0 | 0 |
| pagC_SopE2_rb [symbol] | n/a | ++ | 0 | 0 | 0 | 0 | <ctl | <ctl | 0 | +++ |
| pagC_SspH1_rb [symbol] | n/a | +++ | + | + | + | 0 | <ctl | <ctl | 0 | +++ |

[symbol]

indicates data missing or illegible when filed

EGFP detection is based on the EGFP expressing strains with the same promoter and secretory signal as the listed strain. Strains that were not assessed are indicated in the table as “n/a”. Detection by Western blot is designated as either antigen is detected “+” or not “0”. For in vivo screening, mice were vaccinated with 10 μg of protein IM (rbdA/rbdB) adjuvanted with alum and three weeks later the response was boosted by the YS1646 strains given by PO in 3 doses (n=2-4 mice/group). Serum and intestines were collected 3 weeks after the boost. Titers are shown compared to the control group of the listed protein delivered IM, boosted with pQE_null strain of YS1646. Titers lower than the control are listed as “<ctl”. Titers that match the control are listed as “0”. Titers higher than the control were divided into three categories; “+”, “++”, “+++” with increasing mean titers.

TABLE 4
Correlations between antibody titers and clinical scores

| | | Tox A | Tox A | Tox A | | | | Salmonella | Salmonella |
|---|---|---|---|---|---|---|---|---|---|
| | | IgG | IgG | IgA | rbdB IgG | rbdB | rbdB | IgG | IgG Post |
| | | [symbol] | [symbol] | [symbol] | [symbol] | [symbol] | [symbol] | [symbol] | [symbol] |
| Mean | p | ns | ns | ns | **** | **** | ** | * | ns |
| Score | value | | | | | | | | |
| (all) | r | / | / | / | −0.735 | −0.6555 | −0.5047 | −0.4031 | / |
| | 95% | / | / | / | (−0.8554, | (−0.8189, | (−0.7278, | (−0.6433, | / |
| | CI | | | | −0.5392) | −0.3939) | −0.1849) | −0.09067) | |
| Mean | p | ns | ns | ns | ** | ** | ** | / | / |
| Score | value | | | | | | | | |
| (vax) | r | / | / | / | −0.7191 | −0.6744 | −0.6708 | / | / |
| | 95% | / | / | / | (−0.9031, | (−0.8857, | (−0.8843, | / | / |
| | CI | | | | −0.3124) | −0.2318) | −0.2257) | | |
| Highest | p | ns | ns | ns | **** | *** | ** | * | ns |
| Score | value | | | | | | | | |
| (all) | r | / | / | / | −0.7177 | −0.6068 | −0.5158 | −0.4031 | / |
| | 95% | / | / | / | (−0.8453, | (−0.7904, | (−0.7348, | (−0.6433, | / |
| | CI | | | | −0.5128) | −0.3234) | −0.1994) | −0.09067) | |
| Highest | p | ns | * | * | ns | ns | * | * | / |
| Score | value | | | | | | | | |
| (vax) | r | / | 0.5643 | 0.6453 | / | / | −0.6238 | −0.3741 | / |
| | 95% | / | (0.0008807, | (0.1283, | / | / | (−0.8653, | (−0.6288, | / |
| | CI | | 0.8558) | 0.8865) | | | −0.1475) | −0.05671) | |

Correlations are based on Spearman's r coefficient (non-parametric), 95% Confidence Intervals were calculated, and a two tailed ‘p’ value were determined.
* p < 0.05,
** p < 0.01,
*** p < 0.001,
**** p < 0.0001 [symbol]
indicates data missing or illegible when filed

What is claimed is:

1. A pharmaceutically acceptable orally-administrable vaccine formulation, comprising:
an attenuated recombinant Salmonella bacterium adapted for colonization of a human gut, expressing at least one antigen corresponding to at least one of C. difficile TcdA and TcdB receptor binding domains; and
a pharmaceutically acceptable carrier adapted to preserve the attenuated Salmonella bacterium through the gastrointestinal tract for delivery in the human gut.

2. The pharmaceutically acceptable orally-administrable vaccine formulation according to claim 1, wherein the at least one antigen is secreted from the Salmonella bacteria by a Salmonella Type 3 secretion system.

3. The pharmaceutically acceptable orally-administrable vaccine formulation according to claim 1, wherein the at least one antigen is selected from the group consisting of at least one of TcdA₅₄₅₈₋₈₁₃₀ and TcdB₅₄₆₁₋₇₀₈₀.

4. The pharmaceutically acceptable orally-administrable vaccine formulation according to claim 1, wherein the at least one antigen is expressed in a fusion peptide with a secretory signal selected from the group consisting of one or more of SopE2, SseJ, SptP, SspH1, SspH2, SteA, and SteB.

5. The pharmaceutically acceptable orally-administrable vaccine formulation according to claim 1, wherein the transcription of the at least one antigen is under control of at least one promoter selected from the group consisting of one or more of SopE2, SseJ, SptP, SspH1, SspH2, SteA, SteB, pagC, lac, nirB, and pagC.

6. The pharmaceutically acceptable orally-administrable vaccine formulation according to claim 1, wherein the at least one antigen is produced based on a chromosomally integrated genetically engineered construct.

7. The pharmaceutically acceptable orally-administrable vaccine formulation according to claim 1, wherein the at least one antigen is produced based on a plasmid genetically engineered construct.

8. The pharmaceutically acceptable orally-administrable vaccine formulation according to claim 1, wherein the at least one antigen is produced based on a chromosomally-integrated genetically engineered construct.

9. The pharmaceutically acceptable orally-administrable vaccine formulation according to claim 8, wherein the chromosomally-integrated genetically engineered construct is genetically stabilized by delection of at least one IS200 element.

10. The pharmaceutically acceptable orally-administrable vaccine formulation according to claim 1, wherein the at least one antigen is produced based on a genetically engineered construct comprising a promoter portion, a secretion signal portion, and an antigen portion.

11. The pharmaceutically acceptable orally-administrable vaccine formulation according to claim 10, wherein the promoter portion and the secretion signal portion are separated by a first restriction endonuclease cleavage site.

12. The pharmaceutically acceptable orally-administrable vaccine formulation according to claim 10, wherein the secretion signal portion and the antigen portion are separated by a second restriction endonuclease cleavage site.

13. The pharmaceutically acceptable orally-administrable vaccine formulation according to claim 10, wherein the genetically engineered construct comprises plasmid, further comprising an antibiotic resistance gene.

14. A recombinant attenuated Salmonella bacterium adapted for growth in a mammal, expressing at least one antigen corresponding to at least one of C. difficile TcdA and TcdB receptor binding domains, adapted to induce an vaccine response to C. difficile after oral administration to the mammal.

15. A method of immunizing a human against infection by C. difficile, comprising orally administering a pharmaceutically acceptable formulation, comprising:
an attenuated recombinant Salmonella bacterium, expressing at least one antigen corresponding to at least one of C. difficile TcdA and TcdB receptor binding domains; and
a pharmaceutically acceptable carrier adapted to preserve the attenuated Salmonella bacterium through the gastrointestinal tract for delivery in the human gut.

16. The method according to claim 15, further comprising administering a second pharmaceutically acceptable formulation comprising at least one purified antigen corresponding to at least one of C. difficile TcdA and TcdB receptor binding domains through a non-oral route of administration.

17. The method according to claim 16, wherein the non-oral route of administration comprises an intramuscular route of administration.

18. The method according to claim 16, wherein the second pharmaceutically acceptable formulation comprises an adjuvant.

19. The method according to claim 16, wherein said administering of the pharmaceutically acceptable formulation and second pharmaceutically acceptable formulation are concurrent.

20. The method according to claim 16, wherein said administering of the pharmaceutically acceptable formulation is preceded by the administering of the second pharmaceutically acceptable formulation, according to a prime-pull administration protocol.
